(19) United States
(12) Patent Application Publication
(10) Pub. No.: US 2024/0192196 A1
(43) Pub. Date: Jun. 13, 2024

(54) NOVEL CORONAVIRUS SARS-CoV-2 SAFE REPLICON SYSTEM AND USE THEREOF

(71) Applicant: SUN YAT-SEN UNIVERSITY, Guangzhou, Guangdong (CN)

(72) Inventors: Hui ZHANG, Guangzhou, Guangdong (CN); Yuewen LUO, Guangzhou, Guangdong (CN)

(21) Appl. No.: 18/021,348

(22) PCT Filed: Sep. 30, 2020
(86) PCT No.: PCT/CN2020/119544

§ 371 Date: Feb. 14, 2023

(30) Foreign Application Priority Data
Aug. 14, 2020 (CN) 202010818896.X

(51) Int. Cl. G01N 33/50; C07K 14/005; C12N 15/52; C12Q 1/66

(52) U.S. Cl. CPC G01N 33/5023; C07K 14/005; C12N 15/52; C12Q 1/66; C12N 2770/20022; C12N 2770/20052; G01N 2333/90241; G01N 2500/10

(57) ABSTRACT

The present disclosure discloses a novel coronavirus SARS-COV-2 safe replicon system and use thereof in screening an anti-SARS-COV-2 drug. The safe replicon system specifically comprises a nucleic acid sequence encoding a novel coronavirus SARS-COV-2 non-structural protein; and nucleic acid sequences of 5′ UTR and 3′ UTR of a novel coronavirus SARS-COV-2, a transcription regulatory region on which the novel coronavirus SARS-COV-2 non-structural protein can act, and a reporter gene. With the SARS-COV-2 safe replicon system, high-throughput screening of anti-SARS-COV-2 drugs and pharmacologic verification of drugs can be carried out independent of a biosafety level 3 laboratory, and the operation is simple and convenient.

### CROSS-REFERENCE TO RELATED APPLICATION

The present application is a national phase entry under 35 USC § 371 of International Application PCT/CN2020/119544, filed Sep. 30, 2020, which claims the benefit of and priority to Chinese Patent Application No. 202010818896.X, filed Aug. 14, 2020, the entire disclosures of which are incorporated herein by reference.

### INCORPORATION BY REFERENCE

This application includes a sequence listing in computer readable form (a “txt” file) that is submitted herewith on ASCII text file named P23GZINW00010US_ST25.txt, created on Aug. 21, 2023 and 69,974 bytes in size. This sequence listing is incorporated by reference herein.

### TECHNICAL FIELD

The present disclosure belongs to the field of biotechnology, and more particularly relates to a novel coronavirus SARS-COV-2 safe replicon system and use thereof.

### BACKGROUND

As of Jul. 23, 2020, novel coronavirus SARS-COV-2 had infected more than 15 million people worldwide and killed more than 140,000 people. However, there are currently very limited clinical therapeutic drugs applicable to SARS-COV-2 infection. For biosafety reasons, drug development and screening against wild-type SARS-COV-2 can only be carried out in biosafety level 3 laboratories (P3 laboratories), which greatly limits the development of antiviral drugs against SARS-COV-2.

Previous studies have shown that a safe replicon system constructed by inserting E protein-deleted coronavirus genome into a Bacterial Artificial Chromosome (BAC) can simulate the replication of coronaviruses. This system has been applied to drug verification and drug screening against SARS-COV. However, the system is based on BAC plasmid. BAC plasmid, which has a relatively large molecular weight and is unstable, cannot reach an ideal expression level after transduction into cells and is also time-consuming and laborious to operate.

Therefore, it is urgent to develop a tool that can simulate SARS-COV-2 virus replication and can be simply operated in low-level biosafety laboratories.

### SUMMARY

The object of a first aspect of the present disclosure is to provide a novel SARS-COV-2 safe replicon structure which can make up for the blank of novel coronavirus SARS-COV-2 safe replicons while overcoming the deficiency of BAC replicon systems.

The object of a second aspect of the present disclosure is to provide a novel coronavirus SARS-COV-2 safe replicon system comprising the above-mentioned replicon structure.

The object of a third aspect of the present disclosure is to provide a packaging cell comprising the above-mentioned replicon structure or replicon system.

The object of a fourth aspect of the present disclosure is to provide use of the above-mentioned novel coronavirus SARS-COV-2 safe replicon structure, replicon system or packaging cell in drug detection or drug screening against novel coronavirus SARS-COV-2.

The object of a fifth aspect of the present disclosure is to provide a method for screening an anti-novel coronavirus SARS-COV-2 drug.

The object of a sixth aspect of the present disclosure is to provide a kit for screening an anti-novel coronavirus SARS-COV-2 drug.

The object of a seventh aspect of the present disclosure is to provide a system for screening an anti-novel coronavirus SARS-COV-2 drug.

The object of an eighth aspect of the present disclosure is to provide a novel coronavirus SARS-COV-2 molecular epidemiological monitoring system.

The following technical solutions are used in the present disclosure:

In the first aspect of the present disclosure, provided is a novel coronavirus SARS-CoV-2 replicon structure, comprising the following nucleic acid sequences:
(I) a nucleic acid sequence encoding a novel coronavirus SARS-COV-2 non-structural protein; and
(II) nucleic acid sequences of 5′ UTR and 3′ UTR of the novel coronavirus SARS-COV-2, a transcription regulatory region on which the novel coronavirus SARS-COV-2 non-structural protein can act, and a reporter gene.

Preferably, according to the replicon structure of the first aspect of the present disclosure, the non-structural protein is at least one of novel coronavirus SARS-COV-2 protein nsps 1-16.

Preferably, according to the replicon structure of the first aspect of the present disclosure, the transcription regulatory region is at least one of transcription regulatory regions (TRSs) of S, ORF3a, M, ORF7a, ORF8 or N genes of novel coronavirus SARS-COV-2.

Furthermore, a core sequence (AAACGAAC) of the transcription regulatory region (TRS) alone, or a combination of the core sequence (AAACGAAC) of the transcription regulatory region and another sequence, also falls within the scope of protection.

Furthermore, according to the replicon structure of the first aspect of the present disclosure, the transcription regulatory region is connected upstream of the reporter gene.

Furthermore, the replicon structure of the first aspect of the present disclosure further comprises a nucleic acid sequence of an additional reporter gene as a reference.

Moreover, according to the replicon structure of the first aspect of the present disclosure, the additional reporter gene as the reference is connected to a stop codon and located upstream of the transcription regulatory region.

Preferably, according to the replicon structure of the first aspect of the present disclosure, the nucleic acid is DNA or RNA, preferably antisense RNA.

In the second aspect of the present disclosure, provided is a novel coronavirus SARS-CoV-2 replicon system, comprising an expression vector in which the replicon structure according to the first aspect of the present disclosure is inserted.

Preferably, the replicon system according to the second aspect of the present disclosure comprises the following two expression vectors comprising:
(i) a nucleic acid sequence encoding a novel coronavirus SARS-COV-2 non-structural protein; and
(ii) nucleic acid sequences of 5′ UTR and 3′ UTR of a novel coronavirus SARS-COV-2, a transcription regulatory region on which the novel coronavirus SARS-COV-2 non-structural protein can act, and a reporter gene.

More preferably, according to the replicon system of the second aspect of the present disclosure, nucleic acid sequences of the 5′ UTR of novel coronavirus SARS-COV-2, the transcription regulatory region on which the novel coronavirus SARS-COV-2 non-structural protein can act, the reporter gene, and the 3′ UTR of novel coronavirus SARS-COV-2 are inserted in expression vector (ii) in order.

Further preferably, according to the replicon system of the second aspect of the present disclosure, nucleic acid sequences of the 5′ UTR of novel coronavirus SARS-COV-2, a reporter gene A, the transcription regulatory region on which the novel coronavirus SARS-COV-2 non-structural protein can act, a reporter gene B, and the 3′ UTR of novel coronavirus SARS-COV-2 are inserted in expression vector (ii) in order, wherein the reporter gene A is different from the reporter gene B.

More preferably, the reporter gene A is a nucleic acid sequence of fluorescent protein; and the reporter gene B is a nucleic acid sequence encoding luciferase.

Moreover, according to the replicon system of the second aspect of the present disclosure, a nucleic acid sequence of a ribosome entry site (IRES) is further connected between the 5′ UTR of novel coronavirus SARS-COV-2 and the reporter gene A.

Moreover, according to the replicon system of the second aspect of the present disclosure, a translation stop codon, preferably four stop codons, is inserted at an end of reporter gene A.

Specifically, according to the replicon system of the second aspect of the present disclosure, nucleic acid sequences of the 5′ UTR of novel coronavirus SARS-COV-2, the reporter gene A, the transcription regulatory region on which the novel coronavirus SARS-COV-2 non-structural protein can act, the reporter gene B, and the 3′ UTR of novel coronavirus SARS-COV-2 are inserted in expression vector (ii) in order, wherein the reporter gene A is a nucleic acid sequence of fluorescent protein; and the reporter gene B is a nucleic acid sequence encoding luciferase.

Furthermore, the transcription regulatory region is a transcription regulatory region upstream of S, ORF3a, M, ORF7a, ORF8, or N genes of novel coronavirus SARS-COV-2.

Moreover, the nucleotide sequence of the transcription regulatory region for S protein (S-TRS) is shown in SEQ ID NO:20; the nucleic acid sequence of the transcription regulatory region for ORF3a protein (ORF3a-TRS) is shown in SEQ ID NO: 21: the nucleic acid sequence of the transcription regulatory region for protein M (M-TRS) is shown in SEQ ID NO:22: the nucleic acid sequence of the transcription regulatory region for ORF7a protein (ORF7a-TRS) is shown in SEQ ID NO: 23: the nucleic acid sequence of the transcription regulatory region for ORF8 protein (ORF8-TRS) is shown in SEQ ID NO: 24; and the nucleic acid sequence of the transcription regulatory region for N protein (N-TRS) is shown in SEQ ID NO: 25.

The nucleotide sequence of the 5′ UTR of novel coronavirus SARS-COV-2 is shown in SEQ ID NO: 26.

The nucleotide sequence of the 3′ UTR of novel coronavirus SARS-COV-2 is shown in SEQ ID NO: 27.

The nucleotide sequence of the inserted ribosome entry site (IRES) is preferably shown in SEQ ID NO: 28.

The nucleotide sequence of the inserted four stop codons is preferably shown in SEQ ID NO: 29.

More specifically, the nucleotide sequence of ps2V in expression vector (ii) is shown in SEQ ID NO: 30.

Preferably, according to the replicon system of the second aspect of the present disclosure, the encoded novel coronavirus SARS-COV-2 non-structural protein is novel coronavirus SARS-COV-2 protein nsps 1-16.

More preferably, according to the replicon system of the second aspect of the present disclosure, the reporter gene A is a nucleic acid sequence of fluorescent protein; and the reporter gene B is a nucleic acid sequence encoding luciferase. Expression vector (i) comprises three expression vectors, in which nucleic acid sequences encoding one or more of novel coronavirus SARS-COV-2 protein nsps 1-16 are respectively inserted.

Further preferably, the nucleic acid sequences of protein nsps 1-16 are codon-optimized.

More specifically, after codon optimization, the nucleotide sequence of nsp 1 is shown in SEQ ID NO: 1: the nucleotide sequence of nsp 2 is shown in SEQ ID NO: 2: the nucleotide sequence of nsp 3 is shown in SEQ ID NO: 3: the nucleotide sequence of nsp 4 is shown in SEQ ID NO: 4: the nucleotide sequence of nsp 5 is shown in SEQ ID NO: 5: the nucleotide sequence of nsp 6 is shown in SEQ ID NO: 6: the nucleotide sequence of nsp 7 is shown in SEQ ID NO: 7: the nucleotide sequence of nsp 8 is shown in SEQ ID NO: 8; the nucleotide sequence of nsp 9 is shown in SEQ ID NO: 9; the nucleotide sequence of nsp 10 is shown in SEQ ID NO: 10; the nucleotide sequence of nsp 11 is shown in SEQ ID NO: 11: the nucleotide sequence of nsp 12 is shown in SEQ ID NO: 12; the nucleotide sequence of nsp 13 is shown in SEQ ID NO: 13; the nucleotide sequence of nsp 14 is shown in SEQ ID NO: 14; the nucleotide sequence of nsp 15 is shown in SEQ ID NO: 15; and the nucleotide sequence of nsp 16 is shown in SEQ ID NO: 16.

Further preferably, according to the replicon system of the second aspect of the present disclosure, a nucleic acid sequence encoding novel coronavirus SARS-COV-2 protein nsps 1-4, a nucleic acid sequence encoding novel coronavirus SARS-COV-2 protein nsps 5-11, and a nucleic acid sequence encoding novel coronavirus SARS-COV-2 protein nsps 12-16 are respectively inserted in the three expression vectors.

Moreover, according to the replicon system of the second aspect of the present disclosure, the nucleic acid sequences are codon-optimized.

Specifically, according to the replicon system of the second aspect of the present disclosure, expression vector (i) comprises three expression vectors, in which three segments of nucleic acid sequences, i.e., ps2AN, ps2AC, and ps2B, are inserted respectively.
ps2AN comprises a nucleic acid sequence encoding novel coronavirus SARS-COV-2 protein nsps 1-4;
ps2AC comprises a nucleic acid sequence encoding novel coronavirus SARS-COV-2 protein nsps 5-11; and
ps2B comprises a nucleic acid sequence encoding novel coronavirus SARS-COV-2 protein nsps 12-16.

Moreover, the nucleotide sequence of ps2AN is shown in SEQ ID NO: 17; the nucleotide sequence of ps2AC is shown in SEQ ID NO: 18; and the nucleotide sequence of ps2B is shown in SEQ ID NO: 19.

Preferably, according to the replicon system of the second aspect of the present disclosure, the expression vector is preferably but not limited to pcDNA3.1 plasmid.

More preferably, the ratio of the plasmids respectively comprising ps2AN, ps2AC, ps2B and ps2V is (0.01-1 μg): (0.01-1 μg): (0.01-1 μg): (0.01-1 μg).

In the third aspect of the present disclosure, provided is a packaging cell comprising the replicon structure of the first aspect of the present disclosure or the replicon system of the second aspect of the present disclosure.

Preferably, according to the packaging cell of the third aspect of the present disclosure, the cell is a human-derived cell.

More preferably, according to the packaging cell of the third aspect of the present disclosure, the cell is preferably but not limited to an HEK293T cell.

Preferably, according to the packaging cell of the third aspect of the present disclosure, the replicon structure or replicon system is codon-optimized.

Furthermore, the replicon structure or replicon system is transfected into a cell, to form a packaging cell.

Moreover, during transfection, the ratio of the plasmids respectively comprising ps2AN, ps2AC, ps2B and ps2V is (0.01-1 μg): (0.01-1 μg): (0.01-1 μg): (0.01-1 μg).

The ratio of the plasmids is (0.01-1 μg): (0.01-1 μg): (0.01-1 μg): (0.01-1 μg) on a concentration basis.

In the fourth aspect of the present disclosure, provided is use of the replicon structure of the first aspect of the present disclosure, the replicon system of the second aspect of the present disclosure, or the packaging cell of the third aspect of the present disclosure in drug detection or drug screening against novel coronavirus SARS-COV-2.

In the fifth aspect of the present disclosure, provided is a method for screening an anti-novel coronavirus SARS-COV-2 drug, comprising adding a drug to be tested to an expression system comprising the replicon structure of the first aspect of the present disclosure, the replicon system of the second aspect of the present disclosure or the packaging cell of the third aspect of the present disclosure to detect the differential expression of a reporter gene and evaluate an anti-novel coronavirus SARS-COV-2 effect of the drug to be tested.

In the sixth aspect of the present disclosure, provided is a kit for screening an anti-novel coronavirus SARS-COV-2 drug, comprising the replicon structure of the first aspect of the present disclosure, the replicon system of the second aspect of the present disclosure, or the packaging cell of the third aspect of the present disclosure.

In the seventh aspect of the present disclosure, provided is a system for screening an anti-novel coronavirus SARS-COV-2 drug, comprising the replicon structure of the first aspect of the present disclosure, the replicon system of the second aspect of the present disclosure, or the packaging cell of the third aspect of the present disclosure.

Furthermore, the drug screening system according to the seventh aspect of the present disclosure further comprises a luciferase detection device.

Preferably, the drug screening system further comprises a fluorescent protein detection device.

Preferably, the drug screening system further comprises a fully automatic robotic arm drug screening platform.

In the eighth aspect of the present disclosure, provided is a novel coronavirus SARS-CoV-2 molecular epidemiological monitoring system, comprising the replicon structure of the first aspect of the present disclosure, the replicon system of the second aspect of the present disclosure, or the packaging cell of the third aspect of the present disclosure.

The novel coronavirus SARS-COV-2 molecular epidemiological monitoring system according to the eighth aspect of the present disclosure utilizes the replicon system to monitor an effect of a mutation produced in SARS-COV-2 during an epidemic on SARS-COV-2 virus replication.

The present disclosure has the following beneficial effects.

The present disclosure provides a novel coronavirus SARS-COV-2 safe replicon structure, a novel coronavirus SARS-COV-2 safe replicon system, and a packaging cell thereof which can make up for the blank of novel coronavirus SARS-COV-2 safe replicons while overcoming the technical deficiency of BAC replicon systems. The molecule necessary for the synthesis of SARS-COV-2 RNA is artificially split, optimized for nucleotide sequence, so that four plasmids are used for co-expression, which destroys the original SARS-COV-2 sequence and provides safer operation, without the need for operation in a high-level biosafety laboratory.

The novel coronavirus SARS-COV-2 safe replicon system constructed by the present disclosure can highly simulate the response of wild-type SARS-COV-2 to a drug.

The present disclosure further provides a method for screening an anti-novel coronavirus SARS-COV-2 drug, a corresponding kit, and a detection system. The present disclosure provides the possibility of screening an anti-novel coronavirus SARS-COV-2 drug in laboratories below biosafety level 3, greatly promotes the research and screening of anti-novel coronavirus SARS-COV-2 drugs, and has broad application prospects.

The novel coronavirus SARS-COV-2 safe replicon system constructed by the present disclosure can highly simulate the replication characteristics of wild-type SARS-COV-2. Another potential application of the present disclosure is that point mutation can be artificially performed in the replicon system according to the mutation characteristics of epidemic strains, and the effect of the epidemic mutation on virus replication can then be detected and evaluated, which is of positive significance for molecular epidemiological monitoring of novel coronavirus SARS-COV-2.

### BRIEF DESCRIPTION OF DRAWINGS

FIG. 1 is a schematic diagram of the composition of the genome of novel coronavirus SARS-COV-2.

FIG. 2 is a schematic diagram of the functions of novel coronavirus SARS-COV-2 protein nsps 1-16.

FIG. 3 is a schematic diagram of the viral structure of novel coronavirus SARS-COV-2.

FIG. 4 is a schematic diagram of the replication process of novel coronavirus SARS-CoV-2.

FIG. 5 is a schematic structural diagram of molecules of constructed ps2V, ps2AN, ps2AC and ps2B vectors.

FIG. 6 shows the working principle of a novel coronavirus SARS-COV-2 safe replicon system.

FIG. 7 is a plasmid map of pcDNA3.1.

FIG. 8A shows the expression of GFP after transfection with ps2V alone, and FIG. 8B shows the expression of GFP after transfection with ps2V mixed with ps2AN, ps2AC, and ps2B plasmids.

FIG. 9 shows the luciferase activity in HEK 293T cells transfected with ps2V, ps2AN, ps2AC and ps2B vectors over time.

FIG. 10 shows the inhibitory effect of Remdesivir verified by the novel coronavirus SARS-COV-2 safe replicon system.

FIG. 11 shows the inhibitory effect of Lopinavir verified by the novel coronavirus SARS-COV-2 safe replicon system.

FIG. 12 shows the inhibitory effect of Ritonavir verified by the novel coronavirus SARS-COV-2 safe replicon system.

FIG. 13A shows the inhibitory effect of M01 on viral RNA replication detected by the novel coronavirus SARS-COV-2 safe replicon system: FIG. 13B shows the inhibitory effect of A01 on viral RNA replication detected by the novel coronavirus SARS-COV-2 safe replicon system; and FIG. 13C shows the inhibitory effect of R01 on viral RNA replication detected by the novel coronavirus SARS-COV-2 safe replicon system.

FIG. 14A shows the inhibitory effects of M01 on wild-type SARS-COV-2: FIG. 14B shows the inhibitory effects of A01 on wild-type SARS-COV-2; and FIG. 14C shows the inhibitory effects of R01 on wild-type SARS-COV-2.

FIG. 15 is a schematic diagram of the study on the evolution of virus.

FIG. 16 shows the luciferase detection results of 5′ UTR_241 T_ps2V and 5′ UTR_241 C_ps2V.

### DETAILED DESCRIPTION

In order to understand the technical content of the present disclosure more clearly, the following examples are particularly given in conjunction with the attached drawings for detailed description. It should be understood that these examples are only used to illustrate the present disclosure, rather than limiting the scope of the present disclosure. The experimental methods without specific conditions indicated in the following examples usually follow conventional conditions, such as those in Sambrook et al., Molecular Cloning: A Laboratory Manual (New York: Cold Spring Harbor Laboratory Press, 1989), or those suggested by the manufacturer. Various conventional chemical reagents used in the examples are all commercially available products.

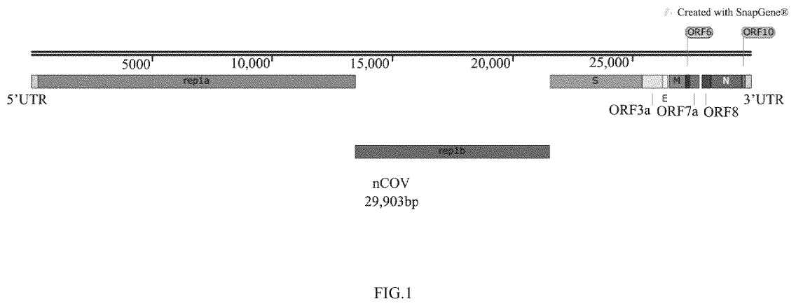

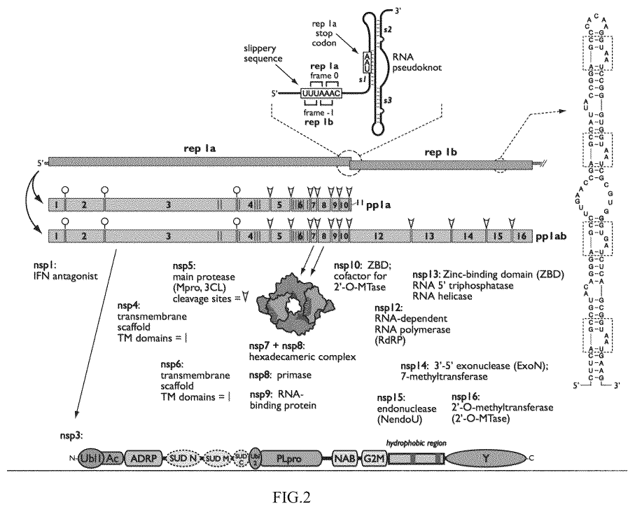

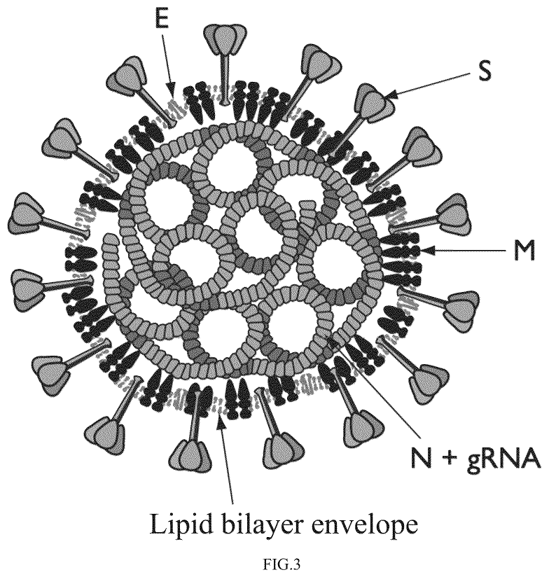

The genome of novel coronavirus SARS-COV-2 is shown in FIG. 1. 5′ UTR and 3′ UTR are non-coding regions and are associated with viral replication and transcription. The genomes rep1a and rep1b encode nsps 1-16, and proteins nsps 1-16 mature to form a viral transcriptase/replicase complex. Among them, the protease expressed by nsp 3 can cleave protein nsps 1-4, the protease expressed by nsp5 can cleave protein nsps 5-16, and the schematic diagram of the functions of nsps 1-16 is shown in FIG. 2. Furthermore, in addition to 5′ UTR, 3′ UTR, rep1a and rep1b, the genome of novel coronavirus further comprises sequences encoding N, S, E and M proteins (see FIG. 1). N, S, E and M encode structural proteins of the virus to form a virion (as shown in FIG. 3), and the remaining ORF3a, ORF7a, ORF8, ORF6, and ORF10 encode accessory proteins, whose functions are currently unclear.

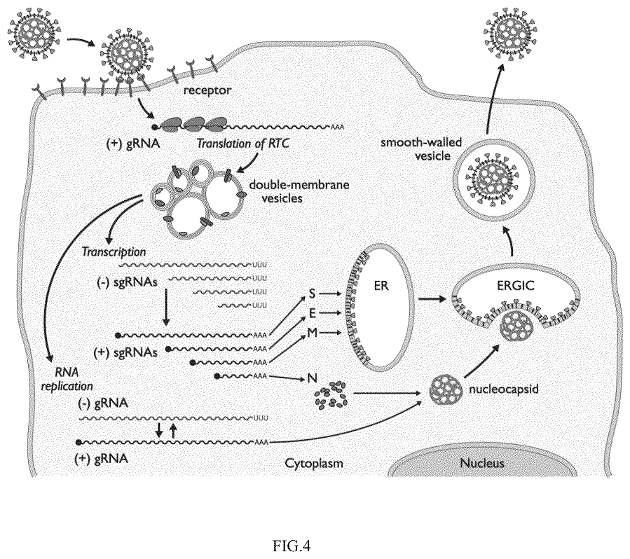

After novel coronavirus SARS-COV-2 enters a cell via ACE2 receptor:
1. rep1a and rep1b are firstly transcribed and translated into protein nsps 1-16 to form complexes (double-membrane vesicles), and the virus can only conduct RNAs synthesis (RNA replication and transcription) in the complexes.
2. The viral RNAs undergo two biological processes in the above-mentioned complexes (double-membrane vesicles):
a. transcription: i.e., the synthesis of viral sub-genomic RNAs (different from small segments of RNAs/sub-genomic RNAs in the viral genome), the process of which depends on the participation of protein nsps 1-16, and on 5′ UTR sequence, 3′ UTR sequence, and transcription regulatory region (TRS) sequences in the viral genome. After the newly transcribed sub-genomic RNAs, which are negative strands, are replicated and transformed into positive strands, these sub-genomic RNAs express structural proteins N, S, E, and M, which wrap the genomic RNA and exit the cell to form a virion.
b. Replication: genomic RNA and sub-genomic RNAs can be replicated in double-membrane vesicles, i.e., mutual transformation from negative strand RNA to positive strand RNA, to increase the number of RNA copies, see FIG. 4 for the schematic diagram of the replication process of novel coronavirus SARS-COV-2.

The original sequence of novel coronavirus SARS-COV-2 is based on the sequence of SARS-COV-2 Wuhan-Hu-1 (Genbank: NC_045512.2).

### Example 1 Construction of Replicon

Based on the composition of the genome of novel coronavirus and the principle and process of viral RNA synthesis (replication and transcription process), a novel coronavirus SARS-COV-2 safe replicon, including the following two expression structures is creatively constructed:
(I) a nucleic acid sequence encoding a novel coronavirus SARS-COV-2 non-structural protein; and
(II) nucleic acid sequences of 5′ UTR and 3′ UTR of a novel coronavirus SARS-COV-2, a transcription regulatory region on which the novel coronavirus SARS-COV-2 non-structural protein can act, and a reporter gene.

The expression structure comprising a nucleic acid sequence encoding the novel coronavirus SARS-COV-2 non-structural protein in (I) was an expression vector encoding the sequences of protein nsps 1-16.

In the genome of novel coronavirus, the sequences of rep1a and rep1b, totaling about 20000 bp, accounted for about ⅔ of the viral genome. Considering the efficiency of transfection and expression, as well as the function of each of protein nsps 1-16 in the transcription complex, the nucleotide sequences encoding protein neps 1-16 were codon-optimized and inserted into three expression vectors, respectively, named ps2AN, ps2AC, and ps2B respectively.

After codon optimization, the nucleotide sequence of nsp 1 was
shown in SEQ ID NO: 1:
(SEQ ID NO: 1)
ATGGAGTCCCTGGTGCCCGGCTTCAACGAGAAGACCCACGTGCAGCTGTCTC
TGCCTGTGCTGCAGGTGAGGGATGTGCTGGTGCGCGGCTTTGGCGACTCCGTCGA
GGAGGTGCTGTCTGAGGCCAGGCAGCACCTGAAGGACGGAACCTGCGGACTGGT
GGAGGTGGAGAAGGGCGTGCTGCCACAGCTGGAGCAGCCTTACGTGTTCATCAAG
AGGTCCGATGCAAGGACAGCACCACACGGACACGTGATGGTGGAGCTGGTGGCC
GAGCTGGAGGGCATCCAGTATGGCCGCTCTGGAGAGACCCTGGGCGTGCTGGTGC
CACACGTGGGAGAGATCCCAGTGGCCTATCGGAAGGTGCTGCTGAGAAAGAACG
GCAATAAGGGAGCAGGAGGACACTCTTACGGAGCAGACCTGAAGAGCTTCGATCT
GGGCGACGAGCTGGGCACCGATCCTTATGAGGACTTTCAGGAGAACTGGAATACA
AAGCACAGCTCCGGCGTGACCCGGGAGCTGATGAGAGAGCTGAACGGCGGC.
The nucleotide sequence of nsp 2 was shown in
SEQ ID NO: 2:
(SEQ ID NO: 2)
GCCTACACCAGATATGTGGATAACAATTTCTGCGGACCAGACGGATACCCCCT
GGAGTGTATCAAGGATCTGCTGGCCAGAGCAGGCAAGGCCTCCTGCACCCTGTCT
GAGCAGCTGGACTTCATCGACACAAAGCGGGGCGTGTATTGCTGTAGAGAGCACG
AGCACGAGATCGCCTGGTATACCGAGCGGTCCGAGAAGTCTTACGAGCTGCAGAC
ACCATTCGAGATCAAGCTGGCCAAGAAGTTCGACACCTTCAACGGCGAGTGTCCA
AACTTCGTGTTTCCCCTGAATAGCATCATCAAGACCATCCAGCCCAGAGTGGAGAA
GAAGAAGCTGGATGGCTTTATGGGCAGGATCCGCAGCGTGTACCCTGTGGCCTCCC
CAAACGAGTGCAATCAGATGTGCCTGTCCACACTGATGAAGTGCGATCACTGTGG
CGAGACCTCTTGGCAGACAGGCGACTTCGTGAAGGCCACCTGCGAGTTTTGTGGC
ACCGAGAACCTGACAAAGGAGGGCGCCACCACATGCGGCTATCTGCCTCAGAATG
CCGTGGTGAAGATCTACTGCCCAGCCTGTCACAACTCCGAAGTGGGACCAGAGCA
CTCTCTGGCCGAGTACCACAATGAGTCCGGCCTGAAGACAATCCTGAGGAAGGGA
GGAAGGACCATCGCCTTCGGCGGATGCGTGTTTTCTTATGTGGGCTGCCACAACAA
GTGTGCATACTGGGTGCCAAGGGCCAGCGCCAATATCGGCTGTAACCACACCGGA
GTGGTGGGAGAGGGATCCGAGGGCCTGAACGATAATCTGCTGGAGATCCTGCAGA
AGGAGAAGGTGAACATCAATATCGTGGGCGACTTCAAGCTGAACGAGGAGATCGC
CATCATCCTGGCCTCCTTCTCTGCCAGCACATCCGCCTTTGTGGAGACCGTGAAGG
GCCTGGACTACAAGGCCTTCAAGCAGATCGTGGAGAGCTGCGGCAACTTCAAGGT
GACCAAGGGCAAGGCCAAGAAGGGCGCCTGGAACATCGGCGAGCAGAAGAGCAT
CCTGTCCCCTCTGTATGCCTTCGCCAGCGAGGCAGCAAGGGTGGTGAGATCTATCT
TTAGCCGGACCCTGGAGACAGCCCAGAATTCCGTGAGAGTGCTGCAGAAGGCCGC
CATCACCATCCTGGATGGCATCTCCCAGTACTCTCTGAGGCTGATCGATGCCATGAT
GTTCACCTCCGACCTGGCCACAAACAATCTGGTGGTCATGGCCTACATCACCGGCG
GCGTGGTGCAGCTGACCTCTCAGTGGCTGACAAACATCTTTGGCACCGTGTATGA
GAAGCTGAAGCCAGTGCTGGATTGGCTGGAGGAGAAGTTCAAGGAGGGCGTGGA
GTTTCTGCGCGACGGCTGGGAGATCGTGAAGTTCATCAGCACCTGCGCATGTGAG
ATCGTGGGAGGACAGATCGTGACCTGTGCCAAGGAGATCAAGGAGTCCGTGCAGA
CATTCTTTAAGCTGGTGAACAAGTTCCTGGCCCTGTGCGCCGACTCTATCATCATCG
GCGGCGCCAAGCTGAAGGCCCTGAACCTGGGCGAGACCTTTGTGACACACAGCA
AGGGCCTGTACAGGAAGTGCGTGAAGTCCCGCGAGGAGACCGGACTGCTGATGC
CCCTGAAGGCACCTAAGGAGATCATCTTCCTGGAGGGCGAGACCCTGCCCACAGA
GGTGCTGACAGAGGAGGTGGTGCTGAAGACCGGCGACCTGCAGCCACTGGAGCA
GCCCACCAGCGAGGCAGTGGAGGCACCTCTGGTGGGCACACCAGTGTGCATCAAT
GGCCTGATGCTGCTGGAGATCAAGGATACCGAGAAGTACTGTGCCCTGGCCCCTA
ACATGATGGTGACAAACAATACCTTCACACTGAAGGGCGGC.
The nucleotide sequence of nsp 3 was shown in
SEQ ID NO: 3:
(SEQ ID NO: 3)
GCCCCAACCAAGGTGACATTTGGCGACGATACCGTGATCGAGGTGCAGGGCT
ACAAGTCTGTGAATATCACATTCGAGCTGGATGAGAGAATCGACAAGGTGCTGAA
CGAGAAGTGCAGCGCCTATACAGTGGAGCTGGGCACCGAGGTGAACGAGTTTGCC
TGCGTGGTGGCCGACGCCGTGATCAAGACCCTGCAGCCAGTGTCCGAGCTGCTGA
CACCCCTGGGCATCGATCTGGACGAGTGGTCTATGGCCACCTACTATCTGTTCGAC
GAGAGCGGCGAGTTTAAGCTGGCCTCCCACATGTACTGCTCTTTCTATCCCCCTGA
TGAAGACGAGGAGGAGGGCGATTGCGAGGAGGAGGAGTTTGAGCCCAGCACACA
GTACGAGTATGGCACCGAGGACGATTACCAGGGCAAGCCACTGGAGTTCGGAGCC
ACCTCCGCCGCCCTGCAGCCAGAGGAGGAGCAGGAGGAGGATTGGCTGGACGAT
GACTCCCAGCAGACCGTGGGCCAGCAGGATGGCTCTGAGGACAATCAGACCACA
ACCATCCAGACAATCGTGGAGGTGCAGCCTCAGCTGGAGATGGAGCTGACCCCAG
TGGTGCAGACCATCGAGGTGAACTCTTTCAGCGGCTATCTGAAGCTGACAGATAAC
GTGTACATCAAGAACGCCGACATTGTGGAGGAGGCCAAGAAGGTGAAGCCTACCG
TGGTGGTGAACGCCGCCAACGTGTACCTGAAGCACGGAGGAGGAGTGGCAGGCG
CCCTGAACAAGGCCACCAACAATGCCATGCAGGTGGAGAGCGATGACTATATCGC
CACAAATGGACCCCTGAAGGTCGGAGGAAGCTGCGTGCTGTCCGGACACAACCT
GGCCAAGCACTGTCTGCACGTGGTGGGCCCTAACGTGAATAAGGGCGAGGACATC
CAGCTGCTGAAGTCCGCCTACGAGAACTTCAATCAGCACGAGGTGCTGCTGGCCC
CTCTGCTGAGCGCCGGCATCTTTGGCGCCGATCCAATCCACTCCCTGAGGGTGTGC
GTGGACACCGTGCGCACAAACGTGTACCTGGCCGTGTTCGATAAGAACCTGTACG
ACAAGCTGGTGTCTAGCTTTCTGGAGATGAAGAGCGAGAAGCAGGTGGAGCAGA
AGATCGCCGAGATCCCTAAGGAGGAGGTGAAGCCATTCATCACCGAGAGCAAGCC
TTCCGTGGAGCAGAGGAAGCAGGATGACAAGAAGATCAAGGCCTGCGTGGAGGA
GGTGACAACCACACTGGAGGAGACCAAGTTCCTGACAGAGAACCTGCTGCTGTA
CATCGATATCAACGGCAATCTGCACCCAGACAGCGCCACACTGGTGTCCGATATCG
ACATCACCTTTCTGAAGAAGGATGCCCCATATATCGTGGGCGACGTGGTGCAGGAG
GGCGTGCTGACAGCCGTGGTCATCCCCACCAAGAAGGCCGGCGGCACCACAGAG
ATGCTGGCCAAGGCCCTGCGCAAGGTGCCTACCGACAATTACATCACCACATATCC
AGGCCAGGGCCTGAACGGCTATACCGTGGAGGAGGCCAAGACCGTGCTGAAGAA
GTGCAAGAGCGCCTTCTACATCCTGCCTTCTATCATCAGCAATGAGAAGCAGGAGA
TCCTGGGCACCGTGTCCTGGAACCTGAGGGAGATGCTGGCCCACGCCGAGGAGAC
ACGCAAGCTGATGCCCGTGTGCGTGGAGACAAAGGCCATCGTGAGCACCATCCAG
CGGAAGTATAAGGGCATCAAGATCCAGGAGGGAGTGGTGGACTACGGAGCAAGAT
TCTACTTTTATACCTCTAAGACCACAGTGGCCAGCCTGATCAACACACTGAATGATC
TGAACGAGACCCTGGTGACAATGCCCCTGGGCTATGTGACCCACGGCCTGAATCT
GGAGGAGGCCGCCAGGTACATGCGCTCCCTGAAGGTGCCAGCAACCGTGAGCGT
GAGCTCTCCTGACGCCGTGACAGCCTACAACGGCTATCTGACAAGCTCCTCTAAG
ACCCCAGAGGAGCACTTCATCGAGACCATCTCTCTGGCCGGCAGCTATAAGGATTG
GTCCTACTCTGGCCAGTCCACACAGCTGGGCATCGAGTTTCTGAAGAGGGGCGAC
AAGAGCGTGTACTATACCAGCAATCCCACCACATTCCACCTGGATGGCGAAGTGAT
CACCTTCGACAACCTGAAGACCCTGCTGAGCCTGCGGGAGGTGAGAACCATCAAG
GTGTTCACCACAGTGGATAACATCAATCTGCACACACAGGTGGTGGACATGTCCAT
GACCTATGGCCAGCAGTTTGGCCCAACATACCTGGATGGCGCCGACGTGACCAAG
ATCAAGCCCCACAATAGCCACGAGGGCAAGACATTCTACGTGCTGCCTAATGCCAC
CAACTTTTCCCTGCTGAAGCAGGCAGGCGACGTGGAGGAGAACCCAGGACCAGA
TGACACCCTGAGGGTGGAGGCCTTCGAGTACTATCACACCACAGATCCTAGCTTTC
TGGGCCGCTATATGTCCGCCCTGAATCACACCAAGAAGTGGAAGTACCCACAGGT
GAACGGCCTGACAAGCATCAAGTGGGCCGACAACAATTGCTACCTGGCCACCGCC
CTGCTGACACTGCAGCAGATCGAGCTGAAGTTCAACCCACCCGCCCTGCAGGATG
CATACTATAGGGCAAGAGCAGGAGAGGCAGCCAATTTTTGCGCCCTGATCCTGGCC
TATTGTAACAAGACCGTGGGAGAGCTGGGCGATGTGCGGGAGACAATGAGCTACC
TGTTCCAGCACGCCAATCTGGACTCCTGCAAGAGAGTGCTGAACGTGGTGTGCAA
GACATGTGGCCAGCAGCAGACCACACTGAAGGGCGTGGAGGCCGTGATGTATATG
GGCACCCTGAGCTACGAGCAGTTTAAGAAGGGCGTGCAGATCCCCTGCACATGTG
GCAAGCAGGCCACCAAGTACCTGGTGCAGCAGGAGTCCCCTTTCGTGATGATGTC
TGCCCCTCCAGCCCAGTATGAGCTGAAGCACGGCACCTTTACATGCGCCTCTGAGT
ACACCGGCAATTATCAGTGTGGCCACTATAAGCACATCACCAGCAAGGAGACACT
GTACTGCATCGATGGCGCCCTGCTGACCAAGAGCTCCGAGTACAAGGGCCCCATC
ACAGACGTGTTCTATAAGGAGAATTCTTACACCACAACCATCGCCACCAACTTTAG
CCTGCTGAAGCAGGCCGGCGATGTGGAGGAGAACCCTGGACCAAAGCCCGTGAC
CTATAAGCTGGACGGCGTGGTGTGCACAGAGATCGATCCTAAGCTGGACAACTACT
ACAAGAAGGATAACTCTTATTTCACCGAGCAGCCCATCGACCTGGTGCCTAATCAG
CCTTACCCAAACGCCAGCTTCGATAATTTCAAGTTCGTGTGCGACAATATCAAGTTT
GCCGATGACCTGAACCAGCTGACCGGATACAAGAAGCCAGCCAGCCGGGAGCTG
AAGGTGACATTCTTTCCTGATCTGAACGGCGACGTGGTGGCCATCGACTACAAGC
ACTATACACCTTCCTTCAAGAAGGGCGCCAAGCTGCTGCACAAGCCAATCGTGTG
GCACGTGAACAATGCCACCAATAAGGCCACATACAAGCCAAACACCTGGTGCATC
AGATGTCTGTGGTCTACAAAGCCCGTGGAGACCAGCAATTCCTTTGATGTGCTGAA
GAGCGAGGATGCCCAGGGCATGGACAACCTGGCCTGCGAGGACCTGAAGCCCGT
GAGCGAGGAGGTGGTGGAGAATCCTACCATCCAGAAGGATGTGCTGGAGTGTAAC
GTGAAGACAACCGAGGTGGTGGGCGACATCATCCTGAAGCCTGCCAACAATTCCC
TGAAGATCACAGAGGAAGTGGGCCACACCGATCTGATGGCCGCCTACGTGGACAA
TTCTAGCCTGACCATCAAGAAGCCAAACGAGCTGAGCAGGGTGCTGGGCCTGAAG
ACCCTGGCCACACACGGCCTGGCCGCAGTGAATTCCGTGCCATGGGACACCATCG
CCAATTATGCCAAGCCCTTCCTGAACAAGGTGGTGAGCACAACCACAAACATCGT
GACACGGTGCCTGAACCGGGTGTGCACCAATTACATGCCATATTTCTTTACACTGC
TGCTGCAGCTGTGCACCTTTACAAGGTCCACCAATTCTCGCATCAAGGCCTCCATG
CCCACCACAATCGCCAAGAACACAGTGAAGAGCGTGGGCAAGTTCTGCCTGGAG
GCCTCCTTTAACTACCTGAAGTCCCCCAATTTCTCTAAGCTGATCAACATCATCATC
TGGTTTCTGCTGCTGAGCGTGTGCCTGGGCAGCCTGATCTATTCCACAGCCGCCCT
GGGCGTGCTGATGAGCAACCTGGGCATGCCTTCCTACTGCACCGGCTATCGGGAG
GGCTACCTGAATAGCACCAACGTGACAATCGCCACCTACTGTACAGGCTCTATCCC
ATGCAGCGTGTGCCTGTCCGGCCTGGATTCTCTGGACACCTATCCTTCCCTGGAGA
CCATCCAGATCACAATCTCCTCTTTCAAGTGGGACCTGACCGCCTTTGGCCTGGTG
GCAGAGTGGTTCCTGGCCTATATCCTGTTTACAAGATTCTTTTACGTGCTGGGCCTG
GCCGCCATCATGCAGCTGTTCTTTAGCTACTTCGCCGTGCACTTTATCTCTAATAGC
TGGCTGATGTGGCTGATCATCAACCTGGTGCAGATGGCCCCCATCTCCGCCATGGT
GAGGATGTATATCTTCTTTGCCTCTTTCTACTACGTGTGGAAGAGCTACGTGCACGT
GGTGGACGGCTGCAATAGCTCCACCTGCATGATGTGCTACAAGAGGAACCGCGCC
ACACGCGTGGAGTGTACCACAATCGTGAATGGCGTGCGGAGAAGCTTCTACGTGT
ATGCCAACGGCGGCAAGGGCTTTTGCAAGCTGCACAACTGGAATTGCGTGAACTG
TGATACATTCTGTGCCGGCAGCACCTTTATCTCCGATGAGGTGGCAAGGGACCTGT
CCCTGCAGTTCAAGAGACCAATCAATCCCACCGATCAGTCTAGCTACATCGTGGAC
TCCGTGACAGTGAAGAACGGCTCTATCCACCTGTATTTCGATAAGGCCGGCCAGAA
GACATACGAGAGGCACTCCCTGTCTCACTTTGTGAATCTGGACAACCTGCGCGCC
AACAATACCAAGGGCAGCCTGCCCATCAACGTGATCGTGTTCGATGGCAAGTCCA
AGTGCGAGGAGTCCTCTGCCAAGAGCGCCTCCGTGTACTATAGCCAGCTGATGTGC
CAGCCTATCCTGCTGCTGGACCAGGCCCTGGTGTCCGATGTGGGCGACTCTGCCGA
GGTGGCAGTGAAGATGTTTGATGCCTACGTGAATACCTTCAGCAGCACCTTCAACG
TGCCAATGGAGAAGCTGAAGACCCTGGTGGCAACAGCAGAGGCAGAGCTGGCCA
AGAACGTGTCCCTGGACAATGTGCTGTCTACCTTCATCAGCGCCGCCCGCCAGGG
CTTTGTGGATTCTGACGTGGAGACAAAGGATGTGGTGGAGTGCCTGAAGCTGAGC
CACCAGTCCGATATCGAGGTGACCGGCGACAGCTGTAACAATTATATGCTGACCTA
CAATAAGGTGGAGAACATGACACCCCGGGATCTGGGCGCCTGCATCGACTGTTCT
GCCAGACACATCAATGCCCAGGTGGCCAAGAGCCACAATATCGCCCTGATCTGGA
ACGTGAAGGACTTCATGTCTCTGAGCGAGCAGCTGAGGAAGCAGATCCGCTCCGC
CGCCAAGAAGAACAATCTGCCCTTCAAGCTGACCTGCGCCACCACAAGGCAGGTG
GTGAACGTGGTCACCACAAAGATCGCCCTGAAGGGCGGC.
The nucleotide sequence of nsp 4 was shown in
SEQ ID NO: 4:
(SEQ ID NO: 4)
AAGATCGTGAACAATTGGCTGAAGCAGCTGATCAAGGTGACCCTGGTGTTCC
TGTTTGTGGCCGCCATCTTCTACCTGATCACCCCCGTGCACGTGATGTCTAAGCAC
ACAGATTTTTCTAGCGAGATCATCGGCTATAAGGCCATCGACGGAGGAGTGACCAG
GGATATCGCCAGCACCGACACATGCTTCGCCAATAAGCACGCCGATTTCGACACCT
GGTTTAGCCAGAGGGGCGGCTCCTACACAAACGACAAGGCCTGTCCACTGATCGC
AGCCGTGATCACCAGGGAAGTGGGATTCGTGGTGCCTGGACTGCCAGGAACAATC
CTGAGGACCACAAATGGCGACTTCCTGCACTTTCTGCCTCGCGTGTTTTCCGCCGT
GGGCAACATCTGCTATACCCCATCTAAGCTGATCGAGTACACCGATTTCGCCACATC
CGCCTGCGTGCTGGCCGCAGAGTGTACCATCTTTAAGGATGCCTCTGGCAAGCCCG
TGCCTTACTGTTATGACACAAATGTGCTGGAGGGCTCTGTGGCCTATGAGAGCCTG
CGGCCAGATACCAGATACGTGCTGATGGACGGCAGCATCATCCAGTTCCCCAACAC
ATATCTGGAGGGCTCTGTGCGGGTGGTGACCACATTTGACAGCGAGTACTGCCGGC
ACGGCACCTGTGAGAGATCTGAGGCCGGCGTGTGCGTGTCCACATCTGGCAGGTG
GGTGCTGAACAATGATTACTATCGCAGCCTGCCTGGCGTGTTCTGTGGCGTGGACG
CCGTGAATCTGCTGACCAACATGTTTACACCTCTGATCCAGCCAATCGGCGCCCTG
GATATCAGCGCCTCCATCGTGGCAGGAGGAATCGTGGCAATCGTGGTGACATGCCT
GGCCTACTATTTCATGCGGTTCCGGAGGGCCTTCGGCGAGTACTCTCACGTGGTGG
CCTTTAATACCCTGCTGTTCCTGATGAGCTTCACCGTGCTGTGCCTGACCCCCGTGT
ATAGCTTCCTGCCTGGCGTGTACTCCGTGATCTACCTGTATCTGACCTTCTACCTGA
CAAACGACGTGAGCTTTCTGGCCCACATCCAGTGGATGGTCATGTTCACCCCCCTG
GTGCCTTTTTGGATCACAATCGCCTATATCATCTGCATCTCCACCAAGCACTTCTATT
GGTTCTTTTCTAATTACCTGAAGCGGAGAGTGGTGTTTAACGGCGTGTCTTTCAGC
ACCTTTGAGGAGGCCGCCCTGTGCACATTCCTGCTGAACAAGGAGATGTACCTGA
AGCTGCGGTCCGACGTGCTGCTGCCACTGACCCAGTACAATAGATATCTGGCCCTG
TATAACAAGTACAAGTATTTCTCTGGCGCCATGGATACCACAAGCTACAGAGAGGC
AGCATGCTGTCACCTGGCAAAGGCCCTGAATGATTTTTCCAACTCTGGCAGCGACG
TGCTGTACCAGCCCCCTCAGACCTCTATCACAAGCGCCGTGCTGCAGTAA.
The nucleotide sequence of nsp 5 was shown in
SEQ ID NO: 5:
(SEQ ID NO: 5)
AGTGGTTTTAGAAAAATGGCATTCCCATCTGGTAAAGTTGAGGGTTGTATGGT
ACAAGTAACTTGTGGTACAACTACACTTAACGGTCTTTGGCTTGATGACGTAGTTT
ACTGTCCAAGACATGTGATCTGCACCTCTGAAGACATGCTTAACCCTAATTATGAA
GATTTACTCATTCGTAAGTCTAATCATAATTTCTTGGTACAGGCTGGTAATGTTCAAC
TCAGGGTTATTGGACATTCTATGCAAAATTGTGTACTTAAGCTTAAGGTTGATACAG
CCAATCCTAAGACACCTAAGTATAAGTTTGTTCGCATTCAACCAGGACAGACTTTT
TCAGTGTTAGCTTGTTACAATGGTTCACCATCTGGTGTTTACCAATGTGCTATGAGG
CCCAATTTCACTATTAAGGGTTCATTCCTTAATGGTTCATGTGGTAGTGTTGGTTTTA
ACATAGATTATGACTGTGTCTCTTTTTGTTACATGCACCATATGGAATTACCAACTGG
AGTTCATGCTGGCACAGACTTAGAAGGTAACTTTTATGGACCTTTTGTTGACAGGC
AAACAGCACAAGCAGCTGGTACGGACACAACTATTACAGTTAATGTTTTAGCTTGG
TTGTACGCTGCTGTTATAAATGGAGACAGGTGGTTTCTCAATCGATTTACCACAACT
CTTAATGACTTTAACCTTGTGGCTATGAAGTACAATTATGAACCTCTAACACAAGAC
CATGTTGACATACTAGGACCTCTTTCTGCTCAAACTGGAATTGCCGTTTTAGATATG
TGTGCTTCATTAAAAGAATTACTGCAAAATGGTATGAATGGACGTACCATATTGGGT
AGTGCTTTATTAGAAGATGAATTTACACCTTTTGATGTTGTTAGACAATGCTCAGGT
GTTACTTTCCAA.
The nucleotide sequence of nsp 6 was shown in
SEQ ID NO: 6:
(SEQ ID NO: 6)
AGTGCAGTGAAAAGAACAATCAAGGGTACACACCACTGGTTGTTACTCACAA
TTTTGACTTCACTTTTAGTTTTAGTCCAGAGTACTCAATGGTCTTTGTTCTTTTTTTT
GTATGAAAATGCCTTTTTACCTTTTGCTATGGGTATTATTGCTATGTCTGCTTTTGCA
ATGATGTTTGTCAAACATAAGCATGCATTTCTCTGTTTGTTTTTGTTACCTTCTCTTG
CCACTGTAGCTTATTTTAATATGGTCTATATGCCTGCTAGTTGGGTGATGCGTATTAT
GACATGGTTGGATATGGTTGATACTAGTTTGTCTGGTTTTAAGCTAAAAGACTGTGT
TATGTATGCATCAGCTGTAGTGTTACTAATCCTTATGACAGCAAGAACTGTGTATGA
TGATGGTGCTAGGAGAGTGTGGACACTTATGAATGTCTTGACACTCGTTTATAAAG
TTTATTATGGTAATGCTTTAGATCAAGCCATTTCCATGTGGGCTCTTATAATCTCTGTT
ACTTCTAACTACTCAGGTGTAGTTACAACTGTCATGTTTTTGGCCAGAGGTATTGTT
TTTATGTGTGTTGAGTATTGCCCTATTTTCTTCATAACTGGTAATACACTTCAGTGTA
TAATGCTAGTTTATTGTTTCTTAGGCTATTTTTGTACTTGTTACTTTGGCCTCTTTTGT
TTACTCAACCGCTACTTTAGACTGACTCTTGGTGTTTATGATTACTTAGTTTCTACAC
AGGAGTTTAGATATATGAATTCACAGGGACTACTCCCACCCAAGAATAGCATAGAT
GCCTTCAAACTCAACATTAAATTGTTGGGTGTTGGTGGCAAACCTTGTATCAAAGT
AGCCACTGTACAG.
The nucleotide sequence of nsp 7 was shown in
SEQ ID NO: 7:
(SEQ ID NO: 7)
TCTAAAATGTCAGATGTAAAGTGCACATCAGTAGTCTTACTCTCAGTTTTGCAA
CAACTCAGAGTAGAATCATCATCTAAATTGTGGGCTCAATGTGTCCAGTTACACAA
TGACATTCTCTTAGCTAAAGATACTACTGAAGCCTTTGAAAAAATGGTTTCACTACT
TTCTGTTTTGCTTTCCATGCAGGGTGCTGTAGACATAAACAAGCTTTGTGAAGAAA
TGCTGGACAACAGGGCAACCTTACAA.
The nucleotide sequence of nsp 8 was shown in
SEQ ID NO: 8:
(SEQ ID NO: 8)
GCTATAGCCTCAGAGTTTAGTTCCCTTCCATCATATGCAGCTTTTGCTACTGCTC
AAGAAGCTTATGAGCAGGCTGTTGCTAATGGTGATTCTGAAGTTGTTCTTAAAAAG
TTGAAGAAGTCTTTGAATGTGGCTAAATCTGAATTTGACCGTGATGCAGCCATGCA
ACGTAAGTTGGAAAAGATGGCTGATCAAGCTATGACCCAAATGTATAAACAGGCTA
GATCTGAGGACAAGAGGGCAAAAGTTACTAGTGCTATGCAGACAATGCTTTTCACT
ATGCTTAGAAAGTTGGATAATGATGCACTCAACAACATTATCAACAATGCAAGAGA
TGGTTGTGTTCCCTTGAACATAATACCTCTTACAACAGCAGCCAAACTAATGGTTGT
CATACCAGACTATAACACATATAAAAATACGTGTGATGGTACAACATTTACTTATGC
ATCAGCATTGTGGGAAATCCAACAGGTTGTAGATGCAGATAGTAAAATTGTTCAAC
TTAGTGAAATTAGTATGGACAATTCACCTAATTTAGCATGGCCTCTTATTGTAACAG
CTTTAAGGGCCAATTCTGCTGTCAAATTACAG.
The nucleotide sequence of nsp 9 was shown in
SEQ ID NO: 9:
(SEQ ID NO: 9)
AATAATGAGCTTAGTCCTGTTGCACTACGACAGATGTCTTGTGCTGCCGGTACT
ACACAAACTGCTTGCACTGATGACAATGCGTTAGCTTACTACAACACAACAAAGG
GAGGTAGGTTTGTACTTGCACTGTTATCCGATTTACAGGATTTGAAATGGGCTAGAT
TCCCTAAGAGTGATGGAACTGGTACTATCTATACAGAACTGGAACCACCTTGTAGG
TTTGTTACAGACACACCTAAAGGTCCTAAAGTGAAGTATTTATACTTTATTAAAGGA
TTAAACAACCTAAATAGAGGTATGGTACTTGGTAGTTTAGCTGCCACAGTACGTCTA
CAA.
The nucleotide sequence of nsp 10 was shown in
SEQ ID NO: 10:
(SEQ ID NO: 10)
GCTGGTAATGCAACAGAAGTGCCTGCCAATTCAACTGTATTATCTTTCTGTGCT
TTTGCTGTAGATGCTGCTAAAGCTTACAAAGATTATCTAGCTAGTGGGGGACAACC
AATCACTAATTGTGTTAAGATGTTGTGTACACACACTGGTACTGGTCAGGCAATAA
CAGTTACACCGGAAGCCAATATGGATCAAGAATCCTTTGGTGGTGCATCGTGTTGT
CTGTACTGCCGTTGCCACATAGATCATCCAAATCCTAAAGGATTTTGTGACTTAAAA
GGTAAGTATGTACAAATACCTACAACTTGTGCTAATGACCCTGTGGGTTTTACACTT
AAAAACACAGTCTGTACCGTCTGCGGTATGTGGAAAGGTTATGGCTGTAGTTGTGA
TCAACTCCGCGAACCCATGCTTCAG.
The nucleotide sequence of nsp 11 was shown in
SEQ ID NO: 11:
(SEQ ID NO: 11)
TCAGCTGATGCACAATCGTTTTTAAACGGGTTTGCGGTG.
The nucleotide sequence of nsp 12 was shown in
SEQ ID NO: 12:
(SEQ ID NO: 12)
ATGTCAGCAGATGCACAATCATTTCTTAACAGAGTGTGCGGAGTGTCAGCAGC
AAGACTTACACCTTGCGGAACAGGAACATCAACAGATGTAGTTTATAGGGCCTTCG
ATATCTACAACGATAAAGTGGCAGGATTTGCAAAGTTCTTAAAGACCAATTGCTGC
AGATTTCAAGAGAAGGACGAGGATGATAACCTTATCGATTCATACTTTGTGGTGAA
GAGGCATACATTCAGCAATTACCAACACGAAGAAACAATCTACAACCTTCTTAAAG
ATTGCCCTGCAGTGGCAAAGCATGACTTCTTCAAGTTCAGAATCGATGGAGATATG
GTGCCTCACATCTCAAGACAAAGACTTACAAAGTATACGATGGCAGATCTCGTTTA
TGCGTTGCGCCATTTCGACGAGGGTAATTGTGACACCCTGAAGGAGATCCTGGTCA
CGTATAATTGCTGCGATGATGATTACTTTAACAAGAAGGACTGGTATGATTTCGTAG
AGAATCCTGACATTCTTAGAGTGTACGCAAACCTTGGAGAAAGAGTGAGACAAGC
ACTCCTAAAGACAGTTCAATTCTGCGACGCAATGAGAAACGCAGGAATCGTGGGA
GTGCTTACACTTGATAACCAAGATCTTAACGGAAACTGGTATGACTTTGGCGACTT
TATACAGACAACACCTGGATCAGGAGTGCCTGTGGTGGATTCATATTATAGCCTGCT
GATGCCTATCCTTACACTTACAAGAGCACTTACAGCAGAATCACATGTGGATACCG
ACTTGACCAAACCCTATATTAAATGGGATCTGCTGAAATATGACTTTACAGAAGAA
CGACTTAAACTCTTCGACAGATACTTTAAATACTGGGATCAAACATACCACCCTAA
CTGCGTGAACTGCCTTGATGATAGATGCATCCTTCACTGCGCAAACTTTAACGTGC
TGTTCTCGACCGTGTTTCCTCCTACATCATTTGGACCTCTTGTGAGAAAGATCTTTG
TGGACGGAGTACCTTTCGTCGTATCAACAGGATACCACTTTAGAGAACTTGGAGTA
GTGCATAATCAAGATGTGAACCTACATTCTAGCCGATTATCATTTAAAGAACTTCTG
GTTTATGCCGCGGACCCTGCAATGCACGCAGCAAGTGGCAATTTATTACTTGACAA
ACGGACAACCTGTTTCTCGGTTGCCGCACTTACAAACAATGTAGCTTTCCAGACCG
TAAAGCCAGGGAATTTCAACAAAGATTTCTATGACTTCGCCGTATCAAAGGGATTC
TTCAAGGAGGGATCATCAGTGGAACTTAAACACTTCTTCTTCGCCCAGGATGGAA
ACGCAGCAATCTCAGATTACGATTACTACAGATACAACCTTCCTACAATGTGCGATA
TCAGACAACTTCTCTTCGTAGTTGAAGTGGTGGATAAATACTTTGATTGCTACGATG
GAGGATGCATCAACGCAAACCAAGTGATCGTGAACAACTTGGATAAATCCGCTGG
ATTCCCGTTTAATAAGTGGGGTAAAGCCCGCCTTTACTACGATTCAATGTCATACGA
AGATCAAGATGCATTATTCGCTTATACAAAGAGGAATGTGATCCCTACAATCACACA
AATGAACCTTAAATACGCAATCTCAGCAAAGAATCGAGCAAGAACAGTGGCAGGA
GTGTCAATCTGCTCAACAATGACAAACAGACAATTTCACCAGAAGCTCCTGAAAT
CAATCGCAGCAACAAGAGGAGCAACAGTGGTGATCGGAACATCAAAGTTCTATGG
AGGTTGGCACAACATGCTCAAGACCGTGTATAGCGATGTTGAGAATCCGCATCTCA
TGGGATGGGATTACCCTAAATGCGATAGAGCTATGCCCAATATGCTGAGAATCATGG
CATCACTTGTGCTTGCAAGAAAGCATACCACATGCTGCTCACTTTCACACAGATTC
TATCGACTTGCAAACGAATGCGCACAGGTCCTCTCCGAGATGGTGATGTGCGGCG
GGAGCTTGTATGTGAAACCAGGTGGAACATCATCAGGAGATGCAACAACAGCATA
CGCAAACTCAGTGTTTAACATCTGCCAAGCAGTGACAGCTAATGTAAACGCTCTCT
TGAGCACTGACGGAAACAAGATAGCCGATAAATACGTGCGTAATCTGCAGCATCGA
CTTTACGAATGCCTTTACAGAAACAGAGATGTAGACACGGACTTTGTAAATGAATT
CTATGCTTACCTTAGAAAGCATTTCTCCATGATGATACTGAGTGACGATGCTGTTGT
ATGTTTCAACTCAACATACGCATCACAAGGACTTGTGGCATCAATCAAGAATTTCA
AATCAGTGCTTTACTACCAGAATAATGTGTTTATGTCAGAAGCAAAGTGTTGGACA
GAAACTGACCTCACTAAGGGCCCTCACGAGTTCTGTAGCCAACACACAATGCTTG
TGAAACAAGGAGATGACTATGTTTATCTCCCATACCCTGATCCTTCAAGAATCTTGG
GTGCAGGGTGTTTCGTGGATGATATCGTGAAGACTGACGGAACACTTATGATCGAA
AGATTTGTGTCACTTGCAATCGATGCATACCCTCTTACAAAGCATCCGAACCAAGA
ATACGCAGATGTGTTTCACCTTTACCTTCAATACATCAGAAAGTTGCATGATGAACT
TACAGGACACATGCTTGATATGTACTCAGTGATGCTTACAAACGATAACACATCAA
GATACTGGGAACCTGAATTCTATGAGGCAATGTACACACCTCACACAGTGCTTCAA.
The nucleotide sequence of nsp 13 was shown in
SEQ ID NO: 13:
(SEQ ID NO: 13)
GCAGTGGGAGCATGCGTGCTTTGCAACTCACAAACATCACTTAGATGCGGAG
CATGCATCAGAAGACCTTTCCTGTGTTGCAAATGCTGCTACGATCACGTGATCTCA
ACATCACACAAACTTGTGCTTTCAGTGAACCCTTACGTGTGCAACGCACCAGGCT
GTGACGTAACTGACGTTACGCAGCTCTATCTTGGAGGAATGTCATACTACTGCAAA
TCACACAAACCTCCTATCTCATTTCCTCTTTGCGCAAACGGACAAGTGTTTGGACT
TTACAAGAATACTTGCGTGGGATCAGATAACGTGACAGATTTCAATGCTATCGCAA
CATGCGATTGGACAAACGCAGGAGATTACATCCTTGCAAACACATGCACAGAGCG
TCTGAAGTTGTTTGCGGCCGAAACACTTAAAGCAACAGAAGAAACATTTAAACTT
TCATACGGAATCGCAACAGTGAGAGAGGTCCTATCGGACAGGGAACTCCACCTTT
CATGGGAAGTGGGCAAACCACGCCCGCCGCTTAACAGAAACTACGTGTTTACAGG
ATACAGAGTGACAAAGAATTCTAAGGTACAGATCGGAGAATACACATTTGAGAAG
GGCGACTACGGAGACGCCGTGGTGTACAGAGGGACGACTACGTATAAACTTAACG
TGGGAGATTACTTTGTGCTTACATCACACACAGTGATGCCTCTTTCAGCACCTACA
CTTGTGCCTCAAGAGCATTATGTCCGAATAACGGGTCTCTATCCGACACTTAACATC
TCAGATGAATTCTCGAGTAACGTGGCAAACTACCAGAAAGTGGGTATGCAGAAAT
ACTCCACCTTACAGGGACCTCCTGGTACAGGAAAGTCTCATTTCGCGATAGGTCTA
GCTCTCTATTACCCTTCAGCAAGAATCGTGTACACAGCATGCTCACACGCAGCAGT
GGATGCACTTTGCGAGAAGGCGCTGAAATACCTTCCTATCGATAAATGCTCAAGAA
TCATCCCTGCAAGAGCAAGAGTGGAATGCTTTGATAAATTTAAAGTGAACTCAACA
CTTGAACAATACGTGTTCTGTACTGTAAATGCTCTGCCTGAAACTACCGCGGATATC
GTGGTGTTCGACGAGATATCCATGGCAACAAACTACGACCTATCGGTCGTAAACGC
GCGGCTAAGAGCAAAGCATTATGTGTACATCGGAGATCCTGCACAACTTCCTGCAC
CTAGAACATTACTAACTAAAGGGACGCTCGAACCTGAATACTTTAACAGTGTTTGT
CGCCTAATGAAGACGATCGGGCCGGACATGTTTCTTGGAACATGCAGAAGATGCC
CTGCAGAAATCGTGGATACAGTGTCAGCACTTGTGTACGATAACAAACTTAAAGCA
CACAAAGACAAGTCGGCTCAGTGTTTCAAGATGTTTTACAAAGGAGTGATCACAC
ACGATGTGTCATCAGCAATCAACAGACCTCAAATCGGAGTGGTGAGAGAATTTCTT
ACAAGAAACCCTGCATGGAGAAAGGCGGTCTTCATAAGTCCTTACAACTCACAGA
ATGCCGTGGCATCAAAGATACTCGGGCTTCCTACACAAACAGTGGATTCATCACAA
GGATCAGAATACGATTACGTGATCTTTACACAAACAACAGAAACAGCACACTCATG
CAACGTGAACAGATTTAACGTGGCAATCACAAGAGCAAAGGTAGGGATCCTCTGT
ATCATGTCAGATAGAGATCTTTACGATAAACTTCAATTTACATCACTTGAAATCCCT
AGAAGAAACGTGGCGACTCTGCAG.
The nucleotide sequence of nsp 14 was shown in
SEQ ID NO: 14:
(SEQ ID NO: 14)
GCTGAGAACGTGACAGGATTGTTCAAGGACTGCTCAAAGGTAATTACGGGTT
TACATCCGACACAAGCACCTACACACCTTTCAGTGGATACAAAGTTCAAGACTGA
AGGACTTTGCGTGGATATCCCTGGAATCCCTAAAGATATGACATACAGAAGACTTAT
CTCAATGATGGGATTTAAGATGAATTACCAAGTGAACGGATACCCTAACATGTTTAT
CACAAGAGAAGAAGCAATCAGACACGTGAGAGCATGGATAGGCTTCGACGTCGA
GGGATGCCACGCAACAAGAGAAGCAGTGGGAACAAACCTTCCTCTTCAACTTGG
ATTCTCCACTGGAGTGAACCTTGTGGCAGTGCCTACAGGATACGTGGATACACCTA
ACAACACAGATTTCTCGCGAGTGTCAGCAAAGCCACCACCTGGAGATCAATTTAA
ACACCTTATCCCTCTTATGTACAAAGGACTTCCTTGGAACGTGGTGAGAATCAAGA
TAGTCCAAATGCTATCCGATACCTTAAAGAATCTTAGTGACCGTGTCGTATTTGTGC
TTTGGGCACACGGATTTGAACTTACATCAATGAAATACTTTGTGAAGATCGGTCCC
GAGCGTACATGCTGCCTTTGCGATAGAAGAGCTACGTGTTTCAGTACCGCTTCAGA
TACATACGCATGCTGGCACCACTCAATAGGCTTCGATTACGTTTATAATCCGTTCAT
GATAGATGTGCAACAATGGGGATTCACGGGCAATCTGCAGAGCAACCACGATCTTT
ACTGCCAAGTGCACGGAAACGCACACGTGGCATCATGCGATGCAATCATGACAAG
ATGCCTTGCAGTGCACGAATGCTTTGTGAAGCGGGTCGATTGGACAATCGAATACC
CTATCATCGGAGATGAACTTAAGATAAATGCAGCATGCAGAAAGGTCCAGCACATG
GTGGTGAAAGCAGCACTTCTTGCAGATAAATTTCCTGTGCTTCACGATATCGGAAA
CCCTAAAGCAATCAAATGCGTGCCTCAAGCAGATGTGGAATGGAAATTCTATGACG
CACAACCTTGCTCAGATAAAGCATACAAGATAGAGGAACTATTCTATAGTTACGCA
ACACACTCAGATAAATTTACAGATGGAGTGTGCCTGTTCTGGAATTGCAACGTGGA
TAGATACCCTGCAAACTCAATCGTGTGCAGATTTGATACAAGAGTGCTTTCAAACC
TTAACCTTCCAGGTTGTGACGGCGGCAGTCTATATGTTAATAAGCACGCATTTCACA
CACCTGCATTCGATAAGTCCGCATTCGTCAATTTAAAGCAGCTACCTTTCTTCTATT
ATTCAGATTCACCTTGCGAATCACACGGAAAGCAGGTTGTCAGTGACATCGATTAC
GTGCCTCTTAAATCAGCAACATGTATTACCAGGTGTAATCTTGGAGGAGCCGTCTG
TCGACATCATGCAAACGAATACAGACTTTACCTTGATGCATACAACATGATGATCTC
CGCCGGGTTCTCCCTATGGGTGTACAAACAATTTGATACATACAACCTTTGGAACA
CATTTACAAGACTTCAA.
The nucleotide sequence of nsp 15 was shown in
SEQ ID NO: 15:
(SEQ ID NO: 15)
TCACTTGAGAACGTTGCGTTCAATGTAGTCAATAAGGGACACTTCGACGGTCA
ACAGGGTGAGGTTCCTGTGTCAATCATCAACAATACCGTTTATACTAAAGTTGACG
GCGTGGATGTGGAACTCTTCGAGAATAAGACTACGCTTCCTGTGAATGTTGCCTTC
GAGTTGTGGGCAAAGCGCAATATCAAACCTGTGCCTGAAGTGAAGATACTCAATA
ACCTTGGAGTGGATATCGCAGCAAACACAGTGATCTGGGATTACAAGAGGGACGC
ACCTGCACACATCTCAACAATCGGAGTGTGCTCAATGACAGATATCGCAAAGAAG
CCGACTGAAACAATCTGCGCACCTCTTACTGTATTCTTCGACGGAAGAGTGGATGG
ACAAGTGGATTTATTCCGAAATGCAAGAAACGGAGTGCTTATCACAGAAGGATCA
GTGAAAGGACTTCAACCTTCAGTGGGACCTAAACAAGCATCACTTAACGGAGTGA
CTCTGATAGGCGAGGCCGTGAAGACTCAGTTTAACTACTACAAGAAAGTAGACGG
TGTCGTCCAGCAGCTGCCCGAGACCTATTTCACACAATCACGGAATCTGCAGGAGT
TCAAACCTAGATCACAAATGGAAATCGATTTCCTGGAGCTTGCAATGGATGAATTT
ATCGAAAGATACAAACTTGAAGGATACGCATTTGAACACATCGTGTACGGAGATTT
CAGTCATTCACAACTTGGAGGACTTCACCTTCTTATTGGCCTAGCCAAACGTTTCA
AAGAATCACCTTTCGAGCTCGAAGATTTCATTCCAATGGATTCAACAGTGAAGAAT
TATTTCATTACTGACGCCCAGACGGGATCATCAAAGTGTGTATGCTCAGTGATCGAT
CTACTACTAGACGATTTCGTTGAAATTATTAAATCACAAGACTTGAGTGTAGTTAGT
AAGGTTGTGAAGGTCACAATCGATTACACAGAAATCTCATTTATGCTTTGGTGCAA
AGATGGACACGTGGAAACATTCTATCCCAAACTTCAA.
The nucleotide sequence of nsp 16 was shown in
SEQ ID NO: 16:
(SEQ ID NO: 16)
TCATCACAAGCATGGCAACCTGGAGTGGCCATGCCGAATTTGTATAAGATGCA
GAGAATGCTTCTTGAGAAGTGTGACCTTCAGAATTATGGAGATTCAGCAACACTTC
CTAAAGGAATCATGATGAACGTGGCAAAGTATACTCAACTTTGCCAATACCTTAAC
ACACTTACACTTGCAGTGCCTTACAACATGAGAGTGATCCACTTCGGTGCAGGGTC
GGACAAAGGAGTGGCACCTGGTACTGCTGTCCTTAGACAATGGCTTCCTACAGGA
ACACTTCTTGTGGATTCAGATCTTAACGATTTCGTCTCCGATGCAGATTCAACCCTC
ATTGGTGACTGTGCAACAGTGCACACAGCAAACAAGTGGGACTTAATAATATCAG
ATATGTACGATCCTAAGACTAAGAATGTAACGAAAGAGAATGACTCAAAGGAAGG
TTTCTTCACCTATATCTGCGGATTTATCCAACAGAAGTTAGCTCTTGGAGGATCAGT
GGCAATCAAGATTACGGAACACTCATGGAACGCAGATCTTTACAAACTTATGGGAC
ACTTTGCATGGTGGACCGCGTTCGTTACAAACGTAAACGCGTCGTCCTCAGAAGC
ATTTCTTATCGGATGCAACTACCTTGGGAAACCAAGAGAGCAGATCGATGGATACG
TGATGCACGCAAACTACATCTTCTGGAGGAACACAAACCCTATCCAACTTTCATCA
TACTCACTCTTCGACATGTCAAAGTTCCCGCTTAAACTTAGAGGGACTGCCGTAAT
GTCGCTTAAAGAAGGACAAATCAACGATATGATACTCAGCCTCCTAAGTAAAGGGA
GGCTTATCATCAGAGAGAATAATAGAGTGGTGATCTCATCAGATGTGCTTGTGAAC
AACTAA.

In this example, the ps2AN molecule was derived from NSP1-NSP4 sequences on N′ end of ORF1a of SARS-COV-2, and the sequences had been codon-optimized for human; the ps2AN molecule was derived from NSP5-NSP11 sequences on C′ end of ORF1a of SARS-CoV-2, and the sequences had been codon-optimized for human; and the ps2B molecule was derived from NSP12-NSP16 sequences on C′ end of ORF1ab of SARS-COV-2, and the sequences had been codon-optimized for human.
ps2AN comprised nsps 1-4, totaling 10429 bp;
ps2AC comprised nsp5-nsp11, totaling 4012 bp; and
ps2B comprised nsp12-nsp16, totaling 8641 bp.

The nucleotide sequence of ps2AN was shown in SEQ ID NO: 17:
SEQ ID NO: 17
GCTAGCGAGGGCCCGGAAACCTGGCCCTGTCTTCTTGACGAGCATTCCTAGG
GGTCTTTCCCCTCTCGCCAAAGGAATGCAAGGTCTGTTGAATGTCGTGAAGGAAG
CAGTTCCTCTGGAAGCTTCTTGAAGACAAACAACGTCTGTAGCGACCCTTTGCAG
GCAGCGGAACCCCCCACCTGGCGACAGGTGCCTCTGCGGCCAAAAGCCACGTGT
ATAAGATACACCTGCAAAGGCGGCACAACCCCAGTGCCACGTTGTGAGTTGGATA
GTTGTGGAAAGAGTCAAATGGCTCTCCTCAAGCGTATTCAACAAGGGGCTGAAGG
ATGCCCAGAAGGTACCCCATTGTATGGGATCTGATCTGGGGCCTCGGTGCACATGC
TTTACATGTGTTTAGTCGAGGTTAAAAAAACGTCTAGGCCCCCCGAACCACGGGG
ACGTGGTTTTCCTTTGAAAAACACGATGATAAATGGAGTCCCTGGTGCCCGGCTTC
AACGAGAAGACCCACGTGCAGCTGTCTCTGCCTGTGCTGCAGGTGAGGGATGTGC
TGGTGCGCGGCTTTGGCGACTCCGTCGAGGAGGTGCTGTCTGAGGCCAGGCAGCA
CCTGAAGGACGGAACCTGCGGACTGGTGGAGGTGGAGAAGGGCGTGCTGCCACA
GCTGGAGCAGCCTTACGTGTTCATCAAGAGGTCCGATGCAAGGACAGCACCACAC
GGACACGTGATGGTGGAGCTGGTGGCCGAGCTGGAGGGCATCCAGTATGGCCGCT
CTGGAGAGACCCTGGGCGTGCTGGTGCCACACGTGGGAGAGATCCCAGTGGCCTA
TCGGAAGGTGCTGCTGAGAAAGAACGGCAATAAGGGAGCAGGAGGACACTCTTA
CGGAGCAGACCTGAAGAGCTTCGATCTGGGCGACGAGCTGGGCACCGATCCTTAT
GAGGACTTTCAGGAGAACTGGAATACAAAGCACAGCTCCGGCGTGACCCGGGAG
CTGATGAGAGAGCTGAACGGCGGCGCCTACACCAGATATGTGGATAACAATTTCTG
CGGACCAGACGGATACCCCCTGGAGTGTATCAAGGATCTGCTGGCCAGAGCAGGC
AAGGCCTCCTGCACCCTGTCTGAGCAGCTGGACTTCATCGACACAAAGCGGGGCG
TGTATTGCTGTAGAGAGCACGAGCACGAGATCGCCTGGTATACCGAGCGGTCCGA
GAAGTCTTACGAGCTGCAGACACCATTCGAGATCAAGCTGGCCAAGAAGTTCGAC
ACCTTCAACGGCGAGTGTCCAAACTTCGTGTTTCCCCTGAATAGCATCATCAAGAC
CATCCAGCCCAGAGTGGAGAAGAAGAAGCTGGATGGCTTTATGGGCAGGATCCGC
AGCGTGTACCCTGTGGCCTCCCCAAACGAGTGCAATCAGATGTGCCTGTCCACACT
GATGAAGTGCGATCACTGTGGCGAGACCTCTTGGCAGACAGGCGACTTCGTGAAG
GCCACCTGCGAGTTTTGTGGCACCGAGAACCTGACAAAGGAGGGCGCCACCACAT
GCGGCTATCTGCCTCAGAATGCCGTGGTGAAGATCTACTGCCCAGCCTGTCACAAC
TCCGAAGTGGGACCAGAGCACTCTCTGGCCGAGTACCACAATGAGTCCGGCCTGA
AGACAATCCTGAGGAAGGGAGGAAGGACCATCGCCTTCGGCGGATGCGTGTTTTC
TTATGTGGGCTGCCACAACAAGTGTGCATACTGGGTGCCAAGGGCCAGCGCCAAT
ATCGGCTGTAACCACACCGGAGTGGTGGGAGAGGGATCCGAGGGCCTGAACGATA
ATCTGCTGGAGATCCTGCAGAAGGAGAAGGTGAACATCAATATCGTGGGCGACTT
CAAGCTGAACGAGGAGATCGCCATCATCCTGGCCTCCTTCTCTGCCAGCACATCCG
CCTTTGTGGAGACCGTGAAGGGCCTGGACTACAAGGCCTTCAAGCAGATCGTGGA
GAGCTGCGGCAACTTCAAGGTGACCAAGGGCAAGGCCAAGAAGGGCGCCTGGAA
CATCGGCGAGCAGAAGAGCATCCTGTCCCCTCTGTATGCCTTCGCCAGCGAGGCA
GCAAGGGTGGTGAGATCTATCTTTAGCCGGACCCTGGAGACAGCCCAGAATTCCG
TGAGAGTGCTGCAGAAGGCCGCCATCACCATCCTGGATGGCATCTCCCAGTACTCT
CTGAGGCTGATCGATGCCATGATGTTCACCTCCGACCTGGCCACAAACAATCTGGT
GGTCATGGCCTACATCACCGGCGGCGTGGTGCAGCTGACCTCTCAGTGGCTGACA
AACATCTTTGGCACCGTGTATGAGAAGCTGAAGCCAGTGCTGGATTGGCTGGAGG
AGAAGTTCAAGGAGGGCGTGGAGTTTCTGCGCGACGGCTGGGAGATCGTGAAGT
TCATCAGCACCTGCGCATGTGAGATCGTGGGAGGACAGATCGTGACCTGTGCCAA
GGAGATCAAGGAGTCCGTGCAGACATTCTTTAAGCTGGTGAACAAGTTCCTGGCC
CTGTGCGCCGACTCTATCATCATCGGCGGCGCCAAGCTGAAGGCCCTGAACCTGG
GCGAGACCTTTGTGACACACAGCAAGGGCCTGTACAGGAAGTGCGTGAAGTCCC
GCGAGGAGACCGGACTGCTGATGCCCCTGAAGGCACCTAAGGAGATCATCTTCCT
GGAGGGCGAGACCCTGCCCACAGAGGTGCTGACAGAGGAGGTGGTGCTGAAGAC
CGGCGACCTGCAGCCACTGGAGCAGCCCACCAGCGAGGCAGTGGAGGCACCTCT
GGTGGGCACACCAGTGTGCATCAATGGCCTGATGCTGCTGGAGATCAAGGATACC
GAGAAGTACTGTGCCCTGGCCCCTAACATGATGGTGACAAACAATACCTTCACACT
GAAGGGCGGCGCCCCAACCAAGGTGACATTTGGCGACGATACCGTGATCGAGGTG
CAGGGCTACAAGTCTGTGAATATCACATTCGAGCTGGATGAGAGAATCGACAAGG
TGCTGAACGAGAAGTGCAGCGCCTATACAGTGGAGCTGGGCACCGAGGTGAACG
AGTTTGCCTGCGTGGTGGCCGACGCCGTGATCAAGACCCTGCAGCCAGTGTCCGA
GCTGCTGACACCCCTGGGCATCGATCTGGACGAGTGGTCTATGGCCACCTACTATC
TGTTCGACGAGAGCGGCGAGTTTAAGCTGGCCTCCCACATGTACTGCTCTTTCTAT
CCCCCTGATGAAGACGAGGAGGAGGGCGATTGCGAGGAGGAGGAGTTTGAGCCC
AGCACACAGTACGAGTATGGCACCGAGGACGATTACCAGGGCAAGCCACTGGAGT
TCGGAGCCACCTCCGCCGCCCTGCAGCCAGAGGAGGAGCAGGAGGAGGATTGGC
TGGACGATGACTCCCAGCAGACCGTGGGCCAGCAGGATGGCTCTGAGGACAATCA
GACCACAACCATCCAGACAATCGTGGAGGTGCAGCCTCAGCTGGAGATGGAGCTG
ACCCCAGTGGTGCAGACCATCGAGGTGAACTCTTTCAGCGGCTATCTGAAGCTGA
CAGATAACGTGTACATCAAGAACGCCGACATTGTGGAGGAGGCCAAGAAGGTGAA
GCCTACCGTGGTGGTGAACGCCGCCAACGTGTACCTGAAGCACGGAGGAGGAGT
GGCAGGCGCCCTGAACAAGGCCACCAACAATGCCATGCAGGTGGAGAGCGATGA
CTATATCGCCACAAATGGACCCCTGAAGGTCGGAGGAAGCTGCGTGCTGTCCGGA
CACAACCTGGCCAAGCACTGTCTGCACGTGGTGGGCCCTAACGTGAATAAGGGCG
AGGACATCCAGCTGCTGAAGTCCGCCTACGAGAACTTCAATCAGCACGAGGTGCT
GCTGGCCCCTCTGCTGAGCGCCGGCATCTTTGGCGCCGATCCAATCCACTCCCTGA
GGGTGTGCGTGGACACCGTGCGCACAAACGTGTACCTGGCCGTGTTCGATAAGAA
CCTGTACGACAAGCTGGTGTCTAGCTTTCTGGAGATGAAGAGCGAGAAGCAGGTG
GAGCAGAAGATCGCCGAGATCCCTAAGGAGGAGGTGAAGCCATTCATCACCGAGA
GCAAGCCTTCCGTGGAGCAGAGGAAGCAGGATGACAAGAAGATCAAGGCCTGCG
TGGAGGAGGTGACAACCACACTGGAGGAGACCAAGTTCCTGACAGAGAACCTGC
TGCTGTACATCGATATCAACGGCAATCTGCACCCAGACAGCGCCACACTGGTGTCC
GATATCGACATCACCTTTCTGAAGAAGGATGCCCCATATATCGTGGGCGACGTGGT
GCAGGAGGGCGTGCTGACAGCCGTGGTCATCCCCACCAAGAAGGCCGGCGGCAC
CACAGAGATGCTGGCCAAGGCCCTGCGCAAGGTGCCTACCGACAATTACATCACC
ACATATCCAGGCCAGGGCCTGAACGGCTATACCGTGGAGGAGGCCAAGACCGTGC
TGAAGAAGTGCAAGAGCGCCTTCTACATCCTGCCTTCTATCATCAGCAATGAGAAG
CAGGAGATCCTGGGCACCGTGTCCTGGAACCTGAGGGAGATGCTGGCCCACGCCG
AGGAGACACGCAAGCTGATGCCCGTGTGCGTGGAGACAAAGGCCATCGTGAGCA
CCATCCAGCGGAAGTATAAGGGCATCAAGATCCAGGAGGGAGTGGTGGACTACGG
AGCAAGATTCTACTTTTATACCTCTAAGACCACAGTGGCCAGCCTGATCAACACAC
TGAATGATCTGAACGAGACCCTGGTGACAATGCCCCTGGGCTATGTGACCCACGG
CCTGAATCTGGAGGAGGCCGCCAGGTACATGCGCTCCCTGAAGGTGCCAGCAACC
GTGAGCGTGAGCTCTCCTGACGCCGTGACAGCCTACAACGGCTATCTGACAAGCT
CCTCTAAGACCCCAGAGGAGCACTTCATCGAGACCATCTCTCTGGCCGGCAGCTAT
AAGGATTGGTCCTACTCTGGCCAGTCCACACAGCTGGGCATCGAGTTTCTGAAGA
GGGGCGACAAGAGCGTGTACTATACCAGCAATCCCACCACATTCCACCTGGATGGC
GAAGTGATCACCTTCGACAACCTGAAGACCCTGCTGAGCCTGCGGGAGGTGAGA
ACCATCAAGGTGTTCACCACAGTGGATAACATCAATCTGCACACACAGGTGGTGG
ACATGTCCATGACCTATGGCCAGCAGTTTGGCCCAACATACCTGGATGGCGCCGAC
GTGACCAAGATCAAGCCCCACAATAGCCACGAGGGCAAGACATTCTACGTGCTGC
CTAATGCCACCAACTTTTCCCTGCTGAAGCAGGCAGGCGACGTGGAGGAGAACCC
AGGACCAGATGACACCCTGAGGGTGGAGGCCTTCGAGTACTATCACACCACAGAT
CCTAGCTTTCTGGGCCGCTATATGTCCGCCCTGAATCACACCAAGAAGTGGAAGTA
CCCACAGGTGAACGGCCTGACAAGCATCAAGTGGGCCGACAACAATTGCTACCTG
GCCACCGCCCTGCTGACACTGCAGCAGATCGAGCTGAAGTTCAACCCACCCGCCC
TGCAGGATGCATACTATAGGGCAAGAGCAGGAGAGGCAGCCAATTTTTGCGCCCT
GATCCTGGCCTATTGTAACAAGACCGTGGGAGAGCTGGGCGATGTGCGGGAGACA
ATGAGCTACCTGTTCCAGCACGCCAATCTGGACTCCTGCAAGAGAGTGCTGAACG
TGGTGTGCAAGACATGTGGCCAGCAGCAGACCACACTGAAGGGCGTGGAGGCCG
TGATGTATATGGGCACCCTGAGCTACGAGCAGTTTAAGAAGGGCGTGCAGATCCCC
TGCACATGTGGCAAGCAGGCCACCAAGTACCTGGTGCAGCAGGAGTCCCCTTTCG
TGATGATGTCTGCCCCTCCAGCCCAGTATGAGCTGAAGCACGGCACCTTTACATGC
GCCTCTGAGTACACCGGCAATTATCAGTGTGGCCACTATAAGCACATCACCAGCAA
GGAGACACTGTACTGCATCGATGGCGCCCTGCTGACCAAGAGCTCCGAGTACAAG
GGCCCCATCACAGACGTGTTCTATAAGGAGAATTCTTACACCACAACCATCGCCAC
CAACTTTAGCCTGCTGAAGCAGGCCGGCGATGTGGAGGAGAACCCTGGACCAAA
GCCCGTGACCTATAAGCTGGACGGCGTGGTGTGCACAGAGATCGATCCTAAGCTG
GACAACTACTACAAGAAGGATAACTCTTATTTCACCGAGCAGCCCATCGACCTGGT
GCCTAATCAGCCTTACCCAAACGCCAGCTTCGATAATTTCAAGTTCGTGTGCGACA
ATATCAAGTTTGCCGATGACCTGAACCAGCTGACCGGATACAAGAAGCCAGCCAG
CCGGGAGCTGAAGGTGACATTCTTTCCTGATCTGAACGGCGACGTGGTGGCCATC
GACTACAAGCACTATACACCTTCCTTCAAGAAGGGCGCCAAGCTGCTGCACAAGC
CAATCGTGTGGCACGTGAACAATGCCACCAATAAGGCCACATACAAGCCAAACAC
CTGGTGCATCAGATGTCTGTGGTCTACAAAGCCCGTGGAGACCAGCAATTCCTTTG
ATGTGCTGAAGAGCGAGGATGCCCAGGGCATGGACAACCTGGCCTGCGAGGACCT
GAAGCCCGTGAGCGAGGAGGTGGTGGAGAATCCTACCATCCAGAAGGATGTGCTG
GAGTGTAACGTGAAGACAACCGAGGTGGTGGGCGACATCATCCTGAAGCCTGCCA
ACAATTCCCTGAAGATCACAGAGGAAGTGGGCCACACCGATCTGATGGCCGCCTA
CGTGGACAATTCTAGCCTGACCATCAAGAAGCCAAACGAGCTGAGCAGGGTGCTG
GGCCTGAAGACCCTGGCCACACACGGCCTGGCCGCAGTGAATTCCGTGCCATGGG
ACACCATCGCCAATTATGCCAAGCCCTTCCTGAACAAGGTGGTGAGCACAACCAC
AAACATCGTGACACGGTGCCTGAACCGGGTGTGCACCAATTACATGCCATATTTCT
TTACACTGCTGCTGCAGCTGTGCACCTTTACAAGGTCCACCAATTCTCGCATCAAG
GCCTCCATGCCCACCACAATCGCCAAGAACACAGTGAAGAGCGTGGGCAAGTTCT
GCCTGGAGGCCTCCTTTAACTACCTGAAGTCCCCCAATTTCTCTAAGCTGATCAAC
ATCATCATCTGGTTTCTGCTGCTGAGCGTGTGCCTGGGCAGCCTGATCTATTCCACA
GCCGCCCTGGGCGTGCTGATGAGCAACCTGGGCATGCCTTCCTACTGCACCGGCTA
TCGGGAGGGCTACCTGAATAGCACCAACGTGACAATCGCCACCTACTGTACAGGC
TCTATCCCATGCAGCGTGTGCCTGTCCGGCCTGGATTCTCTGGACACCTATCCTTCC
CTGGAGACCATCCAGATCACAATCTCCTCTTTCAAGTGGGACCTGACCGCCTTTGG
CCTGGTGGCAGAGTGGTTCCTGGCCTATATCCTGTTTACAAGATTCTTTTACGTGCT
GGGCCTGGCCGCCATCATGCAGCTGTTCTTTAGCTACTTCGCCGTGCACTTTATCTC
TAATAGCTGGCTGATGTGGCTGATCATCAACCTGGTGCAGATGGCCCCCATCTCCG
CCATGGTGAGGATGTATATCTTCTTTGCCTCTTTCTACTACGTGTGGAAGAGCTACG
TGCACGTGGTGGACGGCTGCAATAGCTCCACCTGCATGATGTGCTACAAGAGGAA
CCGCGCCACACGCGTGGAGTGTACCACAATCGTGAATGGCGTGCGGAGAAGCTTC
TACGTGTATGCCAACGGCGGCAAGGGCTTTTGCAAGCTGCACAACTGGAATTGCG
TGAACTGTGATACATTCTGTGCCGGCAGCACCTTTATCTCCGATGAGGTGGCAAGG
GACCTGTCCCTGCAGTTCAAGAGACCAATCAATCCCACCGATCAGTCTAGCTACAT
CGTGGACTCCGTGACAGTGAAGAACGGCTCTATCCACCTGTATTTCGATAAGGCCG
GCCAGAAGACATACGAGAGGCACTCCCTGTCTCACTTTGTGAATCTGGACAACCT
GCGCGCCAACAATACCAAGGGCAGCCTGCCCATCAACGTGATCGTGTTCGATGGC
AAGTCCAAGTGCGAGGAGTCCTCTGCCAAGAGCGCCTCCGTGTACTATAGCCAGC
TGATGTGCCAGCCTATCCTGCTGCTGGACCAGGCCCTGGTGTCCGATGTGGGCGAC
TCTGCCGAGGTGGCAGTGAAGATGTTTGATGCCTACGTGAATACCTTCAGCAGCAC
CTTCAACGTGCCAATGGAGAAGCTGAAGACCCTGGTGGCAACAGCAGAGGCAGA
GCTGGCCAAGAACGTGTCCCTGGACAATGTGCTGTCTACCTTCATCAGCGCCGCCC
GCCAGGGCTTTGTGGATTCTGACGTGGAGACAAAGGATGTGGTGGAGTGCCTGAA
GCTGAGCCACCAGTCCGATATCGAGGTGACCGGCGACAGCTGTAACAATTATATGC
TGACCTACAATAAGGTGGAGAACATGACACCCCGGGATCTGGGCGCCTGCATCGA
CTGTTCTGCCAGACACATCAATGCCCAGGTGGCCAAGAGCCACAATATCGCCCTGA
TCTGGAACGTGAAGGACTTCATGTCTCTGAGCGAGCAGCTGAGGAAGCAGATCCG
CTCCGCCGCCAAGAAGAACAATCTGCCCTTCAAGCTGACCTGCGCCACCACAAGG
CAGGTGGTGAACGTGGTCACCACAAAGATCGCCCTGAAGGGCGGCAAGATCGTG
AACAATTGGCTGAAGCAGCTGATCAAGGTGACCCTGGTGTTCCTGTTTGTGGCCG
CCATCTTCTACCTGATCACCCCCGTGCACGTGATGTCTAAGCACACAGATTTTTCTA
GCGAGATCATCGGCTATAAGGCCATCGACGGAGGAGTGACCAGGGATATCGCCAG
CACCGACACATGCTTCGCCAATAAGCACGCCGATTTCGACACCTGGTTTAGCCAGA
GGGGCGGCTCCTACACAAACGACAAGGCCTGTCCACTGATCGCAGCCGTGATCAC
CAGGGAAGTGGGATTCGTGGTGCCTGGACTGCCAGGAACAATCCTGAGGACCACA
AATGGCGACTTCCTGCACTTTCTGCCTCGCGTGTTTTCCGCCGTGGGCAACATCTG
CTATACCCCATCTAAGCTGATCGAGTACACCGATTTCGCCACATCCGCCTGCGTGCT
GGCCGCAGAGTGTACCATCTTTAAGGATGCCTCTGGCAAGCCCGTGCCTTACTGTT
ATGACACAAATGTGCTGGAGGGCTCTGTGGCCTATGAGAGCCTGCGGCCAGATAC
CAGATACGTGCTGATGGACGGCAGCATCATCCAGTTCCCCAACACATATCTGGAGG
GCTCTGTGCGGGTGGTGACCACATTTGACAGCGAGTACTGCCGGCACGGCACCTG
TGAGAGATCTGAGGCCGGCGTGTGCGTGTCCACATCTGGCAGGTGGGTGCTGAAC
AATGATTACTATCGCAGCCTGCCTGGCGTGTTCTGTGGCGTGGACGCCGTGAATCT
GCTGACCAACATGTTTACACCTCTGATCCAGCCAATCGGCGCCCTGGATATCAGCG
CCTCCATCGTGGCAGGAGGAATCGTGGCAATCGTGGTGACATGCCTGGCCTACTAT
TTCATGCGGTTCCGGAGGGCCTTCGGCGAGTACTCTCACGTGGTGGCCTTTAATAC
CCTGCTGTTCCTGATGAGCTTCACCGTGCTGTGCCTGACCCCCGTGTATAGCTTCCT
GCCTGGCGTGTACTCCGTGATCTACCTGTATCTGACCTTCTACCTGACAAACGACG
TGAGCTTTCTGGCCCACATCCAGTGGATGGTCATGTTCACCCCCCTGGTGCCTTTTT
GGATCACAATCGCCTATATCATCTGCATCTCCACCAAGCACTTCTATTGGTTCTTTTC
TAATTACCTGAAGCGGAGAGTGGTGTTTAACGGCGTGTCTTTCAGCACCTTTGAGG
AGGCCGCCCTGTGCACATTCCTGCTGAACAAGGAGATGTACCTGAAGCTGCGGTC
CGACGTGCTGCTGCCACTGACCCAGTACAATAGATATCTGGCCCTGTATAACAAGT
ACAAGTATTTCTCTGGCGCCATGGATACCACAAGCTACAGAGAGGCAGCATGCTGT
CACCTGGCAAAGGCCCTGAATGATTTTTCCAACTCTGGCAGCGACGTGCTGTACCA
GCCCCCTCAGACCTCTATCACAAGCGCCGTGCTGCAGTAACTAGCATAACCCCTTG
GGGCCTCTAAACGGGTCTTGAGGGGTTTTTTGTCTAGA.
The nucleotide sequence of ps2AC was shown in SEQ ID NO: 18:
(SEQ ID NO: 18)
GCTAGCGAGGGCCCGGAAACCTGGCCCTGTCTTCTTGACGAGCATTCCTAGG
GGTCTTTCCCCTCTCGCCAAAGGAATGCAAGGTCTGTTGAATGTCGTGAAGGAAG
CAGTTCCTCTGGAAGCTTCTTGAAGACAAACAACGTCTGTAGCGACCCTTTGCAG
GCAGCGGAACCCCCCACCTGGCGACAGGTGCCTCTGCGGCCAAAAGCCACGTGT
ATAAGATACACCTGCAAAGGCGGCACAACCCCAGTGCCACGTTGTGAGTTGGATA
GTTGTGGAAAGAGTCAAATGGCTCTCCTCAAGCGTATTCAACAAGGGGCTGAAGG
ATGCCCAGAAGGTACCCCATTGTATGGGATCTGATCTGGGGCCTCGGTGCACATGC
TTTACATGTGTTTAGTCGAGGTTAAAAAAACGTCTAGGCCCCCCGAACCACGGGG
ACGTGGTTTTCCTTTGAAAAACACGATGATAAATGAGCGGCTTTCGGAAGATGGCA
TTCCCATCCGGCAAGGTGGAGGGATGCATGGTGCAGGTGACATGTGGCACCACAA
CCCTGAATGGCCTGTGGCTGGACGATGTGGTGTATTGCCCTAGACACGTGATCTGT
ACCAGCGAGGACATGCTGAACCCAAATTACGAGGATCTGCTGATCAGGAAGTCCA
ACCACAATTTCCTGGTGCAGGCAGGAAACGTGCAGCTGCGCGTGATCGGCCACAG
CATGCAGAATTGCGTGCTGAAGCTGAAGGTGGACACAGCCAACCCAAAGACCCCC
AAGTACAAGTTTGTGAGGATCCAGCCTGGCCAGACATTCTCCGTGCTGGCCTGCTA
TAACGGCTCTCCCAGCGGCGTGTACCAGTGTGCCATGCGCCCTAACTTTACCATCA
AGGGCTCTTTCCTGAATGGCAGCTGCGGCTCCGTGGGCTTTAACATCGACTATGAT
TGCGTGAGCTTCTGTTACATGCACCACATGGAGCTGCCAACAGGAGTGCACGCAG
GAACCGACCTGGAGGGAAACTTCTACGGCCCCTTCGTGGACAGGCAGACCGCAC
AGGCAGCAGGCACAGATACAACCATCACCGTGAACGTGCTGGCCTGGCTGTACGC
CGCCGTGATCAACGGCGACCGGTGGTTTCTGAATAGATTCACAACCACACTGAAC
GATTTCAATCTGGTGGCCATGAAGTACAACTATGAGCCACTGACACAGGACCACGT
GGATATCCTGGGACCACTGAGCGCCCAGACCGGAATCGCCGTGCTGGACATGTGC
GCCTCCCTGAAGGAGCTGCTGCAGAACGGCATGAATGGAAGGACAATCCTGGGAA
GCGCCCTGCTGGAGGACGAGTTTACCCCATTCGATGTGGTGAGACAGTGTTCCGG
CGTGACATTTCAGGCCACCAATTTCTCTCTGCTGAAGCAGGCAGGCGATGTGGAG
GAGAACCCTGGACCATCCGCCGTGAAGCGCACAATCAAGGGCACCCACCACTGGC
TGCTGCTGACAATCCTGACCTCTCTGCTGGTGCTGGTGCAGTCTACCCAGTGGAGC
CTGTTCTTTTTCCTGTATGAGAATGCCTTTCTGCCCTTCGCCATGGGCATCATCGCC
ATGTCCGCCTTTGCCATGATGTTCGTGAAGCACAAGCACGCCTTTCTGTGCCTGTT
CCTGCTGCCATCCCTGGCCACCGTGGCCTACTTCAACATGGTGTATATGCCTGCCTC
TTGGGTCATGAGGATCATGACATGGCTGGACATGGTGGATACCTCCCTGTCTGGCT
TTAAGCTGAAGGACTGCGTGATGTATGCCAGCGCCGTGGTGCTGCTGATCCTGATG
ACAGCAAGGACCGTGTACGACGATGGAGCAAGGAGAGTGTGGACACTGATGAAT
GTGCTGACCCTGGTGTACAAGGTGTACTATGGCAACGCCCTGGATCAGGCCATCTC
CATGTGGGCCCTGATCATCTCTGTGACCAGCAATTATTCCGGCGTGGTGACCACAG
TGATGTTTCTGGCCCGGGGCATCGTGTTCATGTGCGTGGAGTACTGTCCTATCTTTT
TCATCACAGGCAACACCCTGCAGTGCATCATGCTGGTGTACTGTTTTCTGGGCTATT
TCTGCACCTGTTACTTTGGCCTGTTCTGCCTGCTGAATAGGTATTTTCGCCTGACAC
TGGGCGTGTACGACTATCTGGTGTCTACCCAGGAGTTCAGATACATGAACAGCCAG
GGCCTGCTGCCCCCTAAGAACTCCATCGATGCCTTCAAGCTGAATATCAAGCTGCT
GGGCGTGGGCGGCAAGCCATGCATCAAGGTGGCCACAGTGCAGTCTAAGATGAGC
GACGTGAAGTGTACCAGCGTGGTGCTGCTGTCCGTGCTGCAGCAGCTGAGGGTGG
AGAGCTCCTCTAAGCTGTGGGCCCAGTGCGTGCAGCTGCACAACGACATCCTGCT
GGCCAAGGATACCACAGAGGCCTTCGAGAAGATGGTGTCCCTGCTGTCTGTGCTG
CTGAGCATGCAGGGCGCCGTGGACATCAATAAGCTGTGCGAGGAGATGCTGGATA
ACCGCGCCACACTGCAGGCCATCGCCTCTGAGTTTAGCTCCCTGCCAAGCTATGCA
GCCTTCGCCACCGCACAGGAGGCATACGAGCAGGCCGTGGCCAATGGCGACTCCG
AGGTGGTGCTGAAGAAGCTGAAGAAGAGCCTGAACGTGGCCAAGTCCGAGTTCG
ACCGGGATGCCGCCATGCAGAGAAAGCTGGAGAAGATGGCCGACCAGGCCATGA
CACAGATGTATAAGCAGGCCAGGTCTGAGGATAAGCGCGCCAAGGTGACCAGCGC
CATGCAGACAATGCTGTTTACCATGCTGCGGAAGCTGGACAATGATGCCCTGAACA
ATATCATCAACAATGCCAGAGACGGCTGCGTGCCCCTGAACATCATCCCTCTGACC
ACAGCCGCCAAGCTGATGGTGGTCATCCCTGACTACAACACATATAAGAATACCTG
TGATGGCACCACATTCACATACGCCTCTGCCCTGTGGGAGATCCAGCAGGTGGTGG
ACGCCGATAGCAAGATCGTGCAGCTGAGCGAGATCTCCATGGATAACTCCCCAAAT
CTGGCATGGCCACTGATCGTGACCGCCCTGAGGGCCAATAGCGCCGTGAAGCTGC
AGAACAATGAGCTGTCCCCAGTGGCCCTGAGGCAGATGTCTTGCGCAGCAGGAAC
CACACAGACAGCCTGTACCGACGATAACGCCCTGGCCTACTATAATACCACAAAGG
GAGGCCGGTTTGTGCTGGCCCTGCTGTCTGACCTGCAGGATCTGAAGTGGGCCAG
ATTCCCTAAGAGCGACGGCACCGGCACAATCTACACCGAGCTGGAGCCACCCTGC
CGGTTTGTGACCGATACACCTAAGGGCCCAAAGGTGAAGTACCTGTATTTCATCAA
GGGCCTGAACAATCTGAACAGGGGAATGGTGCTGGGATCTCTGGCCGCAACCGTG
CGCCTGCAGGCAGGAAACGCCACAGAGGTGCCCGCCAATTCCACCGTGCTGTCTT
TTTGTGCCTTCGCCGTGGACGCAGCAAAGGCATACAAGGATTATCTGGCCTCCGGC
GGCCAGCCTATCACCAATTGCGTGAAGATGCTGTGCACCCACACAGGAACCGGAC
AGGCCATCACAGTGACCCCAGAGGCCAACATGGACCAGGAGTCTTTTGGCGGCGC
CAGCTGCTGTCTGTATTGCCGGTGTCACATCGACCACCCCAATCCTAAGGGCTTCT
GCGATCTGAAGGGCAAGTACGTGCAGATCCCTACCACATGTGCCAATGATCCAGTG
GGCTTTACCCTGAAGAACACAGTGTGCACCGTGTGCGGCATGTGGAAGGGCTACG
GCTGCAGCTGTGACCAGCTGAGAGAGCCCATGCTGCAGTCCGCCGATGCCCAGTC
TTTTCTGAACGGCTTCGCCGTGTAACTAGCATAACCCCTTGGGGCCTCTAAACGGG
TCTTGAGGGGTTTTTTGTCTAGA.
The nucleotide sequence of ps2B was shown in SEQ ID NO: 19.
SEQ ID NO: 19
GCTAGCGAGGGCCCGGAAACCTGGCCCTGTCTTCTTGACGAGCATTCCTAGG
GGTCTTTCCCCTCTCGCCAAAGGAATGCAAGGTCTGTTGAATGTCGTGAAGGAAG
CAGTTCCTCTGGAAGCTTCTTGAAGACAAACAACGTCTGTAGCGACCCTTTGCAG
GCAGCGGAACCCCCCACCTGGCGACAGGTGCCTCTGCGGCCAAAAGCCACGTGT
ATAAGATACACCTGCAAAGGCGGCACAACCCCAGTGCCACGTTGTGAGTTGGATA
GTTGTGGAAAGAGTCAAATGGCTCTCCTCAAGCGTATTCAACAAGGGGCTGAAGG
ATGCCCAGAAGGTACCCCATTGTATGGGATCTGATCTGGGGCCTCGGTGCACATGC
TTTACATGTGTTTAGTCGAGGTTAAAAAAACGTCTAGGCCCCCCGAACCACGGGG
ACGTGGTTTTCCTTTGAAAAACACGATGATAAATGTCAGCAGATGCACAATCATTT
CTTAACAGAGTGTGCGGAGTGTCAGCAGCAAGACTTACACCTTGCGGAACAGGAA
CATCAACAGATGTAGTTTATAGGGCCTTCGATATCTACAACGATAAAGTGGCAGGAT
TTGCAAAGTTCTTAAAGACCAATTGCTGCAGATTTCAAGAGAAGGACGAGGATGA
TAACCTTATCGATTCATACTTTGTGGTGAAGAGGCATACATTCAGCAATTACCAACA
CGAAGAAACAATCTACAACCTTCTTAAAGATTGCCCTGCAGTGGCAAAGCATGAC
TTCTTCAAGTTCAGAATCGATGGAGATATGGTGCCTCACATCTCAAGACAAAGACT
TACAAAGTATACGATGGCAGATCTCGTTTATGCGTTGCGCCATTTCGACGAGGGTA
ATTGTGACACCCTGAAGGAGATCCTGGTCACGTATAATTGCTGCGATGATGATTACT
TTAACAAGAAGGACTGGTATGATTTCGTAGAGAATCCTGACATTCTTAGAGTGTAC
GCAAACCTTGGAGAAAGAGTGAGACAAGCACTCCTAAAGACAGTTCAATTCTGCG
ACGCAATGAGAAACGCAGGAATCGTGGGAGTGCTTACACTTGATAACCAAGATCT
TAACGGAAACTGGTATGACTTTGGCGACTTTATACAGACAACACCTGGATCAGGAG
TGCCTGTGGTGGATTCATATTATAGCCTGCTGATGCCTATCCTTACACTTACAAGAG
CACTTACAGCAGAATCACATGTGGATACCGACTTGACCAAACCCTATATTAAATGG
GATCTGCTGAAATATGACTTTACAGAAGAACGACTTAAACTCTTCGACAGATACTT
TAAATACTGGGATCAAACATACCACCCTAACTGCGTGAACTGCCTTGATGATAGAT
GCATCCTTCACTGCGCAAACTTTAACGTGCTGTTCTCGACCGTGTTTCCTCCTACAT
CATTTGGACCTCTTGTGAGAAAGATCTTTGTGGACGGAGTACCTTTCGTCGTATCA
ACAGGATACCACTTTAGAGAACTTGGAGTAGTGCATAATCAAGATGTGAACCTACA
TTCTAGCCGATTATCATTTAAAGAACTTCTGGTTTATGCCGCGGACCCTGCAATGCA
CGCAGCAAGTGGCAATTTATTACTTGACAAACGGACAACCTGTTTCTCGGTTGCCG
CACTTACAAACAATGTAGCTTTCCAGACCGTAAAGCCAGGGAATTTCAACAAAGA
TTTCTATGACTTCGCCGTATCAAAGGGATTCTTCAAGGAGGGATCATCAGTGGAAC
TTAAACACTTCTTCTTCGCCCAGGATGGAAACGCAGCAATCTCAGATTACGATTAC
TACAGATACAACCTTCCTACAATGTGCGATATCAGACAACTTCTCTTCGTAGTTGAA
GTGGTGGATAAATACTTTGATTGCTACGATGGAGGATGCATCAACGCAAACCAAGT
GATCGTGAACAACTTGGATAAATCCGCTGGATTCCCGTTTAATAAGTGGGGTAAAG
CCCGCCTTTACTACGATTCAATGTCATACGAAGATCAAGATGCATTATTCGCTTATAC
AAAGAGGAATGTGATCCCTACAATCACACAAATGAACCTTAAATACGCAATCTCAG
CAAAGAATCGAGCAAGAACAGTGGCAGGAGTGTCAATCTGCTCAACAATGACAA
ACAGACAATTTCACCAGAAGCTCCTGAAATCAATCGCAGCAACAAGAGGAGCAAC
AGTGGTGATCGGAACATCAAAGTTCTATGGAGGTTGGCACAACATGCTCAAGACC
GTGTATAGCGATGTTGAGAATCCGCATCTCATGGGATGGGATTACCCTAAATGCGAT
AGAGCTATGCCCAATATGCTGAGAATCATGGCATCACTTGTGCTTGCAAGAAAGCA
TACCACATGCTGCTCACTTTCACACAGATTCTATCGACTTGCAAACGAATGCGCAC
AGGTCCTCTCCGAGATGGTGATGTGCGGCGGGAGCTTGTATGTGAAACCAGGTGG
AACATCATCAGGAGATGCAACAACAGCATACGCAAACTCAGTGTTTAACATCTGCC
AAGCAGTGACAGCTAATGTAAACGCTCTCTTGAGCACTGACGGAAACAAGATAGC
CGATAAATACGTGCGTAATCTGCAGCATCGACTTTACGAATGCCTTTACAGAAACA
GAGATGTAGACACGGACTTTGTAAATGAATTCTATGCTTACCTTAGAAAGCATTTCT
CCATGATGATACTGAGTGACGATGCTGTTGTATGTTTCAACTCAACATACGCATCAC
AAGGACTTGTGGCATCAATCAAGAATTTCAAATCAGTGCTTTACTACCAGAATAAT
GTGTTTATGTCAGAAGCAAAGTGTTGGACAGAAACTGACCTCACTAAGGGCCCTC
ACGAGTTCTGTAGCCAACACACAATGCTTGTGAAACAAGGAGATGACTATGTTTAT
CTCCCATACCCTGATCCTTCAAGAATCTTGGGTGCAGGGTGTTTCGTGGATGATATC
GTGAAGACTGACGGAACACTTATGATCGAAAGATTTGTGTCACTTGCAATCGATGC
ATACCCTCTTACAAAGCATCCGAACCAAGAATACGCAGATGTGTTTCACCTTTACCT
TCAATACATCAGAAAGTTGCATGATGAACTTACAGGACACATGCTTGATATGTACTC
AGTGATGCTTACAAACGATAACACATCAAGATACTGGGAACCTGAATTCTATGAGG
CAATGTACACACCTCACACAGTGCTTCAAGCAGTGGGAGCATGCGTGCTTTGCAA
CTCACAAACATCACTTAGATGCGGAGCATGCATCAGAAGACCTTTCCTGTGTTGCA
AATGCTGCTACGATCACGTGATCTCAACATCACACAAACTTGTGCTTTCAGTGAAC
CCTTACGTGTGCAACGCACCAGGCTGTGACGTAACTGACGTTACGCAGCTCTATCT
TGGAGGAATGTCATACTACTGCAAATCACACAAACCTCCTATCTCATTTCCTCTTTG
CGCAAACGGACAAGTGTTTGGACTTTACAAGAATACTTGCGTGGGATCAGATAAC
GTGACAGATTTCAATGCTATCGCAACATGCGATTGGACAAACGCAGGAGATTACAT
CCTTGCAAACACATGCACAGAGCGTCTGAAGTTGTTTGCGGCCGAAACACTTAAA
GCAACAGAAGAAACATTTAAACTTTCATACGGAATCGCAACAGTGAGAGAGGTCC
TATCGGACAGGGAACTCCACCTTTCATGGGAAGTGGGCAAACCACGCCCGCCGCT
TAACAGAAACTACGTGTTTACAGGATACAGAGTGACAAAGAATTCTAAGGTACAG
ATCGGAGAATACACATTTGAGAAGGGCGACTACGGAGACGCCGTGGTGTACAGAG
GGACGACTACGTATAAACTTAACGTGGGAGATTACTTTGTGCTTACATCACACACA
GTGATGCCTCTTTCAGCACCTACACTTGTGCCTCAAGAGCATTATGTCCGAATAAC
GGGTCTCTATCCGACACTTAACATCTCAGATGAATTCTCGAGTAACGTGGCAAACT
ACCAGAAAGTGGGTATGCAGAAATACTCCACCTTACAGGGACCTCCTGGTACAGG
AAAGTCTCATTTCGCGATAGGTCTAGCTCTCTATTACCCTTCAGCAAGAATCGTGTA
CACAGCATGCTCACACGCAGCAGTGGATGCACTTTGCGAGAAGGCGCTGAAATAC
CTTCCTATCGATAAATGCTCAAGAATCATCCCTGCAAGAGCAAGAGTGGAATGCTT
TGATAAATTTAAAGTGAACTCAACACTTGAACAATACGTGTTCTGTACTGTAAATG
CTCTGCCTGAAACTACCGCGGATATCGTGGTGTTCGACGAGATATCCATGGCAACA
AACTACGACCTATCGGTCGTAAACGCGCGGCTAAGAGCAAAGCATTATGTGTACAT
CGGAGATCCTGCACAACTTCCTGCACCTAGAACATTACTAACTAAAGGGACGCTCG
AACCTGAATACTTTAACAGTGTTTGTCGCCTAATGAAGACGATCGGGCCGGACATG
TTTCTTGGAACATGCAGAAGATGCCCTGCAGAAATCGTGGATACAGTGTCAGCACT
TGTGTACGATAACAAACTTAAAGCACACAAAGACAAGTCGGCTCAGTGTTTCAAG
ATGTTTTACAAAGGAGTGATCACACACGATGTGTCATCAGCAATCAACAGACCTCA
AATCGGAGTGGTGAGAGAATTTCTTACAAGAAACCCTGCATGGAGAAAGGCGGTC
TTCATAAGTCCTTACAACTCACAGAATGCCGTGGCATCAAAGATACTCGGGCTTCC
TACACAAACAGTGGATTCATCACAAGGATCAGAATACGATTACGTGATCTTTACAC
AAACAACAGAAACAGCACACTCATGCAACGTGAACAGATTTAACGTGGCAATCAC
AAGAGCAAAGGTAGGGATCCTCTGTATCATGTCAGATAGAGATCTTTACGATAAAC
TTCAATTTACATCACTTGAAATCCCTAGAAGAAACGTGGCGACTCTGCAGGCTGAG
AACGTGACAGGATTGTTCAAGGACTGCTCAAAGGTAATTACGGGTTTACATCCGAC
ACAAGCACCTACACACCTTTCAGTGGATACAAAGTTCAAGACTGAAGGACTTTGC
GTGGATATCCCTGGAATCCCTAAAGATATGACATACAGAAGACTTATCTCAATGATG
GGATTTAAGATGAATTACCAAGTGAACGGATACCCTAACATGTTTATCACAAGAGA
AGAAGCAATCAGACACGTGAGAGCATGGATAGGCTTCGACGTCGAGGGATGCCAC
GCAACAAGAGAAGCAGTGGGAACAAACCTTCCTCTTCAACTTGGATTCTCCACTG
GAGTGAACCTTGTGGCAGTGCCTACAGGATACGTGGATACACCTAACAACACAGA
TTTCTCGCGAGTGTCAGCAAAGCCACCACCTGGAGATCAATTTAAACACCTTATCC
CTCTTATGTACAAAGGACTTCCTTGGAACGTGGTGAGAATCAAGATAGTCCAAATG
CTATCCGATACCTTAAAGAATCTTAGTGACCGTGTCGTATTTGTGCTTTGGGCACAC
GGATTTGAACTTACATCAATGAAATACTTTGTGAAGATCGGTCCCGAGCGTACATG
CTGCCTTTGCGATAGAAGAGCTACGTGTTTCAGTACCGCTTCAGATACATACGCATG
CTGGCACCACTCAATAGGCTTCGATTACGTTTATAATCCGTTCATGATAGATGTGCA
ACAATGGGGATTCACGGGCAATCTGCAGAGCAACCACGATCTTTACTGCCAAGTG
CACGGAAACGCACACGTGGCATCATGCGATGCAATCATGACAAGATGCCTTGCAG
TGCACGAATGCTTTGTGAAGCGGGTCGATTGGACAATCGAATACCCTATCATCGGA
GATGAACTTAAGATAAATGCAGCATGCAGAAAGGTCCAGCACATGGTGGTGAAAG
CAGCACTTCTTGCAGATAAATTTCCTGTGCTTCACGATATCGGAAACCCTAAAGCA
ATCAAATGCGTGCCTCAAGCAGATGTGGAATGGAAATTCTATGACGCACAACCTTG
CTCAGATAAAGCATACAAGATAGAGGAACTATTCTATAGTTACGCAACACACTCAG
ATAAATTTACAGATGGAGTGTGCCTGTTCTGGAATTGCAACGTGGATAGATACCCTG
CAAACTCAATCGTGTGCAGATTTGATACAAGAGTGCTTTCAAACCTTAACCTTCCA
GGTTGTGACGGCGGCAGTCTATATGTTAATAAGCACGCATTTCACACACCTGCATTC
GATAAGTCCGCATTCGTCAATTTAAAGCAGCTACCTTTCTTCTATTATTCAGATTCAC
CTTGCGAATCACACGGAAAGCAGGTTGTCAGTGACATCGATTACGTGCCTCTTAAA
TCAGCAACATGTATTACCAGGTGTAATCTTGGAGGAGCCGTCTGTCGACATCATGC
AAACGAATACAGACTTTACCTTGATGCATACAACATGATGATCTCCGCCGGGTTCTC
CCTATGGGTGTACAAACAATTTGATACATACAACCTTTGGAACACATTTACAAGACT
TCAATCACTTGAGAACGTTGCGTTCAATGTAGTCAATAAGGGACACTTCGACGGTC
AACAGGGTGAGGTTCCTGTGTCAATCATCAACAATACCGTTTATACTAAAGTTGAC
GGCGTGGATGTGGAACTCTTCGAGAATAAGACTACGCTTCCTGTGAATGTTGCCTT
CGAGTTGTGGGCAAAGCGCAATATCAAACCTGTGCCTGAAGTGAAGATACTCAAT
AACCTTGGAGTGGATATCGCAGCAAACACAGTGATCTGGGATTACAAGAGGGACG
CACCTGCACACATCTCAACAATCGGAGTGTGCTCAATGACAGATATCGCAAAGAA
GCCGACTGAAACAATCTGCGCACCTCTTACTGTATTCTTCGACGGAAGAGTGGATG
GACAAGTGGATTTATTCCGAAATGCAAGAAACGGAGTGCTTATCACAGAAGGATC
AGTGAAAGGACTTCAACCTTCAGTGGGACCTAAACAAGCATCACTTAACGGAGTG
ACTCTGATAGGCGAGGCCGTGAAGACTCAGTTTAACTACTACAAGAAAGTAGACG
GTGTCGTCCAGCAGCTGCCCGAGACCTATTTCACACAATCACGGAATCTGCAGGA
GTTCAAACCTAGATCACAAATGGAAATCGATTTCCTGGAGCTTGCAATGGATGAAT
TTATCGAAAGATACAAACTTGAAGGATACGCATTTGAACACATCGTGTACGGAGAT
TTCAGTCATTCACAACTTGGAGGACTTCACCTTCTTATTGGCCTAGCCAAACGTTTC
AAAGAATCACCTTTCGAGCTCGAAGATTTCATTCCAATGGATTCAACAGTGAAGAA
TTATTTCATTACTGACGCCCAGACGGGATCATCAAAGTGTGTATGCTCAGTGATCGA
TCTACTACTAGACGATTTCGTTGAAATTATTAAATCACAAGACTTGAGTGTAGTTAG
TAAGGTTGTGAAGGTCACAATCGATTACACAGAAATCTCATTTATGCTTTGGTGCA
AAGATGGACACGTGGAAACATTCTATCCCAAACTTCAATCATCACAAGCATGGCAA
CCTGGAGTGGCCATGCCGAATTTGTATAAGATGCAGAGAATGCTTCTTGAGAAGTG
TGACCTTCAGAATTATGGAGATTCAGCAACACTTCCTAAAGGAATCATGATGAACG
TGGCAAAGTATACTCAACTTTGCCAATACCTTAACACACTTACACTTGCAGTGCCTT
ACAACATGAGAGTGATCCACTTCGGTGCAGGGTCGGACAAAGGAGTGGCACCTG
GTACTGCTGTCCTTAGACAATGGCTTCCTACAGGAACACTTCTTGTGGATTCAGAT
CTTAACGATTTCGTCTCCGATGCAGATTCAACCCTCATTGGTGACTGTGCAACAGT
GCACACAGCAAACAAGTGGGACTTAATAATATCAGATATGTACGATCCTAAGACTA
AGAATGTAACGAAAGAGAATGACTCAAAGGAAGGTTTCTTCACCTATATCTGCGG
ATTTATCCAACAGAAGTTAGCTCTTGGAGGATCAGTGGCAATCAAGATTACGGAAC
ACTCATGGAACGCAGATCTTTACAAACTTATGGGACACTTTGCATGGTGGACCGCG
TTCGTTACAAACGTAAACGCGTCGTCCTCAGAAGCATTTCTTATCGGATGCAACTA
CCTTGGGAAACCAAGAGAGCAGATCGATGGATACGTGATGCACGCAAACTACATC
TTCTGGAGGAACACAAACCCTATCCAACTTTCATCATACTCACTCTTCGACATGTCA
AAGTTCCCGCTTAAACTTAGAGGGACTGCCGTAATGTCGCTTAAAGAAGGACAAA
TCAACGATATGATACTCAGCCTCCTAAGTAAAGGGAGGCTTATCATCAGAGAGAAT
AATAGAGTGGTGATCTCATCAGATGTGCTTGTGAACAACTAACTAGCATAACCCCT
TGGGGCCTCTAAACGGGTCTTGAGGGGTTTTTTGTCTAGA.

(II) The expression structure comprising nucleotide sequences of 5′ UTR and 3′ UTR of novel coronavirus SARS-COV-2, a transcription regulatory region on which the novel coronavirus SARS-COV-2 non-structural protein could act, and a reporter gene.

Since the expression of S protein, ORF3a, M, ORF7a, ORF8, or N protein of novel coronavirus SARS-COV-2 depended on the participation of the 16 proteins, i.e., proteins nsps 1-16, which matured to form a viral transcriptase/replicase complex, and on the 5′ UTR sequence, 3′ UTR sequence, and transcription regulatory region (TRS) sequence in the viral genome, the transcription regulatory region (TRS) sequence in (II) could be at least one TRS sequences of S, ORF3a, M, ORF7a, ORF8, or N, and the core sequence (AAACGAAC) of the TRS region could be used either alone or in combination with other sequences. Since a transcription regulatory region on which the novel coronavirus SARS-COV-2 non-structural protein could act was connected upstream of reporter gene B, the expression of reporter gene B was dependent on an Nsp1-Nsp16 replicase/transcriptase complex formed by ps2AN, ps2AC, and ps2B transcription, translation and maturing.

The nucleotide sequence of the transcription regulatory region for S protein (S-TRS) was shown in SEQ ID NO: 20; the nucleic acid sequence of the transcription regulatory region for ORF3a protein (ORF3a-TRS) was shown in SEQ ID NO: 21: the nucleic acid sequence of the transcription regulatory region for protein M (M-TRS) was shown in SEQ ID NO: 22; the nucleic acid sequence of the transcription regulatory region for ORF7a protein (ORF7a-TRS) was shown in SEQ ID NO: 23: the nucleic acid sequence of the transcription regulatory region for ORF8 protein (ORF8-TRS) was shown in SEQ ID NO: 24; and the nucleic acid sequence of the transcription regulatory region for N protein (N-TRS) was shown in SEQ ID NO: 25.

(SEQ ID NO: 20)
AGTGATGTTCTTGTTAACAACTAAACGAACAATGTTTGTTTTTCTTGTT
T;
(SEQ ID NO: 21)
AGTCAAATTACATTACACATAAACGAACTTATGGATTTGTTTATGAGAA
T;
(SEQ ID NO: 22)
TGATCTTCTGGTCTAAACGAACTAAATATTATATTAGTTTTTCTGTTTG
GAACTTTAATTTTAGCC;
(SEQ ID NO: 23)
GCAACCAATGGAGATTGATTAAACGAACATGAAAATTATTCTTTTCTTG
G;
(SEQ ID NO: 24)
TTGAACTTTCATTAATTGACTTCTATTTGTGCTTTTTAGCCTTTCTGCT
ATTCCTTGTTTTAATTATGCTTATTATCTTTTGGTTCTCACTTGAACTG
CAAGATCATAATGAAACTTGTCACGCCTAAACGAAC;
and
(SEQ ID NO: 25)
TTTAGATTTCATCTAAACGAACAAACTAAAATGTCTGATAATGGACCCC
A.

In order to make the replicon system comprising the above-mentioned expression structure more accurate, an additional reporter gene was introduced into the expression structure (II) as a control.

Nucleic acid sequences of 5′ UTR of novel coronavirus SARS-COV-2, reporter gene A as a control, a transcription regulatory region on which the novel coronavirus SARS-COV-2 non-structural protein could act, reporter gene B, and 3′ UTR of novel coronavirus SARS-COV-2 were connected in the expression structure in order, wherein the reporter gene A and the reporter gene B were different types of reporter genes. For example, the reporter gene A was fluorescent protein and the reporter gene B was luciferase.

A nucleic acid sequence of a ribosome entry site (IRES) was further connected between the 5′ UTR of novel coronavirus SARS-COV-2 and reporter gene A. A translation stop codon was inserted at an end of reporter gene A.

In this example, reporter gene A was green fluorescent protein (GFP) with four stop codons inserted at an end; reporter gene B was luciferase; and the TRS sequence was a transcription regulatory region (M-TRS) sequence for M protein.

The nucleotide sequence of the 5′ UTR of novel coronavirus SARS-CoV-2 was shown
in SEQ ID NO: 26:
(SEQ ID NO: 26)
ATTAAAGGTTTATACCTTCCCAGGTAACAAACCAACCAACTTTCGATCTCTTGT
AGATCTGTTCTCTAAACGAACTTTAAAATCTGTGTGGCTGTCACTCGGCTGCATGC
TTAGTGCACTCACGCAGTATAATTAATAACTAATTACTGTCGTTGACAGGACACGAG
TAACTCGTCTATCTTCTGCAGGCTGCTTACGGTTTCGTCCGTGTTGCAGCCGATCAT
CAGCACATCTAGGTTTCGTCCGGGTGTGACCGAAAGGTAAG.
The nucleotide sequence of the 3′ UTR of novel coronavirus SARS-CoV-2 was
shown in SEQ ID NO: 27:
(SEQ ID NO: 27)
TGGGCTATATAAACGTTTTCGCTTTTCCGTTTACGATATATAGTCTACTCTTGTG
CAGAATGAATTCTCGTAACTACATAGCACAAGTAGATGTAGTTAACTTTAATCTCAC
ATAGCAATCTTTAATCAGTGTGTAACATTAGGGAGGACTTGAAAGAGCCACCACAT
TTTCACCGAGGCCACGCGGAGTACGATCGAGTGTACAGTGAACAATGCTAGGGAG
AGCTGCCTATATGGAAGAGCCCTAATGTGTAAAATTAATTTTAGTAGTGCTATCCCC
ATGTGATTTTAATA.
The nucleotide sequence of the inserted ribosome entry site (IRES) was
preferably shown in SEQ ID NO: 28:
(SEQ ID NO: 28)
GAGGGCCCGGAAACCTGGCCCTGTCTTCTTGACGAGCATTCCTAGGGGTCTTT
CCCCTCTCGCCAAAGGAATGCAAGGTCTGTTGAATGTCGTGAAGGAAGCAGTTCC
TCTGGAAGCTTCTTGAAGACAAACAACGTCTGTAGCGACCCTTTGCAGGCAGCGG
AACCCCCCACCTGGCGACAGGTGCCTCTGCGGCCAAAAGCCACGTGTATAAGATA
CACCTGCAAAGGCGGCACAACCCCAGTGCCACGTTGTGAGTTGGATAGTTGTGGA
AAGAGTCAAATGGCTCTCCTCAAGCGTATTCAACAAGGGGCTGAAGGATGCCCAG
AAGGTACCCCATTGTATGGGATCTGATCTGGGGCCTCGGTGCACATGCTTTACATGT
GTTTAGTCGAGGTTAAAAAAACGTCTAGGCCCCCCGAACCACGGGGACGTGGTTT
TCCTTTGAAAAACACGATGATAA.
The nucleotide sequence of the inserted four stop codons was preferably shown
in SEQ ID NO: 29:
(SEQ ID NO: 29)
TAATAATAATAA.

In this example, the 5′ end of the Ps2V molecule was the non-coding region 5′-UTR at the 5′ end of SARS-COV-2; downstream was a ribosome entry site (IRES); further downstream was GFP reporter gene, wherein four translation stop codons were inserted at an end of the GFP reporter gene; further downstream was firefly luciferase gene connected to the transcription regulatory region (TRS) for M protein of SARS-COV-2; and the 3′ end was the non-coding region 3′-UTR at the 3′ end of SARS-COV-2.

Finally, the expression structure ps2V was constructed. The nucleotide sequence
of ps2V was shown in SEQ ID NO: 30:
(SEQ ID NO: 30)
GCTAGCATTAAAGGTTTATACCTTCCCAGGTAACAAACCAACCAACTTTCGAT
CTCTTGTAGATCTGTTCTCTAAACGAACTTTAAAATCTGTGTGGCTGTCACTCGGCT
GCATGCTTAGTGCACTCACGCAGTATAATTAATAACTAATTACTGTCGTTGACAGGA
CACGAGTAACTCGTCTATCTTCTGCAGGCTGCTTACGGTTTCGTCCGTGTTGCAGC
CGATCATCAGCACATCTAGGTTTCGTCCGGGTGTGACCGAAAGGTAAGGTGGAGA
GCCTTGTCCCTGGTTTCAACGAGAAAACACACGTCCAACTCAGTTTGCCTGTTTTA
CAGGTTCGCGACGTGCTCGTACGTGGCTTTGGAGACTCCGTGGAGGAGGTCTTATC
AGAGGCACGTCAACATCTTAAAGATGGCACTTGTGGCTTAGTAGAAGTTGAAAAA
GGCGTTTTGCCTCAACTTGAACAGCCTGAGCTTTGGGCTAAGCGCAACATTAAACC
AGTACCAGAGGTGAAAATACTCAATAATTTGGGTGTGGACATTGCTGCTAATACTG
TGATCTGGGACTACAAAAGAGATGCTCCAGCACATATATCTACTATTGGTGTTTGTT
CTATGACTGACATAGCCAAGAAACCAACTGAAACGATTTGTGCACCACTCACTGTC
TTTTTTGATGGTAGAGTTGATGGTCAAGTAGACTTATTTAGAAATGCCCGTAATGGT
GTTCTTATTACAGAAGGTAGTGTTAAAGGTTTACAACCATCTGTAGGTCCCAAACA
AGCTAGTCTTAATGGAGTCACATTAATTGGAGAAGCCGTAAAAACACAGTTCAATT
ATTATAAGAAAGTTGATGGTGTTGTCCAACAATTACCTGAAACTTACTTTACTCAGA
GTAGAAATTTACAAGAATTTAAACCCAGGAGTCAAATGGAAATTGATTTCTTAGAA
TTAGCTATGGATGAATTCATTGAACGGTATAAATTAGAAGGCTATGCCTTCGAACAT
ATCGTTTATGGAGATTTTAGTCATGAGGGCCCGGAAACCTGGCCCTGTCTTCTTGA
CGAGCATTCCTAGGGGTCTTTCCCCTCTCGCCAAAGGAATGCAAGGTCTGTTGAAT
GTCGTGAAGGAAGCAGTTCCTCTGGAAGCTTCTTGAAGACAAACAACGTCTGTAG
CGACCCTTTGCAGGCAGCGGAACCCCCCACCTGGCGACAGGTGCCTCTGCGGCCA
AAAGCCACGTGTATAAGATACACCTGCAAAGGCGGCACAACCCCAGTGCCACGTT
GTGAGTTGGATAGTTGTGGAAAGAGTCAAATGGCTCTCCTCAAGCGTATTCAACAA
GGGGCTGAAGGATGCCCAGAAGGTACCCCATTGTATGGGATCTGATCTGGGGCCTC
GGTGCACATGCTTTACATGTGTTTAGTCGAGGTTAAAAAAACGTCTAGGCCCCCCG
AACCACGGGGACGTGGTTTTCCTTTGAAAAACACGATGATAAGCGGCCGCATGGT
GAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGCGAGGGCGATGC
CACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTA
CCCCGACCACATGAAGCAGCACGACTTCTTCAAGTCCGCCATGCCCGAAGGCTAC
GTCCAGGAGCGCACCATCTTCTTCAAGGACGACGGCAACTACAAGACCCGCGCCG
AGGTGAAGTTCGAGGGCGACACCCTGGTGAACCGCATCGAGCTGAAGGGCATCG
ACTTCAAGGAGGACGGCAACATCCTGGGGCACAAGCTGGAGTACAACTACAACA
GCCACAACGTCTATATCATGGCCGACAAGCAGAAGAACGGCATCAAGGTGAACTT
CAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCT
GCTGGAGTTCGTGACCGCCGCCGGGATCACTCTCGGCATGGACGAGCTGTACAAG
TAATAATAATAAGATATCTGATCTTCTGGTCTAAACGAACTAAATATTATATTAGTTTT
TCTGTTTGGAACTTTAATTTTAGCCATGGCCGATGCTAAGAACATTAAGAAGGGCC
CTGCTCCCTTCTACCCTCTGGAGGATGGCACCGCTGGCGAGCAGCTGCACAAGGC
CATGAAGAGGTATGCCCTGGTGCCTGGCACCATTGCCTTCACCGATGCCCACATTG
AGGTGGACATCACCTATGCCGAGTACTTCGAGATGTCTGTGCGCCTGGCCGAGGCC
ATGAAGAGGTACGGCCTGAACACCAACCACCGCATCGTGGTGTGCTCTGAGAACT
CTCTGCAGTTCTTCATGCCAGTGCTGGGCGCCCTGTTCATCGGAGTGGCCGTGGCC
CCTGCTAACGACATTTACAACGAGCGCGAGCTGCTGAACAGCATGGGCATTTCTCA
GCCTACCGTGGTGTTCGTGTCTAAGAAGGGCCTGCAGAAGATCCTGAACGTGCAG
AAGAAGCTGCCTATCATCCAGAAGATCATCATCATGGACTCTAAGACCGACTACCA
GGGCTTCCAGAGCATGTACACATTCGTGACATCTCATCTGCCTCCTGGCTTCAACG
AGTACGACTTCGTGCCAGAGTCTTTCGACAGGGACAAAACCATTGCCCTGATCATG
AACAGCTCTGGGTCTACCGGCCTGCCTAAGGGCGTGGCCCTGCCTCATCGCACCG
CCTGTGTGCGCTTCTCTCACGCCCGCGACCCTATTTTCGGCAACCAGATCATCCCC
GACACCGCTATTCTGAGCGTGGTGCCATTCCACCACGGCTTCGGCATGTTCACCAC
CCTGGGCTACCTGATTTGCGGCTTTCGGGTGGTGCTGATGTACCGCTTCGAGGAGG
AGCTGTTCCTGCGCAGCCTGCAAGACTACAAAATTCAGTCTGCCCTGCTGGTGCC
AACCCTGTTCAGCTTCTTCGCTAAGAGCACCCTGATCGACAAGTACGACCTGTCTA
ACCTGCACGAGATTGCCTCTGGCGGCGCCCCACTGTCTAAGGAGGTGGGCGAAGC
CGTGGCCAAGCGCTTTCATCTGCCAGGCATCCGCCAGGGCTACGGCCTGACCGAG
ACAACCAGCGCCATTCTGATTACCCCAGAGGGCGACGACAAGCCTGGCGCCGTGG
GCAAGGTGGTGCCATTCTTCGAGGCCAAGGTGGTGGACCTGGACACCGGCAAGA
CCCTGGGAGTGAACCAGCGCGGCGAGCTGTGTGTGCGCGGCCCTATGATTATGTCC
GGCTACGTGAATAACCCTGAGGCCACAAACGCCCTGATCGACAAGGACGGCTGGC
TGCACTCTGGCGACATTGCCTACTGGGACGAGGACGAGCACTTCTTCATCGTGGA
CCGCCTGAAGTCTCTGATCAAGTACAAGGGCTACCAGGTGGCCCCAGCCGAGCTG
GAGTCTATCCTGCTGCAGCACCCTAACATTTTCGACGCCGGAGTGGCCGGCCTGCC
CGACGACGATGCCGGCGAGCTGCCTGCCGCCGTCGTCGTGCTGGAACACGGCAAG
ACCATGACCGAGAAGGAGATCGTGGACTATGTGGCCAGCCAGGTGACAACCGCCA
AGAAGCTGCGCGGCGGAGTGGTGTTCGTGGACGAGGTGCCCAAGGGCCTGACCG
GCAAGCTGGACGCCCGCAAGATCCGCGAGATCCTGATCAAGGCTAAGAAAGGCG
GCAAGATCGCCGTGTAAGGATCCGTGGGCTATATAAACGTTTTCGCTTTTCCGTTTA
CGATATATAGTCTACTCTTGTGCAGAATGAATTCTCGTAACTACATAGCACAAGTAG
ATGTAGTTAACTTTAATCTCACATAGCAATCTTTAATCAGTGTGTAACATTAGGGAG
GACTTGAAAGAGCCACCACATTTTCACCGAGGCCACGCGGAGTACGATCGAGTGT
ACAGTGAACAATGCTAGGGAGAGCTGCCTATATGGAAGAGCCCTAATGTGTAAAAT
TAATTTTAGTAGTGCTATCCCCATGTGATTTTAATAGCTTCTTAGGAGAATGACAAA
AAAAAAAAAAAAAAAAAAAAAAAAAAAAAACTAGCATAACCCCTTGGGGCCTCT
AAACGGGTCTTGAGGGGTTTTTTGTCTAGA.

The replicon structures in (I) and (II) mentioned above were inserted into expression vectors to construct a replicon system comprising:
(i) a nucleic acid sequence encoding a novel coronavirus SARS-COV-2 non-structural protein; and
(ii) nucleic acid sequences of 5′ UTR and 3′ UTR of novel coronavirus SARS-COV-2, a transcription regulatory region on which the novel coronavirus SARS-COV-2 non-structural protein could act, and a reporter gene.

The expression vector could be an eukaryotic expression vector or a prokaryotic expression vector depending on the detection purpose.

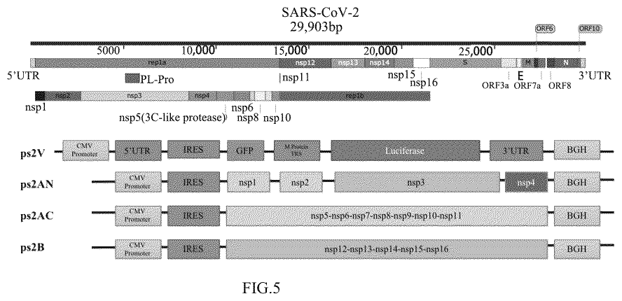

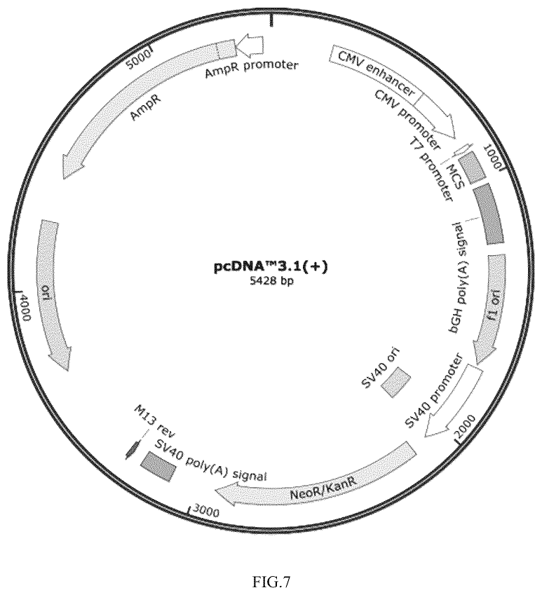

In this example, pcDNA3.1 plasmids were selected as expression vectors, and ps2V, ps2AN, ps2AC, and ps2B were respectively inserted into the pcDNA3.1 plasmids by means of double digestion with NheI and XbaI (the map of the plasmid was shown in FIG. 7) to construct four eukaryotic expression vectors (as shown in FIG. 5).

### Example 2 Establishment of Novel Coronavirus SARS-COV-2 Replicon System

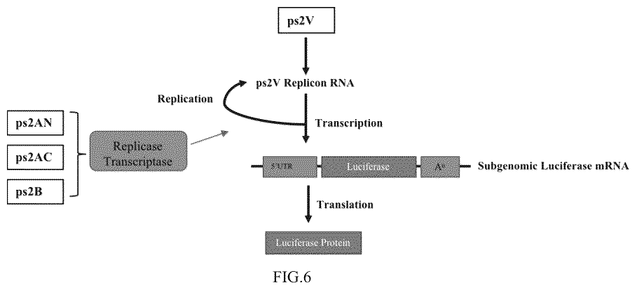

The purpose of the construction of the replicon system in Example 1 was to screen an anti-novel coronavirus SARS-COV-2 drug, especially a human drug, so HEK 293T cell line was used as a packaging cell for verification. The schematic diagram of the working principles of the four expression vectors, ps2V, ps2AN, ps2AC, and ps2B, in the human body or human cells was shown in FIG. 6.

HEK293T cells in a good growth state were evenly plated in a 12-well culture plate treated with polylysine (at a cell density of about 6.5×10⁴/cm²), and the cells were uniformly distributed individually. After about 24 h of culture, the cell confluence was close to 80%. At this point, an Opti-Lipo2000-DNA mixed liquid as shown in Table 1 was prepared and used for transfection.

The concentrations of the four vectors could be between 0.01 μg/μL and 1 μg/μL, and the ratio of the four vectors could be adjusted within the above range.

TABLE 1
Opti-Lipo2000-DNA mixed liquid system

| Plasmid | Plasmid | | | Lipo2000 |
|---|---|---|---|---|
| name | amount | | Opti amount | amount |
| Ps2AN | 0.05 | μg | 100 μl for dissolving plasmid and 100 μl | 2 μl |
| Ps2AC | 0.4 | μg | for dissolving lipo 2000; and in each | |
| Ps2B | 0.4 | μg | case, incubation for 5 min and mixing, | |
| Ps2V | 0.1 | μg | and then incubation for further 20 min, | |
| | | | followed by the addition of cells. | |

After transfection, the transfection effect could be evaluated by observing the expression of green fluorescent protein in the cells. As could be seen from FIG. 8A and FIG. 8B, transfection either with ps2V alone or with ps2V mixed with ps2AN, ps2AC, and ps2B plasmids had a high level of GFP expression, indicating that this transfection scheme enabled the ps2V plasmid to be effectively expressed. Since the expression level of GFP did not depend on the regulation by the transcription regulatory region (TRS) of SARS-COV-2, the expression of GFP was independent of ps2AN, ps2AC, and ps2B transfections.

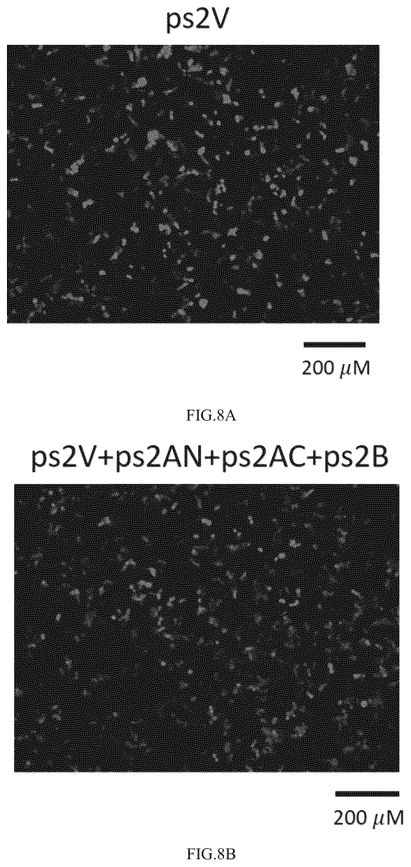

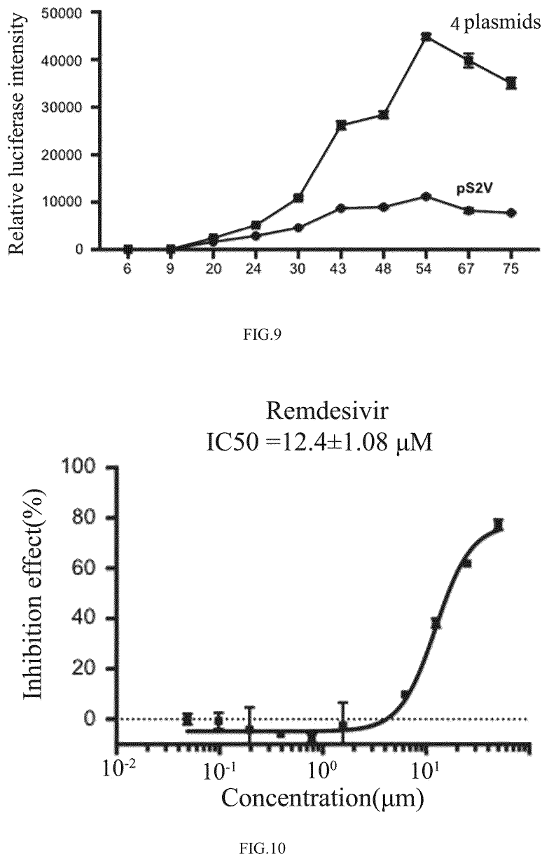

Subsequently, according to the detection time point, 200 μl of Promega cell lysate was added to the cells, the cells were repeatedly pipetted with a pipette, and the lysate was put into a 1.5 mL Ep tube and oscillated on an oscillator for 20 min at room temperature. The intracellular luciferase activities at different time points were detected by luciferase detection system, and the results were shown in FIG. 9. It could be seen that the cellular luciferase activity in cells co-transfected with ps2V, ps2AN, ps2AC, and ps2B reached its peak about 54 h after transfection and then gradually decreased. Whereas, the luciferase activity in cells transfected with ps2V alone was kept at a low level, which indicated that HEK293T cells could well support the replication and transcription of the novel coronavirus SARS-COV-2 safe replicon established by the present disclosure, and also demonstrated the effectiveness of the novel coronavirus SARS-COV-2 system established by the present disclosure. The results indicated that the replicon system constructed in Example 1 could achieve functions in packaging cells.

### Example 3 Detection of the Performance of the Novel Coronavirus SARS-COV-2 Replicon System

According to the steps in Example 2, transfection was carried out with ps2V, ps2AN, ps2AC, and ps2B plasmids. 6 h after transfection, Remdesivir, Lopinavir, and Ritonavir were added according to the concentration gradient (20 μM, 10 μM, 5 μM, 2.5 μM, 1.25 μM, 0.625 μM, 0.3125 μM, 0.15625 μM, 0.078125 μM, and 0.0390625 μM). After 24 h of drug treatment, the cellar luciferase activity was detected, the inhibition rate was calculated with DMSO control as a baseline, and the semi-inhibitory concentration (hereinafter referred to as IC50) of the drug was calculated using Graphpad Prism 7.0 software. The specific results were shown in FIGS. 10 to 12.

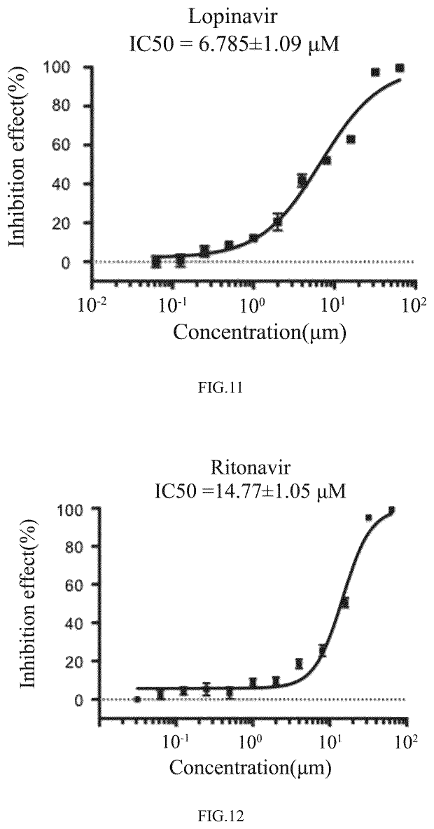

The results in FIG. 10 showed that the IC50 of Remdesivir was 12.4±1.08 μM: the results in FIG. 11 showed that the IC50 of Lopinavir was 6.785±1.09 μM; and the results in FIG. 12 showed that the IC50 of Ritonavir was 14.77=1.05 μM.

The results of the above data indicated that the replicon system constructed in Example 1 could reproduce the response of wild-type SARS-COV-2 to a drug, with a closer IC50, indicating that the constructed novel coronavirus SARS-COV-2 replicon system could highly simulate the response of wild-type SARS-COV-2 to a drug.

### Example 4 Drug Screening by Means of the Novel Coronavirus SARS-COV-2 Replicon System

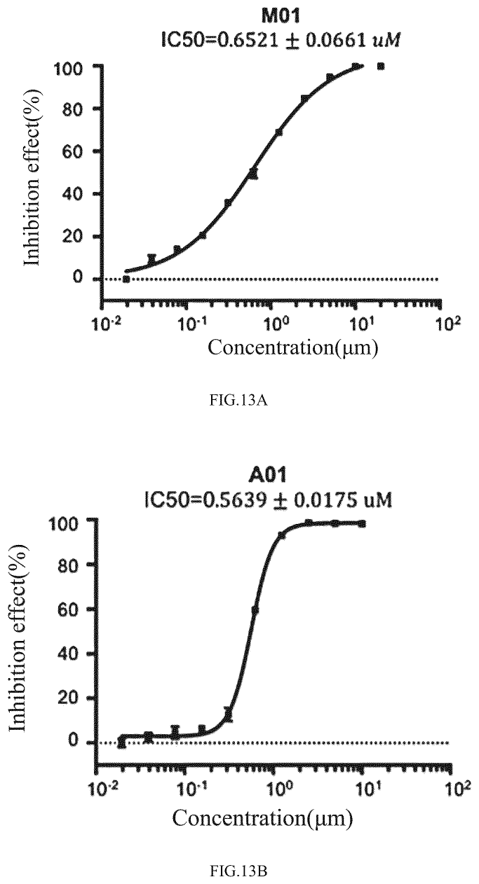

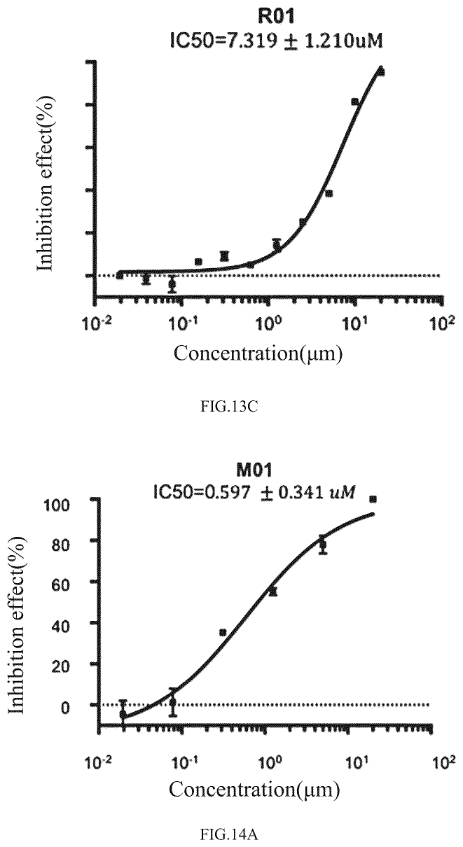

HEK293T cells in a good growth state were evenly plated in a 96-well culture plate treated with polylysine (at a cell density of about 6.5×10⁴/cm²), and the cells were uniformly distributed individually. After about 24 h of culture, the cell confluence was close to 80%. According to the steps in Example 2, transfection was carried out with ps2V, ps2AN, ps2AC, and ps2B plasmids in proportion. 6 h after transfection, each well was charged with a drug from a library of proprietary drugs. 24 h after drug treatment, the cellar luciferase activity was detected, and the inhibition rate was calculated with DMSO control as a baseline. After four rounds of screening, it was preliminarily determined that the drugs M01, A01, and R01 had inhibitory effects on viral RNA replication, and the IC50 of the drug was calculated using Graphpad Prism 7.0 software. The specific results were shown in FIG. 13A, FIG. 13B and FIG. 13C, and it could be seen that the IC50 of M01 was 0.6521±0.0661 μM, the IC50 of A01 was 0.5639±0.0175 μM, and the IC50 of R01 was 7.319±1.210 μM.

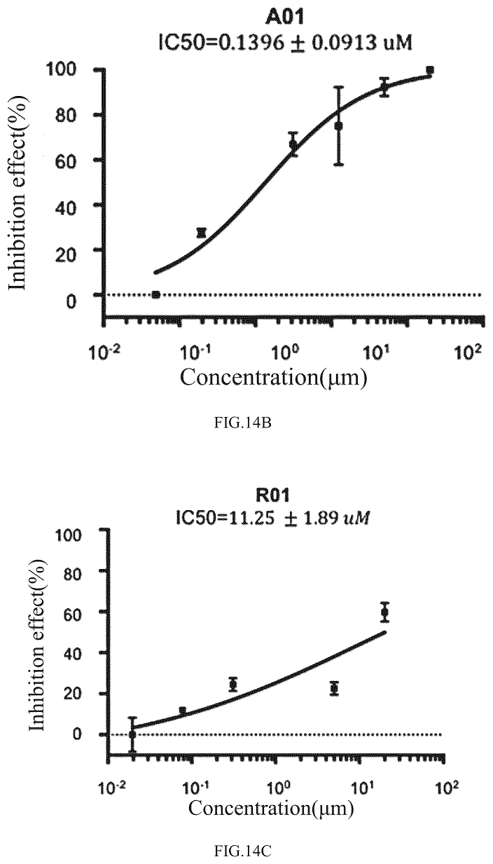

Subsequently, the inhibitory effects of the candidate drugs M01, A01, and R01 on wild-type novel coronavirus SARS-COV-2 were further verified. HEK293T cells in a good growth state were evenly plated in a 48-well culture plate treated with polylysine (at a cell density of about 6.5×10⁴/cm²). After 16 h of cell growth (the cell density was about 1.6×10⁵/mL), the cells were transfected with 0.1 g of the plasmid pCMV-ACE2-FLAG expressing SARS-COV-2 binding receptor ACE2 gene. 24 h after transfection, the cells were rinsed with PBS and infected with wild-type novel coronavirus SARS-COV-2 (MOI=0.1, 37° C., 1 h). Subsequently, DMEM (2% FBS) comprising the drugs M01, A01, and R01 with different concentration gradients (20) μM, 5 μM, 1.25 μM, 0.3125 μM, 0.078125 μM, and 0.01953125 μM) was replaced. After 24 h of drug treatment, cell RNA was extracted by TRIZOL, and RNA copies of SARS-COV-2 were detected by means of novel coronavirus 2019-nCOV nucleic acid detection (PCR-fluorescence probe method) from Daan Gene. The Ct value was obtained, the virus copy number was calculated based on a standard curve, the inhibition rate was calculated, and the IC50 of the drug was calculated using Graphpad Prism 7.0 software. The results were shown in FIG. 14A, FIG. 14B, and FIG. 14C.

It could be seen that when inhibiting the growth of wild-type SARS-COV-2, the IC50 of M01 was 0.597±0.341 μM, the IC50 of A01 was 0.1396±0.0913 μM, and the IC50 of R01 was 11.25±1.89 μM, showing obvious resistance.

The above experimental results further indicated that the candidate drugs screened by the SARS-COV-2 replicon system constructed in Example 1 could effectively inhibit wild-type SARS-COV-2, and the SARS-COV-2 replicon system could be used as a reliable anti-SARS-CoV-2 drug screening system.

### Example 5 Detection and Evaluation of the Effect of Mutation on Viral Replication by Using Novel Coronavirus SARS-COV-2 Replicon System

According to the results of the above-mentioned examples, it could also be expected that the replicon system constructed in Example 1 could be used to monitor the effect of a mutation produced in SARS-COV-2 during an epidemic on SARS-COV-2 virus replication.

Study on viral molecule evolution was shown in FIG. 15. SARS-COV-2 was prevalent worldwide. 5′ UTR_241 C was the dominant strain in the early epidemic of the virus, and 5′ UTR_241 T was currently the main strain prevalent (as of August 2020).

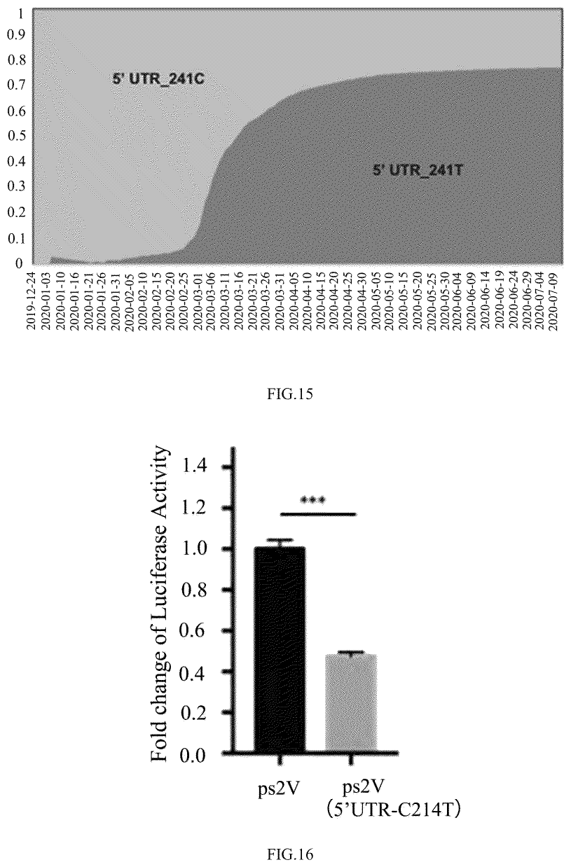

In the replicon system constructed in Example 1, 5′ UTR was located on the ps2V molecule. C at position 241 in 5′ UTR of ps2V was mutated into T by using Mut Express II Fast Mutagenesis kit from Vazyme to construct 5′ UTR_241 T_ps2V. Transfection with 5′ UTR_241 T_ps2V was carried out according to the experimental method of Example 2, and 5′ UTR_241 C_ps2V was used as an experimental control. The intracellular luciferase activity was detected by luciferase detection system, and the results were shown in FIG. 16.

As could be seen from FIG. 16, the luciferase activity of 5′ UTR_241 T_ps2V was lower than that of 5′ UTR_241 C_ps2V, indicating that the mutation in 5′ UTR_C24IT had a negative effect on virus replication, which indicated to a certain extent that the virulence of the currently prevalent 5′ UTR_241 T strain was reduced than that of the earlier prevalent 5′ UTR_241 C strain.

The above examples only express several embodiments of the present disclosure, and the description thereof is relatively specific and detailed, but they cannot be understood as a limitation on the scope of protection for the present disclosure. It should be pointed out that for those of ordinary skill in the art, a number of modifications and improvements could also be made without departing from the concept of the present disclosure, and these all fall within the scope of protection of the present disclosure.

1. A novel coronavirus SARS-COV-2 replicon structure, comprising the following nucleic acid sequences:
(I) a nucleic acid sequence encoding a novel coronavirus SARS-COV-2 non-structural protein; and
(II) nucleic acid sequences of 5′ UTR and 3′ UTR of the novel coronavirus SARS-COV-2, a transcription regulatory region on which the novel coronavirus SARS-COV-2 non-structural protein can act, and a reporter gene.

2. The replicon structure according to claim 1, wherein the non-structural protein is at least one of novel coronavirus SARS-COV-2 protein nsps 1-16.

3. The replicon structure according to claim 1, wherein the transcription regulatory region is at least one of transcription regulatory regions of S, ORF3a, M, ORF7a, ORF8 or N genes of novel coronavirus SARS-COV-2.

4. The replicon structure according to claim 1, wherein the transcription regulatory region is located upstream of the reporter gene.

5. The replicon structure according to claim 1, further comprising a nucleic acid sequence of an additional reporter gene as a reference.

6. The replicon structure according to claim 5, wherein the additional reporter gene as the reference is connected to a stop codon and located upstream of the transcription regulatory region.

7. The replicon structure according to claim 1, wherein the nucleic acid is DNA or RNA, preferably antisense RNA.

8. A novel coronavirus SARS-COV-2 replicon system, comprising an expression vector in which the replicon structure according to claim 1 is inserted.

9. The replicon system according to claim 8, comprising the following two expression vectors comprising:
(i) a nucleic acid sequence encoding a novel coronavirus SARS-COV-2 non-structural protein; and
(ii) nucleic acid sequences of 5′ UTR and 3′ UTR of a novel coronavirus SARS-COV-2, a transcription regulatory region on which the novel coronavirus SARS-COV-2 non-structural protein can act, and a reporter gene.

10. The replicon system according to claim 9, wherein in expression vector (ii), nucleic acid sequences of the 5′ UTR of novel coronavirus SARS-COV-2, the transcription regulatory region on which the novel coronavirus SARS-COV-2 non-structural protein can act, the reporter gene, and the 3′ UTR of novel coronavirus SARS-COV-2 are inserted in order.

11. The replicon system according to claim 9, wherein in expression vector (ii), nucleic acid sequences of the 5′ UTR of novel coronavirus SARS-COV-2, a reporter gene A, the transcription regulatory region on which the novel coronavirus SARS-COV-2 non-structural protein can act, a reporter gene B, and the 3′ UTR of novel coronavirus SARS-COV-2 are inserted in order, wherein the reporter gene A is different from the reporter gene B.

12. The replicon system according to claim 11, wherein a nucleic acid sequence of a ribosome entry site is further connected between the 5′ UTR of novel coronavirus SARS-COV-2 and the reporter gene A.

13. The replicon system according to claim 11, wherein the reporter gene A is a nucleic acid sequence of fluorescent protein; and the reporter gene B is a nucleic acid sequence encoding luciferase.

14. The replicon system according to claim 11, wherein the nucleic acid sequence inserted in expression vector (ii) is shown in SEQ ID NO: 28.

15. The replicon system according to claim 9, wherein the encoded novel coronavirus SARS-COV-2 non-structural protein is novel coronavirus SARS-COV-2 protein nsps 1-16.

16. The replicon system according to claim 15, wherein expression vector (i) comprises three expression vectors, in which nucleic acid sequences encoding one or more of novel coronavirus SARS-COV-2 protein nsps 1-16 are respectively inserted.

17. The replicon system according to claim 16, wherein a nucleic acid sequence encoding novel coronavirus SARS-COV-2 protein nsps 1-4, a nucleic acid sequence encoding novel coronavirus SARS-COV-2 protein nsps 5-11, and a nucleic acid sequence encoding novel coronavirus SARS-COV-2 protein nsps 12-16 are respectively inserted in the three expression vectors.

18. The replicon system according to claim 16, wherein the nucleic acid sequences respectively inserted in the three expression vectors are shown in SEQ ID NOs: 17-19.

19. A packaging cell, comprising the replicon structure according to claim 1.

20. The packaging cell according to claim 19, wherein the cell is a human-derived cell.

21. The packaging cell according to claim 20, wherein the replicon structure or replicon system is codon-optimized.

22. (canceled)

23. A method for screening an anti-novel coronavirus SARS-COV-2 drug, comprising adding a drug to be tested to an expression system comprising the replicon structure according to claim 1 to detect the differential expression of a reporter gene and evaluate an anti-novel coronavirus SARS-COV-2 effect of the drug to be tested.

24. A kit for screening an anti-novel coronavirus SARS-COV-2 drug, comprising the replicon structure according to claim 1.

25. A system for screening an anti-novel coronavirus SARS-COV-2 drug, comprising the replicon structure according to claim 1.

26. The drug screening system according to claim 25, further comprising a luciferase detection device, preferably a fluorescent protein detection device, and more preferably a fully automatic robotic arm drug screening platform.

27. A novel coronavirus SARS-COV-2 molecular epidemiological monitoring system, comprising the replicon structure according to claim 1.

28. The novel coronavirus SARS-COV-2 molecular epidemiological monitoring system according to claim 27, wherein the replicon system is used to monitor an effect of a mutation produced in SARS-COV-2 during an epidemic on SARS-COV-2 virus replication.

29. A packaging cell, comprising the replicon system according to claim 8.

30. A method for screening an anti-novel coronavirus SARS-COV-2 drug, comprising adding a drug to be tested to an expression system comprising the replicon system according to claim 8 to detect the differential expression of a reporter gene and evaluate an anti-novel coronavirus SARS-COV-2 effect of the drug to be tested.

31. A method for screening an anti-novel coronavirus SARS-COV-2 drug, comprising adding a drug to be tested to an expression system comprising the packaging cell according to claim 19 to detect the differential expression of a reporter gene and evaluate an anti-novel coronavirus SARS-COV-2 effect of the drug to be tested.

32. A kit for screening an anti-novel coronavirus SARS-COV-2 drug, comprising the replicon system according to claim 8.

33. A kit for screening an anti-novel coronavirus SARS-COV-2 drug, comprising the packaging cell according to claim 19.

34. A system for screening an anti-novel coronavirus SARS-COV-2 drug, comprising the replicon system according to claim 8.

35. A system for screening an anti-novel coronavirus SARS-COV-2 drug, comprising the packaging cell according to claim 19.

36. A novel coronavirus SARS-COV-2 molecular epidemiological monitoring system, comprising the replicon system according to claim 8.

37. A novel coronavirus SARS-COV-2 molecular epidemiological monitoring system, comprising the packaging cell according to claim 19.


SEQUENCE LISTING (30 sequences)

SEQ ID NO: 1 - DNA, 540 bp, Artificial Sequence
  synthesized
atggagtccctggtgcccggcttcaacgagaagacccacgtgcagctgtctctgcctgtgctgcaggtgagggatgtgctggtgcgcggctttggcgactccgtcgaggaggtgctgtctgaggccaggcagcacctgaaggacggaacctgcggactggtggaggtggagaagggcgtgctgccacagctggagcagccttacgtgttcatcaagaggtccgatgcaaggacagcaccacacggacacgtgatggtggagctggtggccgagctggagggcatccagtatggccgctctggagagaccctgggcgtgctggtgccacacgtgggagagatcccagtggcctatcggaaggtgctgctgagaaagaacggcaataagggagcaggaggacactcttacggagcagacctgaagagcttcgatctgggcgacgagctgggcaccgatccttatgaggactttcaggagaactggaatacaaagcacagctccggcgtgacccgggagctgatgagagagctgaacggcggc

SEQ ID NO: 2 - DNA, 1914 bp, Artificial Sequence
  synthesized
gcctacaccagatatgtggataacaatttctgcggaccagacggataccccctggagtgtatcaaggatctgctggccagagcaggcaaggcctcctgcaccctgtctgagcagctggacttcatcgacacaaagcggggcgtgtattgctgtagagagcacgagcacgagatcgcctggtataccgagcggtccgagaagtcttacgagctgcagacaccattcgagatcaagctggccaagaagttcgacaccttcaacggcgagtgtccaaacttcgtgtttcccctgaatagcatcatcaagaccatccagcccagagtggagaagaagaagctggatggctttatgggcaggatccgcagcgtgtaccctgtggcctccccaaacgagtgcaatcagatgtgcctgtccacactgatgaagtgcgatcactgtggcgagacctcttggcagacaggcgacttcgtgaaggccacctgcgagttttgtggcaccgagaacctgacaaaggagggcgccaccacatgcggctatctgcctcagaatgccgtggtgaagatctactgcccagcctgtcacaactccgaagtgggaccagagcactctctggccgagtaccacaatgagtccggcctgaagacaatcctgaggaagggaggaaggaccatcgccttcggcggatgcgtgttttcttatgtgggctgccacaacaagtgtgcatactgggtgccaagggccagcgccaatatcggctgtaaccacaccggagtggtgggagagggatccgagggcctgaacgataatctgctggagatcctgcagaaggagaaggtgaacatcaatatcgtgggcgacttcaagctgaacgaggagatcgccatcatcctggcctccttctctgccagcacatccgcctttgtggagaccgtgaagggcctggactacaaggccttcaagcagatcgtggagagctgcggcaacttcaaggtgaccaagggcaaggccaagaagggcgcctggaacatcggcgagcagaagagcatcctgtcccctctgtatgccttcgccagcgaggcagcaagggtggtgagatctatctttagccggaccctggagacagcccagaattccgtgagagtgctgcagaaggccgccatcaccatcctggatggcatctcccagtactctctgaggctgatcgatgccatgatgttcacctccgacctggccacaaacaatctggtggtcatggcctacatcaccggcggcgtggtgcagctgacctctcagtggctgacaaacatctttggcaccgtgtatgagaagctgaagccagtgctggattggctggaggagaagttcaaggagggcgtggagtttctgcgcgacggctgggagatcgtgaagttcatcagcacctgcgcatgtgagatcgtgggaggacagatcgtgacctgtgccaaggagatcaaggagtccgtgcagacattctttaagctggtgaacaagttcctggccctgtgcgccgactctatcatcatcggcggcgccaagctgaaggccctgaacctgggcgagacctttgtgacacacagcaagggcctgtacaggaagtgcgtgaagtcccgcgaggagaccggactgctgatgcccctgaaggcacctaaggagatcatcttcctggagggcgagaccctgcccacagaggtgctgacagaggaggtggtgctgaagaccggcgacctgcagccactggagcagcccaccagcgaggcagtggaggcacctctggtgggcacaccagtgtgcatcaatggcctgatgctgctggagatcaaggataccgagaagtactgtgccctggcccctaacatgatggtgacaaacaataccttcacactgaagggcggc

SEQ ID NO: 3 - DNA, 5949 bp, Artificial Sequence
  synthesized
gccccaaccaaggtgacatttggcgacgataccgtgatcgaggtgcagggctacaagtctgtgaatatcacattcgagctggatgagagaatcgacaaggtgctgaacgagaagtgcagcgcctatacagtggagctgggcaccgaggtgaacgagtttgcctgcgtggtggccgacgccgtgatcaagaccctgcagccagtgtccgagctgctgacacccctgggcatcgatctggacgagtggtctatggccacctactatctgttcgacgagagcggcgagtttaagctggcctcccacatgtactgctctttctatccccctgatgaagacgaggaggagggcgattgcgaggaggaggagtttgagcccagcacacagtacgagtatggcaccgaggacgattaccagggcaagccactggagttcggagccacctccgccgccctgcagccagaggaggagcaggaggaggattggctggacgatgactcccagcagaccgtgggccagcaggatggctctgaggacaatcagaccacaaccatccagacaatcgtggaggtgcagcctcagctggagatggagctgaccccagtggtgcagaccatcgaggtgaactctttcagcggctatctgaagctgacagataacgtgtacatcaagaacgccgacattgtggaggaggccaagaaggtgaagcctaccgtggtggtgaacgccgccaacgtgtacctgaagcacggaggaggagtggcaggcgccctgaacaaggccaccaacaatgccatgcaggtggagagcgatgactatatcgccacaaatggacccctgaaggtcggaggaagctgcgtgctgtccggacacaacctggccaagcactgtctgcacgtggtgggccctaacgtgaataagggcgaggacatccagctgctgaagtccgcctacgagaacttcaatcagcacgaggtgctgctggcccctctgctgagcgccggcatctttggcgccgatccaatccactccctgagggtgtgcgtggacaccgtgcgcacaaacgtgtacctggccgtgttcgataagaacctgtacgacaagctggtgtctagctttctggagatgaagagcgagaagcaggtggagcagaagatcgccgagatccctaaggaggaggtgaagccattcatcaccgagagcaagccttccgtggagcagaggaagcaggatgacaagaagatcaaggcctgcgtggaggaggtgacaaccacactggaggagaccaagttcctgacagagaacctgctgctgtacatcgatatcaacggcaatctgcacccagacagcgccacactggtgtccgatatcgacatcacctttctgaagaaggatgccccatatatcgtgggcgacgtggtgcaggagggcgtgctgacagccgtggtcatccccaccaagaaggccggcggcaccacagagatgctggccaaggccctgcgcaaggtgcctaccgacaattacatcaccacatatccaggccagggcctgaacggctataccgtggaggaggccaagaccgtgctgaagaagtgcaagagcgccttctacatcctgccttctatcatcagcaatgagaagcaggagatcctgggcaccgtgtcctggaacctgagggagatgctggcccacgccgaggagacacgcaagctgatgcccgtgtgcgtggagacaaaggccatcgtgagcaccatccagcggaagtataagggcatcaagatccaggagggagtggtggactacggagcaagattctacttttatacctctaagaccacagtggccagcctgatcaacacactgaatgatctgaacgagaccctggtgacaatgcccctgggctatgtgacccacggcctgaatctggaggaggccgccaggtacatgcgctccctgaaggtgccagcaaccgtgagcgtgagctctcctgacgccgtgacagcctacaacggctatctgacaagctcctctaagaccccagaggagcacttcatcgagaccatctctctggccggcagctataaggattggtcctactctggccagtccacacagctgggcatcgagtttctgaagaggggcgacaagagcgtgtactataccagcaatcccaccacattccacctggatggcgaagtgatcaccttcgacaacctgaagaccctgctgagcctgcgggaggtgagaaccatcaaggtgttcaccacagtggataacatcaatctgcacacacaggtggtggacatgtccatgacctatggccagcagtttggcccaacatacctggatggcgccgacgtgaccaagatcaagccccacaatagccacgagggcaagacattctacgtgctgcctaatgccaccaacttttccctgctgaagcaggcaggcgacgtggaggagaacccaggaccagatgacaccctgagggtggaggccttcgagtactatcacaccacagatcctagctttctgggccgctatatgtccgccctgaatcacaccaagaagtggaagtacccacaggtgaacggcctgacaagcatcaagtgggccgacaacaattgctacctggccaccgccctgctgacactgcagcagatcgagctgaagttcaacccacccgccctgcaggatgcatactatagggcaagagcaggagaggcagccaatttttgcgccctgatcctggcctattgtaacaagaccgtgggagagctgggcgatgtgcgggagacaatgagctacctgttccagcacgccaatctggactcctgcaagagagtgctgaacgtggtgtgcaagacatgtggccagcagcagaccacactgaagggcgtggaggccgtgatgtatatgggcaccctgagctacgagcagtttaagaagggcgtgcagatcccctgcacatgtggcaagcaggccaccaagtacctggtgcagcaggagtcccctttcgtgatgatgtctgcccctccagcccagtatgagctgaagcacggcacctttacatgcgcctctgagtacaccggcaattatcagtgtggccactataagcacatcaccagcaaggagacactgtactgcatcgatggcgccctgctgaccaagagctccgagtacaagggccccatcacagacgtgttctataaggagaattcttacaccacaaccatcgccaccaactttagcctgctgaagcaggccggcgatgtggaggagaaccctggaccaaagcccgtgacctataagctggacggcgtggtgtgcacagagatcgatcctaagctggacaactactacaagaaggataactcttatttcaccgagcagcccatcgacctggtgcctaatcagccttacccaaacgccagcttcgataatttcaagttcgtgtgcgacaatatcaagtttgccgatgacctgaaccagctgaccggatacaagaagccagccagccgggagctgaaggtgacattctttcctgatctgaacggcgacgtggtggccatcgactacaagcactatacaccttccttcaagaagggcgccaagctgctgcacaagccaatcgtgtggcacgtgaacaatgccaccaataaggccacatacaagccaaacacctggtgcatcagatgtctgtggtctacaaagcccgtggagaccagcaattcctttgatgtgctgaagagcgaggatgcccagggcatggacaacctggcctgcgaggacctgaagcccgtgagcgaggaggtggtggagaatcctaccatccagaaggatgtgctggagtgtaacgtgaagacaaccgaggtggtgggcgacatcatcctgaagcctgccaacaattccctgaagatcacagaggaagtgggccacaccgatctgatggccgcctacgtggacaattctagcctgaccatcaagaagccaaacgagctgagcagggtgctgggcctgaagaccctggccacacacggcctggccgcagtgaattccgtgccatgggacaccatcgccaattatgccaagcccttcctgaacaaggtggtgagcacaaccacaaacatcgtgacacggtgcctgaaccgggtgtgcaccaattacatgccatatttctttacactgctgctgcagctgtgcacctttacaaggtccaccaattctcgcatcaaggcctccatgcccaccacaatcgccaagaacacagtgaagagcgtgggcaagttctgcctggaggcctcctttaactacctgaagtcccccaatttctctaagctgatcaacatcatcatctggtttctgctgctgagcgtgtgcctgggcagcctgatctattccacagccgccctgggcgtgctgatgagcaacctgggcatgccttcctactgcaccggctatcgggagggctacctgaatagcaccaacgtgacaatcgccacctactgtacaggctctatcccatgcagcgtgtgcctgtccggcctggattctctggacacctatccttccctggagaccatccagatcacaatctcctctttcaagtgggacctgaccgcctttggcctggtggcagagtggttcctggcctatatcctgtttacaagattcttttacgtgctgggcctggccgccatcatgcagctgttctttagctacttcgccgtgcactttatctctaatagctggctgatgtggctgatcatcaacctggtgcagatggcccccatctccgccatggtgaggatgtatatcttctttgcctctttctactacgtgtggaagagctacgtgcacgtggtggacggctgcaatagctccacctgcatgatgtgctacaagaggaaccgcgccacacgcgtggagtgtaccacaatcgtgaatggcgtgcggagaagcttctacgtgtatgccaacggcggcaagggcttttgcaagctgcacaactggaattgcgtgaactgtgatacattctgtgccggcagcacctttatctccgatgaggtggcaagggacctgtccctgcagttcaagagaccaatcaatcccaccgatcagtctagctacatcgtggactccgtgacagtgaagaacggctctatccacctgtatttcgataaggccggccagaagacatacgagaggcactccctgtctcactttgtgaatctggacaacctgcgcgccaacaataccaagggcagcctgcccatcaacgtgatcgtgttcgatggcaagtccaagtgcgaggagtcctctgccaagagcgcctccgtgtactatagccagctgatgtgccagcctatcctgctgctggaccaggccctggtgtccgatgtgggcgactctgccgaggtggcagtgaagatgtttgatgcctacgtgaataccttcagcagcaccttcaacgtgccaatggagaagctgaagaccctggtggcaacagcagaggcagagctggccaagaacgtgtccctggacaatgtgctgtctaccttcatcagcgccgcccgccagggctttgtggattctgacgtggagacaaaggatgtggtggagtgcctgaagctgagccaccagtccgatatcgaggtgaccggcgacagctgtaacaattatatgctgacctacaataaggtggagaacatgacaccccgggatctgggcgcctgcatcgactgttctgccagacacatcaatgcccaggtggccaagagccacaatatcgccctgatctggaacgtgaaggacttcatgtctctgagcgagcagctgaggaagcagatccgctccgccgccaagaagaacaatctgcccttcaagctgacctgcgccaccacaaggcaggtggtgaacgtggtcaccacaaagatcgccctgaagggcggc

SEQ ID NO: 4 - DNA, 1503 bp, Artificial Sequence
  synthesized
aagatcgtgaacaattggctgaagcagctgatcaaggtgaccctggtgttcctgtttgtggccgccatcttctacctgatcacccccgtgcacgtgatgtctaagcacacagatttttctagcgagatcatcggctataaggccatcgacggaggagtgaccagggatatcgccagcaccgacacatgcttcgccaataagcacgccgatttcgacacctggtttagccagaggggcggctcctacacaaacgacaaggcctgtccactgatcgcagccgtgatcaccagggaagtgggattcgtggtgcctggactgccaggaacaatcctgaggaccacaaatggcgacttcctgcactttctgcctcgcgtgttttccgccgtgggcaacatctgctataccccatctaagctgatcgagtacaccgatttcgccacatccgcctgcgtgctggccgcagagtgtaccatctttaaggatgcctctggcaagcccgtgccttactgttatgacacaaatgtgctggagggctctgtggcctatgagagcctgcggccagataccagatacgtgctgatggacggcagcatcatccagttccccaacacatatctggagggctctgtgcgggtggtgaccacatttgacagcgagtactgccggcacggcacctgtgagagatctgaggccggcgtgtgcgtgtccacatctggcaggtgggtgctgaacaatgattactatcgcagcctgcctggcgtgttctgtggcgtggacgccgtgaatctgctgaccaacatgtttacacctctgatccagccaatcggcgccctggatatcagcgcctccatcgtggcaggaggaatcgtggcaatcgtggtgacatgcctggcctactatttcatgcggttccggagggccttcggcgagtactctcacgtggtggcctttaataccctgctgttcctgatgagcttcaccgtgctgtgcctgacccccgtgtatagcttcctgcctggcgtgtactccgtgatctacctgtatctgaccttctacctgacaaacgacgtgagctttctggcccacatccagtggatggtcatgttcacccccctggtgcctttttggatcacaatcgcctatatcatctgcatctccaccaagcacttctattggttcttttctaattacctgaagcggagagtggtgtttaacggcgtgtctttcagcacctttgaggaggccgccctgtgcacattcctgctgaacaaggagatgtacctgaagctgcggtccgacgtgctgctgccactgacccagtacaatagatatctggccctgtataacaagtacaagtatttctctggcgccatggataccacaagctacagagaggcagcatgctgtcacctggcaaaggccctgaatgatttttccaactctggcagcgacgtgctgtaccagccccctcagacctctatcacaagcgccgtgctgcagtaa

SEQ ID NO: 5 - DNA, 918 bp, Artificial Sequence
  synthesized
agtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaa

SEQ ID NO: 6 - DNA, 870 bp, Artificial Sequence
  synthesized
agtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacag

SEQ ID NO: 7 - DNA, 249 bp, Artificial Sequence
  synthesized
tctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaa

SEQ ID NO: 8 - DNA, 594 bp, Artificial Sequence
  synthesized
gctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacag

SEQ ID NO: 9 - DNA, 339 bp, Artificial Sequence
  synthesized
aataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaa

SEQ ID NO: 10 - DNA, 417 bp, Artificial Sequence
  synthesized
gctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcag

SEQ ID NO: 11 - DNA, 39 bp, Artificial Sequence
  synthesized
tcagctgatgcacaatcgtttttaaacgggtttgcggtg

SEQ ID NO: 12 - DNA, 2799 bp, Artificial Sequence
  synthesized
atgtcagcagatgcacaatcatttcttaacagagtgtgcggagtgtcagcagcaagacttacaccttgcggaacaggaacatcaacagatgtagtttatagggccttcgatatctacaacgataaagtggcaggatttgcaaagttcttaaagaccaattgctgcagatttcaagagaaggacgaggatgataaccttatcgattcatactttgtggtgaagaggcatacattcagcaattaccaacacgaagaaacaatctacaaccttcttaaagattgccctgcagtggcaaagcatgacttcttcaagttcagaatcgatggagatatggtgcctcacatctcaagacaaagacttacaaagtatacgatggcagatctcgtttatgcgttgcgccatttcgacgagggtaattgtgacaccctgaaggagatcctggtcacgtataattgctgcgatgatgattactttaacaagaaggactggtatgatttcgtagagaatcctgacattcttagagtgtacgcaaaccttggagaaagagtgagacaagcactcctaaagacagttcaattctgcgacgcaatgagaaacgcaggaatcgtgggagtgcttacacttgataaccaagatcttaacggaaactggtatgactttggcgactttatacagacaacacctggatcaggagtgcctgtggtggattcatattatagcctgctgatgcctatccttacacttacaagagcacttacagcagaatcacatgtggataccgacttgaccaaaccctatattaaatgggatctgctgaaatatgactttacagaagaacgacttaaactcttcgacagatactttaaatactgggatcaaacataccaccctaactgcgtgaactgccttgatgatagatgcatccttcactgcgcaaactttaacgtgctgttctcgaccgtgtttcctcctacatcatttggacctcttgtgagaaagatctttgtggacggagtacctttcgtcgtatcaacaggataccactttagagaacttggagtagtgcataatcaagatgtgaacctacattctagccgattatcatttaaagaacttctggtttatgccgcggaccctgcaatgcacgcagcaagtggcaatttattacttgacaaacggacaacctgtttctcggttgccgcacttacaaacaatgtagctttccagaccgtaaagccagggaatttcaacaaagatttctatgacttcgccgtatcaaagggattcttcaaggagggatcatcagtggaacttaaacacttcttcttcgcccaggatggaaacgcagcaatctcagattacgattactacagatacaaccttcctacaatgtgcgatatcagacaacttctcttcgtagttgaagtggtggataaatactttgattgctacgatggaggatgcatcaacgcaaaccaagtgatcgtgaacaacttggataaatccgctggattcccgtttaataagtggggtaaagcccgcctttactacgattcaatgtcatacgaagatcaagatgcattattcgcttatacaaagaggaatgtgatccctacaatcacacaaatgaaccttaaatacgcaatctcagcaaagaatcgagcaagaacagtggcaggagtgtcaatctgctcaacaatgacaaacagacaatttcaccagaagctcctgaaatcaatcgcagcaacaagaggagcaacagtggtgatcggaacatcaaagttctatggaggttggcacaacatgctcaagaccgtgtatagcgatgttgagaatccgcatctcatgggatgggattaccctaaatgcgatagagctatgcccaatatgctgagaatcatggcatcacttgtgcttgcaagaaagcataccacatgctgctcactttcacacagattctatcgacttgcaaacgaatgcgcacaggtcctctccgagatggtgatgtgcggcgggagcttgtatgtgaaaccaggtggaacatcatcaggagatgcaacaacagcatacgcaaactcagtgtttaacatctgccaagcagtgacagctaatgtaaacgctctcttgagcactgacggaaacaagatagccgataaatacgtgcgtaatctgcagcatcgactttacgaatgcctttacagaaacagagatgtagacacggactttgtaaatgaattctatgcttaccttagaaagcatttctccatgatgatactgagtgacgatgctgttgtatgtttcaactcaacatacgcatcacaaggacttgtggcatcaatcaagaatttcaaatcagtgctttactaccagaataatgtgtttatgtcagaagcaaagtgttggacagaaactgacctcactaagggccctcacgagttctgtagccaacacacaatgcttgtgaaacaaggagatgactatgtttatctcccataccctgatccttcaagaatcttgggtgcagggtgtttcgtggatgatatcgtgaagactgacggaacacttatgatcgaaagatttgtgtcacttgcaatcgatgcataccctcttacaaagcatccgaaccaagaatacgcagatgtgtttcacctttaccttcaatacatcagaaagttgcatgatgaacttacaggacacatgcttgatatgtactcagtgatgcttacaaacgataacacatcaagatactgggaacctgaattctatgaggcaatgtacacacctcacacagtgcttcaa

SEQ ID NO: 13 - DNA, 1803 bp, Artificial Sequence
  synthesized
gcagtgggagcatgcgtgctttgcaactcacaaacatcacttagatgcggagcatgcatcagaagacctttcctgtgttgcaaatgctgctacgatcacgtgatctcaacatcacacaaacttgtgctttcagtgaacccttacgtgtgcaacgcaccaggctgtgacgtaactgacgttacgcagctctatcttggaggaatgtcatactactgcaaatcacacaaacctcctatctcatttcctctttgcgcaaacggacaagtgtttggactttacaagaatacttgcgtgggatcagataacgtgacagatttcaatgctatcgcaacatgcgattggacaaacgcaggagattacatccttgcaaacacatgcacagagcgtctgaagttgtttgcggccgaaacacttaaagcaacagaagaaacatttaaactttcatacggaatcgcaacagtgagagaggtcctatcggacagggaactccacctttcatgggaagtgggcaaaccacgcccgccgcttaacagaaactacgtgtttacaggatacagagtgacaaagaattctaaggtacagatcggagaatacacatttgagaagggcgactacggagacgccgtggtgtacagagggacgactacgtataaacttaacgtgggagattactttgtgcttacatcacacacagtgatgcctctttcagcacctacacttgtgcctcaagagcattatgtccgaataacgggtctctatccgacacttaacatctcagatgaattctcgagtaacgtggcaaactaccagaaagtgggtatgcagaaatactccaccttacagggacctcctggtacaggaaagtctcatttcgcgataggtctagctctctattacccttcagcaagaatcgtgtacacagcatgctcacacgcagcagtggatgcactttgcgagaaggcgctgaaataccttcctatcgataaatgctcaagaatcatccctgcaagagcaagagtggaatgctttgataaatttaaagtgaactcaacacttgaacaatacgtgttctgtactgtaaatgctctgcctgaaactaccgcggatatcgtggtgttcgacgagatatccatggcaacaaactacgacctatcggtcgtaaacgcgcggctaagagcaaagcattatgtgtacatcggagatcctgcacaacttcctgcacctagaacattactaactaaagggacgctcgaacctgaatactttaacagtgtttgtcgcctaatgaagacgatcgggccggacatgtttcttggaacatgcagaagatgccctgcagaaatcgtggatacagtgtcagcacttgtgtacgataacaaacttaaagcacacaaagacaagtcggctcagtgtttcaagatgttttacaaaggagtgatcacacacgatgtgtcatcagcaatcaacagacctcaaatcggagtggtgagagaatttcttacaagaaaccctgcatggagaaaggcggtcttcataagtccttacaactcacagaatgccgtggcatcaaagatactcgggcttcctacacaaacagtggattcatcacaaggatcagaatacgattacgtgatctttacacaaacaacagaaacagcacactcatgcaacgtgaacagatttaacgtggcaatcacaagagcaaaggtagggatcctctgtatcatgtcagatagagatctttacgataaacttcaatttacatcacttgaaatccctagaagaaacgtggcgactctgcag

SEQ ID NO: 14 - DNA, 1581 bp, Artificial Sequence
  synthesized
gctgagaacgtgacaggattgttcaaggactgctcaaaggtaattacgggtttacatccgacacaagcacctacacacctttcagtggatacaaagttcaagactgaaggactttgcgtggatatccctggaatccctaaagatatgacatacagaagacttatctcaatgatgggatttaagatgaattaccaagtgaacggataccctaacatgtttatcacaagagaagaagcaatcagacacgtgagagcatggataggcttcgacgtcgagggatgccacgcaacaagagaagcagtgggaacaaaccttcctcttcaacttggattctccactggagtgaaccttgtggcagtgcctacaggatacgtggatacacctaacaacacagatttctcgcgagtgtcagcaaagccaccacctggagatcaatttaaacaccttatccctcttatgtacaaaggacttccttggaacgtggtgagaatcaagatagtccaaatgctatccgataccttaaagaatcttagtgaccgtgtcgtatttgtgctttgggcacacggatttgaacttacatcaatgaaatactttgtgaagatcggtcccgagcgtacatgctgcctttgcgatagaagagctacgtgtttcagtaccgcttcagatacatacgcatgctggcaccactcaataggcttcgattacgtttataatccgttcatgatagatgtgcaacaatggggattcacgggcaatctgcagagcaaccacgatctttactgccaagtgcacggaaacgcacacgtggcatcatgcgatgcaatcatgacaagatgccttgcagtgcacgaatgctttgtgaagcgggtcgattggacaatcgaataccctatcatcggagatgaacttaagataaatgcagcatgcagaaaggtccagcacatggtggtgaaagcagcacttcttgcagataaatttcctgtgcttcacgatatcggaaaccctaaagcaatcaaatgcgtgcctcaagcagatgtggaatggaaattctatgacgcacaaccttgctcagataaagcatacaagatagaggaactattctatagttacgcaacacactcagataaatttacagatggagtgtgcctgttctggaattgcaacgtggatagataccctgcaaactcaatcgtgtgcagatttgatacaagagtgctttcaaaccttaaccttccaggttgtgacggcggcagtctatatgttaataagcacgcatttcacacacctgcattcgataagtccgcattcgtcaatttaaagcagctacctttcttctattattcagattcaccttgcgaatcacacggaaagcaggttgtcagtgacatcgattacgtgcctcttaaatcagcaacatgtattaccaggtgtaatcttggaggagccgtctgtcgacatcatgcaaacgaatacagactttaccttgatgcatacaacatgatgatctccgccgggttctccctatgggtgtacaaacaatttgatacatacaacctttggaacacatttacaagacttcaa

SEQ ID NO: 15 - DNA, 1038 bp, Artificial Sequence
  synthesized
tcacttgagaacgttgcgttcaatgtagtcaataagggacacttcgacggtcaacagggtgaggttcctgtgtcaatcatcaacaataccgtttatactaaagttgacggcgtggatgtggaactcttcgagaataagactacgcttcctgtgaatgttgccttcgagttgtgggcaaagcgcaatatcaaacctgtgcctgaagtgaagatactcaataaccttggagtggatatcgcagcaaacacagtgatctgggattacaagagggacgcacctgcacacatctcaacaatcggagtgtgctcaatgacagatatcgcaaagaagccgactgaaacaatctgcgcacctcttactgtattcttcgacggaagagtggatggacaagtggatttattccgaaatgcaagaaacggagtgcttatcacagaaggatcagtgaaaggacttcaaccttcagtgggacctaaacaagcatcacttaacggagtgactctgataggcgaggccgtgaagactcagtttaactactacaagaaagtagacggtgtcgtccagcagctgcccgagacctatttcacacaatcacggaatctgcaggagttcaaacctagatcacaaatggaaatcgatttcctggagcttgcaatggatgaatttatcgaaagatacaaacttgaaggatacgcatttgaacacatcgtgtacggagatttcagtcattcacaacttggaggacttcaccttcttattggcctagccaaacgtttcaaagaatcacctttcgagctcgaagatttcattccaatggattcaacagtgaagaattatttcattactgacgcccagacgggatcatcaaagtgtgtatgctcagtgatcgatctactactagacgatttcgttgaaattattaaatcacaagacttgagtgtagttagtaaggttgtgaaggtcacaatcgattacacagaaatctcatttatgctttggtgcaaagatggacacgtggaaacattctatcccaaacttcaa

SEQ ID NO: 16 - DNA, 897 bp, Artificial Sequence
  synthesized
tcatcacaagcatggcaacctggagtggccatgccgaatttgtataagatgcagagaatgcttcttgagaagtgtgaccttcagaattatggagattcagcaacacttcctaaaggaatcatgatgaacgtggcaaagtatactcaactttgccaataccttaacacacttacacttgcagtgccttacaacatgagagtgatccacttcggtgcagggtcggacaaaggagtggcacctggtactgctgtccttagacaatggcttcctacaggaacacttcttgtggattcagatcttaacgatttcgtctccgatgcagattcaaccctcattggtgactgtgcaacagtgcacacagcaaacaagtgggacttaataatatcagatatgtacgatcctaagactaagaatgtaacgaaagagaatgactcaaaggaaggtttcttcacctatatctgcggatttatccaacagaagttagctcttggaggatcagtggcaatcaagattacggaacactcatggaacgcagatctttacaaacttatgggacactttgcatggtggaccgcgttcgttacaaacgtaaacgcgtcgtcctcagaagcatttcttatcggatgcaactaccttgggaaaccaagagagcagatcgatggatacgtgatgcacgcaaactacatcttctggaggaacacaaaccctatccaactttcatcatactcactcttcgacatgtcaaagttcccgcttaaacttagagggactgccgtaatgtcgcttaaagaaggacaaatcaacgatatgatactcagcctcctaagtaaagggaggcttatcatcagagagaataatagagtggtgatctcatcagatgtgcttgtgaacaactaa

SEQ ID NO: 17 - DNA, 10429 bp, Artificial Sequence
  synthesized
gctagcgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatggagtccctggtgcccggcttcaacgagaagacccacgtgcagctgtctctgcctgtgctgcaggtgagggatgtgctggtgcgcggctttggcgactccgtcgaggaggtgctgtctgaggccaggcagcacctgaaggacggaacctgcggactggtggaggtggagaagggcgtgctgccacagctggagcagccttacgtgttcatcaagaggtccgatgcaaggacagcaccacacggacacgtgatggtggagctggtggccgagctggagggcatccagtatggccgctctggagagaccctgggcgtgctggtgccacacgtgggagagatcccagtggcctatcggaaggtgctgctgagaaagaacggcaataagggagcaggaggacactcttacggagcagacctgaagagcttcgatctgggcgacgagctgggcaccgatccttatgaggactttcaggagaactggaatacaaagcacagctccggcgtgacccgggagctgatgagagagctgaacggcggcgcctacaccagatatgtggataacaatttctgcggaccagacggataccccctggagtgtatcaaggatctgctggccagagcaggcaaggcctcctgcaccctgtctgagcagctggacttcatcgacacaaagcggggcgtgtattgctgtagagagcacgagcacgagatcgcctggtataccgagcggtccgagaagtcttacgagctgcagacaccattcgagatcaagctggccaagaagttcgacaccttcaacggcgagtgtccaaacttcgtgtttcccctgaatagcatcatcaagaccatccagcccagagtggagaagaagaagctggatggctttatgggcaggatccgcagcgtgtaccctgtggcctccccaaacgagtgcaatcagatgtgcctgtccacactgatgaagtgcgatcactgtggcgagacctcttggcagacaggcgacttcgtgaaggccacctgcgagttttgtggcaccgagaacctgacaaaggagggcgccaccacatgcggctatctgcctcagaatgccgtggtgaagatctactgcccagcctgtcacaactccgaagtgggaccagagcactctctggccgagtaccacaatgagtccggcctgaagacaatcctgaggaagggaggaaggaccatcgccttcggcggatgcgtgttttcttatgtgggctgccacaacaagtgtgcatactgggtgccaagggccagcgccaatatcggctgtaaccacaccggagtggtgggagagggatccgagggcctgaacgataatctgctggagatcctgcagaaggagaaggtgaacatcaatatcgtgggcgacttcaagctgaacgaggagatcgccatcatcctggcctccttctctgccagcacatccgcctttgtggagaccgtgaagggcctggactacaaggccttcaagcagatcgtggagagctgcggcaacttcaaggtgaccaagggcaaggccaagaagggcgcctggaacatcggcgagcagaagagcatcctgtcccctctgtatgccttcgccagcgaggcagcaagggtggtgagatctatctttagccggaccctggagacagcccagaattccgtgagagtgctgcagaaggccgccatcaccatcctggatggcatctcccagtactctctgaggctgatcgatgccatgatgttcacctccgacctggccacaaacaatctggtggtcatggcctacatcaccggcggcgtggtgcagctgacctctcagtggctgacaaacatctttggcaccgtgtatgagaagctgaagccagtgctggattggctggaggagaagttcaaggagggcgtggagtttctgcgcgacggctgggagatcgtgaagttcatcagcacctgcgcatgtgagatcgtgggaggacagatcgtgacctgtgccaaggagatcaaggagtccgtgcagacattctttaagctggtgaacaagttcctggccctgtgcgccgactctatcatcatcggcggcgccaagctgaaggccctgaacctgggcgagacctttgtgacacacagcaagggcctgtacaggaagtgcgtgaagtcccgcgaggagaccggactgctgatgcccctgaaggcacctaaggagatcatcttcctggagggcgagaccctgcccacagaggtgctgacagaggaggtggtgctgaagaccggcgacctgcagccactggagcagcccaccagcgaggcagtggaggcacctctggtgggcacaccagtgtgcatcaatggcctgatgctgctggagatcaaggataccgagaagtactgtgccctggcccctaacatgatggtgacaaacaataccttcacactgaagggcggcgccccaaccaaggtgacatttggcgacgataccgtgatcgaggtgcagggctacaagtctgtgaatatcacattcgagctggatgagagaatcgacaaggtgctgaacgagaagtgcagcgcctatacagtggagctgggcaccgaggtgaacgagtttgcctgcgtggtggccgacgccgtgatcaagaccctgcagccagtgtccgagctgctgacacccctgggcatcgatctggacgagtggtctatggccacctactatctgttcgacgagagcggcgagtttaagctggcctcccacatgtactgctctttctatccccctgatgaagacgaggaggagggcgattgcgaggaggaggagtttgagcccagcacacagtacgagtatggcaccgaggacgattaccagggcaagccactggagttcggagccacctccgccgccctgcagccagaggaggagcaggaggaggattggctggacgatgactcccagcagaccgtgggccagcaggatggctctgaggacaatcagaccacaaccatccagacaatcgtggaggtgcagcctcagctggagatggagctgaccccagtggtgcagaccatcgaggtgaactctttcagcggctatctgaagctgacagataacgtgtacatcaagaacgccgacattgtggaggaggccaagaaggtgaagcctaccgtggtggtgaacgccgccaacgtgtacctgaagcacggaggaggagtggcaggcgccctgaacaaggccaccaacaatgccatgcaggtggagagcgatgactatatcgccacaaatggacccctgaaggtcggaggaagctgcgtgctgtccggacacaacctggccaagcactgtctgcacgtggtgggccctaacgtgaataagggcgaggacatccagctgctgaagtccgcctacgagaacttcaatcagcacgaggtgctgctggcccctctgctgagcgccggcatctttggcgccgatccaatccactccctgagggtgtgcgtggacaccgtgcgcacaaacgtgtacctggccgtgttcgataagaacctgtacgacaagctggtgtctagctttctggagatgaagagcgagaagcaggtggagcagaagatcgccgagatccctaaggaggaggtgaagccattcatcaccgagagcaagccttccgtggagcagaggaagcaggatgacaagaagatcaaggcctgcgtggaggaggtgacaaccacactggaggagaccaagttcctgacagagaacctgctgctgtacatcgatatcaacggcaatctgcacccagacagcgccacactggtgtccgatatcgacatcacctttctgaagaaggatgccccatatatcgtgggcgacgtggtgcaggagggcgtgctgacagccgtggtcatccccaccaagaaggccggcggcaccacagagatgctggccaaggccctgcgcaaggtgcctaccgacaattacatcaccacatatccaggccagggcctgaacggctataccgtggaggaggccaagaccgtgctgaagaagtgcaagagcgccttctacatcctgccttctatcatcagcaatgagaagcaggagatcctgggcaccgtgtcctggaacctgagggagatgctggcccacgccgaggagacacgcaagctgatgcccgtgtgcgtggagacaaaggccatcgtgagcaccatccagcggaagtataagggcatcaagatccaggagggagtggtggactacggagcaagattctacttttatacctctaagaccacagtggccagcctgatcaacacactgaatgatctgaacgagaccctggtgacaatgcccctgggctatgtgacccacggcctgaatctggaggaggccgccaggtacatgcgctccctgaaggtgccagcaaccgtgagcgtgagctctcctgacgccgtgacagcctacaacggctatctgacaagctcctctaagaccccagaggagcacttcatcgagaccatctctctggccggcagctataaggattggtcctactctggccagtccacacagctgggcatcgagtttctgaagaggggcgacaagagcgtgtactataccagcaatcccaccacattccacctggatggcgaagtgatcaccttcgacaacctgaagaccctgctgagcctgcgggaggtgagaaccatcaaggtgttcaccacagtggataacatcaatctgcacacacaggtggtggacatgtccatgacctatggccagcagtttggcccaacatacctggatggcgccgacgtgaccaagatcaagccccacaatagccacgagggcaagacattctacgtgctgcctaatgccaccaacttttccctgctgaagcaggcaggcgacgtggaggagaacccaggaccagatgacaccctgagggtggaggccttcgagtactatcacaccacagatcctagctttctgggccgctatatgtccgccctgaatcacaccaagaagtggaagtacccacaggtgaacggcctgacaagcatcaagtgggccgacaacaattgctacctggccaccgccctgctgacactgcagcagatcgagctgaagttcaacccacccgccctgcaggatgcatactatagggcaagagcaggagaggcagccaatttttgcgccctgatcctggcctattgtaacaagaccgtgggagagctgggcgatgtgcgggagacaatgagctacctgttccagcacgccaatctggactcctgcaagagagtgctgaacgtggtgtgcaagacatgtggccagcagcagaccacactgaagggcgtggaggccgtgatgtatatgggcaccctgagctacgagcagtttaagaagggcgtgcagatcccctgcacatgtggcaagcaggccaccaagtacctggtgcagcaggagtcccctttcgtgatgatgtctgcccctccagcccagtatgagctgaagcacggcacctttacatgcgcctctgagtacaccggcaattatcagtgtggccactataagcacatcaccagcaaggagacactgtactgcatcgatggcgccctgctgaccaagagctccgagtacaagggccccatcacagacgtgttctataaggagaattcttacaccacaaccatcgccaccaactttagcctgctgaagcaggccggcgatgtggaggagaaccctggaccaaagcccgtgacctataagctggacggcgtggtgtgcacagagatcgatcctaagctggacaactactacaagaaggataactcttatttcaccgagcagcccatcgacctggtgcctaatcagccttacccaaacgccagcttcgataatttcaagttcgtgtgcgacaatatcaagtttgccgatgacctgaaccagctgaccggatacaagaagccagccagccgggagctgaaggtgacattctttcctgatctgaacggcgacgtggtggccatcgactacaagcactatacaccttccttcaagaagggcgccaagctgctgcacaagccaatcgtgtggcacgtgaacaatgccaccaataaggccacatacaagccaaacacctggtgcatcagatgtctgtggtctacaaagcccgtggagaccagcaattcctttgatgtgctgaagagcgaggatgcccagggcatggacaacctggcctgcgaggacctgaagcccgtgagcgaggaggtggtggagaatcctaccatccagaaggatgtgctggagtgtaacgtgaagacaaccgaggtggtgggcgacatcatcctgaagcctgccaacaattccctgaagatcacagaggaagtgggccacaccgatctgatggccgcctacgtggacaattctagcctgaccatcaagaagccaaacgagctgagcagggtgctgggcctgaagaccctggccacacacggcctggccgcagtgaattccgtgccatgggacaccatcgccaattatgccaagcccttcctgaacaaggtggtgagcacaaccacaaacatcgtgacacggtgcctgaaccgggtgtgcaccaattacatgccatatttctttacactgctgctgcagctgtgcacctttacaaggtccaccaattctcgcatcaaggcctccatgcccaccacaatcgccaagaacacagtgaagagcgtgggcaagttctgcctggaggcctcctttaactacctgaagtcccccaatttctctaagctgatcaacatcatcatctggtttctgctgctgagcgtgtgcctgggcagcctgatctattccacagccgccctgggcgtgctgatgagcaacctgggcatgccttcctactgcaccggctatcgggagggctacctgaatagcaccaacgtgacaatcgccacctactgtacaggctctatcccatgcagcgtgtgcctgtccggcctggattctctggacacctatccttccctggagaccatccagatcacaatctcctctttcaagtgggacctgaccgcctttggcctggtggcagagtggttcctggcctatatcctgtttacaagattcttttacgtgctgggcctggccgccatcatgcagctgttctttagctacttcgccgtgcactttatctctaatagctggctgatgtggctgatcatcaacctggtgcagatggcccccatctccgccatggtgaggatgtatatcttctttgcctctttctactacgtgtggaagagctacgtgcacgtggtggacggctgcaatagctccacctgcatgatgtgctacaagaggaaccgcgccacacgcgtggagtgtaccacaatcgtgaatggcgtgcggagaagcttctacgtgtatgccaacggcggcaagggcttttgcaagctgcacaactggaattgcgtgaactgtgatacattctgtgccggcagcacctttatctccgatgaggtggcaagggacctgtccctgcagttcaagagaccaatcaatcccaccgatcagtctagctacatcgtggactccgtgacagtgaagaacggctctatccacctgtatttcgataaggccggccagaagacatacgagaggcactccctgtctcactttgtgaatctggacaacctgcgcgccaacaataccaagggcagcctgcccatcaacgtgatcgtgttcgatggcaagtccaagtgcgaggagtcctctgccaagagcgcctccgtgtactatagccagctgatgtgccagcctatcctgctgctggaccaggccctggtgtccgatgtgggcgactctgccgaggtggcagtgaagatgtttgatgcctacgtgaataccttcagcagcaccttcaacgtgccaatggagaagctgaagaccctggtggcaacagcagaggcagagctggccaagaacgtgtccctggacaatgtgctgtctaccttcatcagcgccgcccgccagggctttgtggattctgacgtggagacaaaggatgtggtggagtgcctgaagctgagccaccagtccgatatcgaggtgaccggcgacagctgtaacaattatatgctgacctacaataaggtggagaacatgacaccccgggatctgggcgcctgcatcgactgttctgccagacacatcaatgcccaggtggccaagagccacaatatcgccctgatctggaacgtgaaggacttcatgtctctgagcgagcagctgaggaagcagatccgctccgccgccaagaagaacaatctgcccttcaagctgacctgcgccaccacaaggcaggtggtgaacgtggtcaccacaaagatcgccctgaagggcggcaagatcgtgaacaattggctgaagcagctgatcaaggtgaccctggtgttcctgtttgtggccgccatcttctacctgatcacccccgtgcacgtgatgtctaagcacacagatttttctagcgagatcatcggctataaggccatcgacggaggagtgaccagggatatcgccagcaccgacacatgcttcgccaataagcacgccgatttcgacacctggtttagccagaggggcggctcctacacaaacgacaaggcctgtccactgatcgcagccgtgatcaccagggaagtgggattcgtggtgcctggactgccaggaacaatcctgaggaccacaaatggcgacttcctgcactttctgcctcgcgtgttttccgccgtgggcaacatctgctataccccatctaagctgatcgagtacaccgatttcgccacatccgcctgcgtgctggccgcagagtgtaccatctttaaggatgcctctggcaagcccgtgccttactgttatgacacaaatgtgctggagggctctgtggcctatgagagcctgcggccagataccagatacgtgctgatggacggcagcatcatccagttccccaacacatatctggagggctctgtgcgggtggtgaccacatttgacagcgagtactgccggcacggcacctgtgagagatctgaggccggcgtgtgcgtgtccacatctggcaggtgggtgctgaacaatgattactatcgcagcctgcctggcgtgttctgtggcgtggacgccgtgaatctgctgaccaacatgtttacacctctgatccagccaatcggcgccctggatatcagcgcctccatcgtggcaggaggaatcgtggcaatcgtggtgacatgcctggcctactatttcatgcggttccggagggccttcggcgagtactctcacgtggtggcctttaataccctgctgttcctgatgagcttcaccgtgctgtgcctgacccccgtgtatagcttcctgcctggcgtgtactccgtgatctacctgtatctgaccttctacctgacaaacgacgtgagctttctggcccacatccagtggatggtcatgttcacccccctggtgcctttttggatcacaatcgcctatatcatctgcatctccaccaagcacttctattggttcttttctaattacctgaagcggagagtggtgtttaacggcgtgtctttcagcacctttgaggaggccgccctgtgcacattcctgctgaacaaggagatgtacctgaagctgcggtccgacgtgctgctgccactgacccagtacaatagatatctggccctgtataacaagtacaagtatttctctggcgccatggataccacaagctacagagaggcagcatgctgtcacctggcaaaggccctgaatgatttttccaactctggcagcgacgtgctgtaccagccccctcagacctctatcacaagcgccgtgctgcagtaactagcataaccccttggggcctctaaacgggtcttgaggggttttttgtctaga

SEQ ID NO: 18 - DNA, 4012 bp, Artificial Sequence
  synthesized
gctagcgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgagcggctttcggaagatggcattcccatccggcaaggtggagggatgcatggtgcaggtgacatgtggcaccacaaccctgaatggcctgtggctggacgatgtggtgtattgccctagacacgtgatctgtaccagcgaggacatgctgaacccaaattacgaggatctgctgatcaggaagtccaaccacaatttcctggtgcaggcaggaaacgtgcagctgcgcgtgatcggccacagcatgcagaattgcgtgctgaagctgaaggtggacacagccaacccaaagacccccaagtacaagtttgtgaggatccagcctggccagacattctccgtgctggcctgctataacggctctcccagcggcgtgtaccagtgtgccatgcgccctaactttaccatcaagggctctttcctgaatggcagctgcggctccgtgggctttaacatcgactatgattgcgtgagcttctgttacatgcaccacatggagctgccaacaggagtgcacgcaggaaccgacctggagggaaacttctacggccccttcgtggacaggcagaccgcacaggcagcaggcacagatacaaccatcaccgtgaacgtgctggcctggctgtacgccgccgtgatcaacggcgaccggtggtttctgaatagattcacaaccacactgaacgatttcaatctggtggccatgaagtacaactatgagccactgacacaggaccacgtggatatcctgggaccactgagcgcccagaccggaatcgccgtgctggacatgtgcgcctccctgaaggagctgctgcagaacggcatgaatggaaggacaatcctgggaagcgccctgctggaggacgagtttaccccattcgatgtggtgagacagtgttccggcgtgacatttcaggccaccaatttctctctgctgaagcaggcaggcgatgtggaggagaaccctggaccatccgccgtgaagcgcacaatcaagggcacccaccactggctgctgctgacaatcctgacctctctgctggtgctggtgcagtctacccagtggagcctgttctttttcctgtatgagaatgcctttctgcccttcgccatgggcatcatcgccatgtccgcctttgccatgatgttcgtgaagcacaagcacgcctttctgtgcctgttcctgctgccatccctggccaccgtggcctacttcaacatggtgtatatgcctgcctcttgggtcatgaggatcatgacatggctggacatggtggatacctccctgtctggctttaagctgaaggactgcgtgatgtatgccagcgccgtggtgctgctgatcctgatgacagcaaggaccgtgtacgacgatggagcaaggagagtgtggacactgatgaatgtgctgaccctggtgtacaaggtgtactatggcaacgccctggatcaggccatctccatgtgggccctgatcatctctgtgaccagcaattattccggcgtggtgaccacagtgatgtttctggcccggggcatcgtgttcatgtgcgtggagtactgtcctatctttttcatcacaggcaacaccctgcagtgcatcatgctggtgtactgttttctgggctatttctgcacctgttactttggcctgttctgcctgctgaataggtattttcgcctgacactgggcgtgtacgactatctggtgtctacccaggagttcagatacatgaacagccagggcctgctgccccctaagaactccatcgatgccttcaagctgaatatcaagctgctgggcgtgggcggcaagccatgcatcaaggtggccacagtgcagtctaagatgagcgacgtgaagtgtaccagcgtggtgctgctgtccgtgctgcagcagctgagggtggagagctcctctaagctgtgggcccagtgcgtgcagctgcacaacgacatcctgctggccaaggataccacagaggccttcgagaagatggtgtccctgctgtctgtgctgctgagcatgcagggcgccgtggacatcaataagctgtgcgaggagatgctggataaccgcgccacactgcaggccatcgcctctgagtttagctccctgccaagctatgcagccttcgccaccgcacaggaggcatacgagcaggccgtggccaatggcgactccgaggtggtgctgaagaagctgaagaagagcctgaacgtggccaagtccgagttcgaccgggatgccgccatgcagagaaagctggagaagatggccgaccaggccatgacacagatgtataagcaggccaggtctgaggataagcgcgccaaggtgaccagcgccatgcagacaatgctgtttaccatgctgcggaagctggacaatgatgccctgaacaatatcatcaacaatgccagagacggctgcgtgcccctgaacatcatccctctgaccacagccgccaagctgatggtggtcatccctgactacaacacatataagaatacctgtgatggcaccacattcacatacgcctctgccctgtgggagatccagcaggtggtggacgccgatagcaagatcgtgcagctgagcgagatctccatggataactccccaaatctggcatggccactgatcgtgaccgccctgagggccaatagcgccgtgaagctgcagaacaatgagctgtccccagtggccctgaggcagatgtcttgcgcagcaggaaccacacagacagcctgtaccgacgataacgccctggcctactataataccacaaagggaggccggtttgtgctggccctgctgtctgacctgcaggatctgaagtgggccagattccctaagagcgacggcaccggcacaatctacaccgagctggagccaccctgccggtttgtgaccgatacacctaagggcccaaaggtgaagtacctgtatttcatcaagggcctgaacaatctgaacaggggaatggtgctgggatctctggccgcaaccgtgcgcctgcaggcaggaaacgccacagaggtgcccgccaattccaccgtgctgtctttttgtgccttcgccgtggacgcagcaaaggcatacaaggattatctggcctccggcggccagcctatcaccaattgcgtgaagatgctgtgcacccacacaggaaccggacaggccatcacagtgaccccagaggccaacatggaccaggagtcttttggcggcgccagctgctgtctgtattgccggtgtcacatcgaccaccccaatcctaagggcttctgcgatctgaagggcaagtacgtgcagatccctaccacatgtgccaatgatccagtgggctttaccctgaagaacacagtgtgcaccgtgtgcggcatgtggaagggctacggctgcagctgtgaccagctgagagagcccatgctgcagtccgccgatgcccagtcttttctgaacggcttcgccgtgtaactagcataaccccttggggcctctaaacgggtcttgaggggttttttgtctaga

SEQ ID NO: 19 - DNA, 8641 bp, Artificial Sequence
  synthesized
gctagcgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaatgtcagcagatgcacaatcatttcttaacagagtgtgcggagtgtcagcagcaagacttacaccttgcggaacaggaacatcaacagatgtagtttatagggccttcgatatctacaacgataaagtggcaggatttgcaaagttcttaaagaccaattgctgcagatttcaagagaaggacgaggatgataaccttatcgattcatactttgtggtgaagaggcatacattcagcaattaccaacacgaagaaacaatctacaaccttcttaaagattgccctgcagtggcaaagcatgacttcttcaagttcagaatcgatggagatatggtgcctcacatctcaagacaaagacttacaaagtatacgatggcagatctcgtttatgcgttgcgccatttcgacgagggtaattgtgacaccctgaaggagatcctggtcacgtataattgctgcgatgatgattactttaacaagaaggactggtatgatttcgtagagaatcctgacattcttagagtgtacgcaaaccttggagaaagagtgagacaagcactcctaaagacagttcaattctgcgacgcaatgagaaacgcaggaatcgtgggagtgcttacacttgataaccaagatcttaacggaaactggtatgactttggcgactttatacagacaacacctggatcaggagtgcctgtggtggattcatattatagcctgctgatgcctatccttacacttacaagagcacttacagcagaatcacatgtggataccgacttgaccaaaccctatattaaatgggatctgctgaaatatgactttacagaagaacgacttaaactcttcgacagatactttaaatactgggatcaaacataccaccctaactgcgtgaactgccttgatgatagatgcatccttcactgcgcaaactttaacgtgctgttctcgaccgtgtttcctcctacatcatttggacctcttgtgagaaagatctttgtggacggagtacctttcgtcgtatcaacaggataccactttagagaacttggagtagtgcataatcaagatgtgaacctacattctagccgattatcatttaaagaacttctggtttatgccgcggaccctgcaatgcacgcagcaagtggcaatttattacttgacaaacggacaacctgtttctcggttgccgcacttacaaacaatgtagctttccagaccgtaaagccagggaatttcaacaaagatttctatgacttcgccgtatcaaagggattcttcaaggagggatcatcagtggaacttaaacacttcttcttcgcccaggatggaaacgcagcaatctcagattacgattactacagatacaaccttcctacaatgtgcgatatcagacaacttctcttcgtagttgaagtggtggataaatactttgattgctacgatggaggatgcatcaacgcaaaccaagtgatcgtgaacaacttggataaatccgctggattcccgtttaataagtggggtaaagcccgcctttactacgattcaatgtcatacgaagatcaagatgcattattcgcttatacaaagaggaatgtgatccctacaatcacacaaatgaaccttaaatacgcaatctcagcaaagaatcgagcaagaacagtggcaggagtgtcaatctgctcaacaatgacaaacagacaatttcaccagaagctcctgaaatcaatcgcagcaacaagaggagcaacagtggtgatcggaacatcaaagttctatggaggttggcacaacatgctcaagaccgtgtatagcgatgttgagaatccgcatctcatgggatgggattaccctaaatgcgatagagctatgcccaatatgctgagaatcatggcatcacttgtgcttgcaagaaagcataccacatgctgctcactttcacacagattctatcgacttgcaaacgaatgcgcacaggtcctctccgagatggtgatgtgcggcgggagcttgtatgtgaaaccaggtggaacatcatcaggagatgcaacaacagcatacgcaaactcagtgtttaacatctgccaagcagtgacagctaatgtaaacgctctcttgagcactgacggaaacaagatagccgataaatacgtgcgtaatctgcagcatcgactttacgaatgcctttacagaaacagagatgtagacacggactttgtaaatgaattctatgcttaccttagaaagcatttctccatgatgatactgagtgacgatgctgttgtatgtttcaactcaacatacgcatcacaaggacttgtggcatcaatcaagaatttcaaatcagtgctttactaccagaataatgtgtttatgtcagaagcaaagtgttggacagaaactgacctcactaagggccctcacgagttctgtagccaacacacaatgcttgtgaaacaaggagatgactatgtttatctcccataccctgatccttcaagaatcttgggtgcagggtgtttcgtggatgatatcgtgaagactgacggaacacttatgatcgaaagatttgtgtcacttgcaatcgatgcataccctcttacaaagcatccgaaccaagaatacgcagatgtgtttcacctttaccttcaatacatcagaaagttgcatgatgaacttacaggacacatgcttgatatgtactcagtgatgcttacaaacgataacacatcaagatactgggaacctgaattctatgaggcaatgtacacacctcacacagtgcttcaagcagtgggagcatgcgtgctttgcaactcacaaacatcacttagatgcggagcatgcatcagaagacctttcctgtgttgcaaatgctgctacgatcacgtgatctcaacatcacacaaacttgtgctttcagtgaacccttacgtgtgcaacgcaccaggctgtgacgtaactgacgttacgcagctctatcttggaggaatgtcatactactgcaaatcacacaaacctcctatctcatttcctctttgcgcaaacggacaagtgtttggactttacaagaatacttgcgtgggatcagataacgtgacagatttcaatgctatcgcaacatgcgattggacaaacgcaggagattacatccttgcaaacacatgcacagagcgtctgaagttgtttgcggccgaaacacttaaagcaacagaagaaacatttaaactttcatacggaatcgcaacagtgagagaggtcctatcggacagggaactccacctttcatgggaagtgggcaaaccacgcccgccgcttaacagaaactacgtgtttacaggatacagagtgacaaagaattctaaggtacagatcggagaatacacatttgagaagggcgactacggagacgccgtggtgtacagagggacgactacgtataaacttaacgtgggagattactttgtgcttacatcacacacagtgatgcctctttcagcacctacacttgtgcctcaagagcattatgtccgaataacgggtctctatccgacacttaacatctcagatgaattctcgagtaacgtggcaaactaccagaaagtgggtatgcagaaatactccaccttacagggacctcctggtacaggaaagtctcatttcgcgataggtctagctctctattacccttcagcaagaatcgtgtacacagcatgctcacacgcagcagtggatgcactttgcgagaaggcgctgaaataccttcctatcgataaatgctcaagaatcatccctgcaagagcaagagtggaatgctttgataaatttaaagtgaactcaacacttgaacaatacgtgttctgtactgtaaatgctctgcctgaaactaccgcggatatcgtggtgttcgacgagatatccatggcaacaaactacgacctatcggtcgtaaacgcgcggctaagagcaaagcattatgtgtacatcggagatcctgcacaacttcctgcacctagaacattactaactaaagggacgctcgaacctgaatactttaacagtgtttgtcgcctaatgaagacgatcgggccggacatgtttcttggaacatgcagaagatgccctgcagaaatcgtggatacagtgtcagcacttgtgtacgataacaaacttaaagcacacaaagacaagtcggctcagtgtttcaagatgttttacaaaggagtgatcacacacgatgtgtcatcagcaatcaacagacctcaaatcggagtggtgagagaatttcttacaagaaaccctgcatggagaaaggcggtcttcataagtccttacaactcacagaatgccgtggcatcaaagatactcgggcttcctacacaaacagtggattcatcacaaggatcagaatacgattacgtgatctttacacaaacaacagaaacagcacactcatgcaacgtgaacagatttaacgtggcaatcacaagagcaaaggtagggatcctctgtatcatgtcagatagagatctttacgataaacttcaatttacatcacttgaaatccctagaagaaacgtggcgactctgcaggctgagaacgtgacaggattgttcaaggactgctcaaaggtaattacgggtttacatccgacacaagcacctacacacctttcagtggatacaaagttcaagactgaaggactttgcgtggatatccctggaatccctaaagatatgacatacagaagacttatctcaatgatgggatttaagatgaattaccaagtgaacggataccctaacatgtttatcacaagagaagaagcaatcagacacgtgagagcatggataggcttcgacgtcgagggatgccacgcaacaagagaagcagtgggaacaaaccttcctcttcaacttggattctccactggagtgaaccttgtggcagtgcctacaggatacgtggatacacctaacaacacagatttctcgcgagtgtcagcaaagccaccacctggagatcaatttaaacaccttatccctcttatgtacaaaggacttccttggaacgtggtgagaatcaagatagtccaaatgctatccgataccttaaagaatcttagtgaccgtgtcgtatttgtgctttgggcacacggatttgaacttacatcaatgaaatactttgtgaagatcggtcccgagcgtacatgctgcctttgcgatagaagagctacgtgtttcagtaccgcttcagatacatacgcatgctggcaccactcaataggcttcgattacgtttataatccgttcatgatagatgtgcaacaatggggattcacgggcaatctgcagagcaaccacgatctttactgccaagtgcacggaaacgcacacgtggcatcatgcgatgcaatcatgacaagatgccttgcagtgcacgaatgctttgtgaagcgggtcgattggacaatcgaataccctatcatcggagatgaacttaagataaatgcagcatgcagaaaggtccagcacatggtggtgaaagcagcacttcttgcagataaatttcctgtgcttcacgatatcggaaaccctaaagcaatcaaatgcgtgcctcaagcagatgtggaatggaaattctatgacgcacaaccttgctcagataaagcatacaagatagaggaactattctatagttacgcaacacactcagataaatttacagatggagtgtgcctgttctggaattgcaacgtggatagataccctgcaaactcaatcgtgtgcagatttgatacaagagtgctttcaaaccttaaccttccaggttgtgacggcggcagtctatatgttaataagcacgcatttcacacacctgcattcgataagtccgcattcgtcaatttaaagcagctacctttcttctattattcagattcaccttgcgaatcacacggaaagcaggttgtcagtgacatcgattacgtgcctcttaaatcagcaacatgtattaccaggtgtaatcttggaggagccgtctgtcgacatcatgcaaacgaatacagactttaccttgatgcatacaacatgatgatctccgccgggttctccctatgggtgtacaaacaatttgatacatacaacctttggaacacatttacaagacttcaatcacttgagaacgttgcgttcaatgtagtcaataagggacacttcgacggtcaacagggtgaggttcctgtgtcaatcatcaacaataccgtttatactaaagttgacggcgtggatgtggaactcttcgagaataagactacgcttcctgtgaatgttgccttcgagttgtgggcaaagcgcaatatcaaacctgtgcctgaagtgaagatactcaataaccttggagtggatatcgcagcaaacacagtgatctgggattacaagagggacgcacctgcacacatctcaacaatcggagtgtgctcaatgacagatatcgcaaagaagccgactgaaacaatctgcgcacctcttactgtattcttcgacggaagagtggatggacaagtggatttattccgaaatgcaagaaacggagtgcttatcacagaaggatcagtgaaaggacttcaaccttcagtgggacctaaacaagcatcacttaacggagtgactctgataggcgaggccgtgaagactcagtttaactactacaagaaagtagacggtgtcgtccagcagctgcccgagacctatttcacacaatcacggaatctgcaggagttcaaacctagatcacaaatggaaatcgatttcctggagcttgcaatggatgaatttatcgaaagatacaaacttgaaggatacgcatttgaacacatcgtgtacggagatttcagtcattcacaacttggaggacttcaccttcttattggcctagccaaacgtttcaaagaatcacctttcgagctcgaagatttcattccaatggattcaacagtgaagaattatttcattactgacgcccagacgggatcatcaaagtgtgtatgctcagtgatcgatctactactagacgatttcgttgaaattattaaatcacaagacttgagtgtagttagtaaggttgtgaaggtcacaatcgattacacagaaatctcatttatgctttggtgcaaagatggacacgtggaaacattctatcccaaacttcaatcatcacaagcatggcaacctggagtggccatgccgaatttgtataagatgcagagaatgcttcttgagaagtgtgaccttcagaattatggagattcagcaacacttcctaaaggaatcatgatgaacgtggcaaagtatactcaactttgccaataccttaacacacttacacttgcagtgccttacaacatgagagtgatccacttcggtgcagggtcggacaaaggagtggcacctggtactgctgtccttagacaatggcttcctacaggaacacttcttgtggattcagatcttaacgatttcgtctccgatgcagattcaaccctcattggtgactgtgcaacagtgcacacagcaaacaagtgggacttaataatatcagatatgtacgatcctaagactaagaatgtaacgaaagagaatgactcaaaggaaggtttcttcacctatatctgcggatttatccaacagaagttagctcttggaggatcagtggcaatcaagattacggaacactcatggaacgcagatctttacaaacttatgggacactttgcatggtggaccgcgttcgttacaaacgtaaacgcgtcgtcctcagaagcatttcttatcggatgcaactaccttgggaaaccaagagagcagatcgatggatacgtgatgcacgcaaactacatcttctggaggaacacaaaccctatccaactttcatcatactcactcttcgacatgtcaaagttcccgcttaaacttagagggactgccgtaatgtcgcttaaagaaggacaaatcaacgatatgatactcagcctcctaagtaaagggaggcttatcatcagagagaataatagagtggtgatctcatcagatgtgcttgtgaacaactaactagcataaccccttggggcctctaaacgggtcttgaggggttttttgtctaga

SEQ ID NO: 20 - DNA, 50 bp, Artificial Sequence
  synthesized
agtgatgttcttgttaacaactaaacgaacaatgtttgtttttcttgttt

SEQ ID NO: 21 - DNA, 50 bp, Artificial Sequence
  synthesized
agtcaaattacattacacataaacgaacttatggatttgtttatgagaat

SEQ ID NO: 22 - DNA, 66 bp, Artificial Sequence
  synthesized
tgatcttctggtctaaacgaactaaatattatattagtttttctgtttggaactttaattttagcc

SEQ ID NO: 23 - DNA, 50 bp, Artificial Sequence
  synthesized
gcaaccaatggagattgattaaacgaacatgaaaattattcttttcttgg

SEQ ID NO: 24 - DNA, 134 bp, Artificial Sequence
  synthesized
ttgaactttcattaattgacttctatttgtgctttttagcctttctgctattccttgttttaattatgcttattatcttttggttctcacttgaactgcaagatcataatgaaacttgtcacgcctaaacgaac

SEQ ID NO: 25 - DNA, 50 bp, Artificial Sequence
  synthesized
tttagatttcatctaaacgaacaaactaaaatgtctgataatggacccca

SEQ ID NO: 26 - DNA, 265 bp, Artificial Sequence
  synthesized
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaag

SEQ ID NO: 27 - DNA, 294 bp, Artificial Sequence
  synthesized
tgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaata

SEQ ID NO: 28 - DNA, 463 bp, Artificial Sequence
  synthesized
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 29 - DNA, 12 bp, Artificial Sequence
  synthesized
taataataataa

SEQ ID NO: 30 - DNA, 4364 bp, Artificial Sequence
  synthesized
gctagcattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaaggtggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagcctgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataataataagatatctgatcttctggtctaaacgaactaaatattatattagtttttctgtttggaactttaattttagccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaaggatccgtgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaactagcataaccccttggggcctctaaacgggtcttgaggggttttttgtctaga